(19) United States
(12) Patent Application Publication
(10) Pub. No.: US 2023/0189731 A1
(43) Pub. Date: Jun. 22, 2023

(54) METHOD FOR IDENTIFICATION, DISTINCTION AND SELECTION OF PLANTS OF THE GLYCINE GENUS, RESISTANT OR SUSCEPTIBLE TO TARGET SPOT CAUSED BY THE FUNGUS CORYNESPORA CASSIICOLA , METHOD FOR INTROGRESSION INTO PLANTS OF THE GLYCINE GENUS OF ALLELES OF RESISTANCE TO TARGET SPOT CAUSED BY THE FUNGUS CORYNESPORA CASSIICOLA, NUCLEIC ACID MOLECULE AND ITS USE, DETECTION KIT, METHOD FOR GENOTYPING TARGET SPOT-RESISTANT GLYCINE TARGET PLANTS AND TARGET SPOT-RESISTANT GLYCINE PLANTS

(71) Applicant: TMG TROPICAL MELHORAMENTO E GENÉTICA S.A., CAMBE (BR)

(72) Inventors: Alexandre GARCIA, Londrina (BR); Anderson Rotter MEDA, Londrina (BR); João Vitor Maldonado DOS SANTOS, Londrina (BR); Marcella Viana DE SOUSA, Londrina (BR); Luan Gonçalves CRUZ, Londrina (BR)

(73) Assignee: TMG TROPICAL MELHORAMENTO E GENÉTICA S.A., CAMBE (BR)

(21) Appl. No.: 17/924,935

(22) PCT Filed: Sep. 2, 2020
(86) PCT No.: PCT/BR2020/050353

§ 371 Date: Nov. 11, 2022

(30) Foreign Application Priority Data
May 12, 2020 (BR) BR 102020009417 3

(51) Int. Cl. A01H 1/04; C12Q 1/6895; A01H 1/00; A01H 6/54

(52) U.S. Cl. CPC A01H 1/045; C12Q 1/6895; A01H 1/1255; A01H 6/542; C12Q 2600/13; C12Q 2600/156; C12Q 1/6858

(57) ABSTRACT

The present invention relates to a method for identifying and selecting plants resistant to a fungal disease comprising the steps of; (a) extraction of nucleic acid from a plant; (b) analysis of extracted nucleic acid for the presence of markers associated with increased fungal resistance within a single chromosome interval; and (c) selection of the plants that have these markers.

Furthermore, the invention also relates to a method for introgression into plants of fungal disease resistance alleles comprising the steps of; (a) crossing parents of plants identified by the first embodiment method with other parents that do not have this resistance; (b) select progenies possessing markers associated with increased resistance to fungal disease using the method as defined in the first achievement; and (c) backcross in one or more cycles the selected progenies with the recurrent genitor to develop new progenies.

### FIELD OF INVENTION

The present invention relates to the field of plant biology and biotechnology. Specifically, the present invention relates to a method of plant breeding in order to identify plants by means of molecular markers, with higher resistance to diseases, more specifically plants of the genus Glycine and fungal diseases.

### BACKGROUND OF THE INVENTION

Soybean belongs to the botanical genus Glycine, more precisely to the family Fabaceae (legumes). Some 727 genera and 19,325 species are recognized (LEWIS, G. P.; SCHRIRE, B. D.; MACKINDER, B. A.; LOCK, J. M. Legumes of the World. Royal Botanic Gardens, Kew. p. 577, 2005) representing one of the largest families of Angiosperms and also one of the leading ones from an economic point of view.

This family has a cosmopolitan distribution and its main characteristic, although there are exceptions, is the vegetable-type fruit (pod). In addition, it ranges from tree species to annual herbaceous species, many of great economic importance, primarily, to feed (soy, beans, among others).

In addition, representatives of this family still have great ecological importance, as they are well adapted to the first colonization and exploitation of diverse environments, mainly due to their associations with nitrogen-fixing bacteria or with ectomycorrhizae. Bacteria of the genus Rhizobium, located in root nodules found in many species, convert atmospheric nitrogen into ammonia, a soluble form that can be used by other plants, resulting in species extremely valuable as suppliers of natural fertilizers (LEWIS, G. P. Legumes of Bahia. Royal Botanic Gardens, Kew. p. 369, 1987).

Soy (Glycine max) is one of the most important representatives of the Fabaceae family. In the 1970s, soy became consolidated as the main crop in Brazilian agribusiness. The producer has used all means to increase the use of technology, in order to reduce their costs, increase their productivity, and thereby improve their profitability. Thus, soybean productivity jumped from 2,823 kg/ha in the 2006/07 harvest, to 3,394 kg/ha in the 2017/18 harvest, a 20% increase (Monitoring Brazilian grain harvest, v. 6-2018/19 Crop-Tenth survey, Brasilia). The most recent data show that soy generates revenues of R$148.6 billion in 2018 and the highest revenue in exports, having reached US$40 billion in the same year (Cleonice de Carvalho, et al. Brazilian soybean yearbook 2019. Santa Cruz do Sul: Editora Gazeta Santa Cruz, P. 14, 2019).

The worldwide demand for quality animal protein, especially poultry, is continuously increasing around the world (HENCHION, M.; McCARTHY, M.; RESCONI, V. C.; TROY, D. Meat consumption: trends and quality matter. Meat Science, v.98, p.561-568, 2014.). Thus, this growing demand also generates an increase in the demand for protein meals used in the manufacture of animal feed, usually derived from soybeans (Embrapa (2011) Soybean Production Technologies, Central Region of Brazil 2012 and 2013. Londrina PR. Embrapa Soja).

World consumption of soybeans in crop year 2019/20 is projected to increase to 352 million tons, up from 345 million tons consumed in 2018/19 (Cleonice de Carvalho, et al. Brazilian soybean yearbook 2019. Santa Cruz do Sul: Editora Gazeta Santa Cruz, P. 14, 2019).

Furthermore, the area under soybean cultivation grew when comparing the period 2017/18 with 2018/19 from 124.52 million hectares to 125.64 million hectares (USDA, Global Market Analysis, February 2020).

Due to the economic importance of soybean in the Brazilian agricultural scenario, soybean breeding programs aim to develop cultivars that are more productive and resistant to diseases and pests present in the different regions of Brazil. A key part of the success of breeding programs for the selection of resistant genotypes lies in the use of inoculum sources (fungal isolates) representative of local diversity with known virulence spectrum and aggressiveness (Bermejo, Gabriela Rastelli. Genetic diversity of Brazilian isolates of Phakopsora pachyrhizi (Sydow & Sydow)/Gabriela Rastelli Bermejo; orientation Mayra Costa da Cruz Gallo de Carvalho-Bandeirantes: State University of Northern Parana, 2016).

In this scenario, improving soybean for resistance or tolerance to various pathogens is crucial to decrease constraining factors and maximize productivity. Among the pathogens, the fungus Corynespora cassiicola stands out (Berk. & M. A. Curtis) C. T. Wei, the etiological agent of the disease known as target spot. It is considered one of the most economically important diseases for soybean production in Brazil, especially in the Cerrado region (Almeida AMR, Ferreira L P, Yorinori J T, Silva J F V, Henning A A, Godoy C V, Costamilan L M, Meyer M C (2005) Soybean diseases. In: Kimati H, Amorim L, Rezende J A M, Bergamin Filho A, Camargo L E A (Eds.). Handbook of Plant Pathology—Vol. 2. Diseases of Cultivated Plants. 4. ed. Sao Paulo SP. Editora Agronômica Ceres. pp. 570-588).

The aforementioned fungus is found in virtually all soybean-growing regions of Brazil. Believed to be native and with the ability to infect a large number of plant species, such as cotton, increasing its adaptability in areas where soybean-cotton crop succession is performed (GALBIERI, R.; ARAÚJO, D. C. E. B.; KOBAYASTI, L.; GIROTTO, L.; MATOS, J. N.; MARANGONI, M. S.; ALMEIDA, W. P.; MEHTA, Y. R. Corynespora leaf blight of cotton in Brazil and its management. American Journal of Plant Sciences 5: 3805-3811. 2014).

This microorganism can survive on crop remains and infected seeds, which is one form of dissemination. It is estimated that the disease can cause a yield reduction of 24%, with variations between 8-42% in soybean crops with high disease pressure (GALBIERI, R.; ARAÚJO, D. C. E. B.; KOBAYASTI, L.; GIROTTO, L.; MATOS, J. N.; MARANGONI, M. S.; ALMEIDA, W. P.; MEHTA, Y. R. Corynespora leaf blight of cotton in Brazil and its management. American Journal of Plant Sciences 5: 3805-3811. 2014)

Severe but sporadic outbreaks have been observed in the cooler regions of the South and in the high Cerrados regions. Susceptible cultivars can suffer complete premature defoliation, pod rot, and stalk spotting. Through infection in the pod, the fungus can reach the seed and thus be spread to other areas. Infection, in the suture region of the developing pods, can result in necrosis, pod splitting, and germination or rotting of the still-green kernels (Embrapa (2011) Soybean Production Technologies, Central Region of Brazil 2012 and 2013. Londrina PR. Embrapa Soja.).

Conditions of high relative humidity and mild temperatures are favorable for leaf infection. The most common symptoms are leaf spots, with a yellowish halo and dark punctuation in the center, which cause severe defoliation. Stains also occur on the stem and pod. The fungus can infect roots, causing root rot and intense sporulation (Henning et al., 2005, supra).

In this sense, in general, infection by this pathogen can be observed in all parts of the plants above ground (GALBIERI, R.; ARAÚJO, D. C. E. B.; KOBAYASTI, L.; GIROTTO, L.; MATOS, J. N.; MARANGONI, M. S.; ALMEIDA, W. P.; MEHTA, Y. R. Corynespora leaf blight of cotton in Brazil and its management. American Journal of Plant Sciences, v.5, p. 3805-3811, 2014; 2. HARTMAN, G. L.; RUPE, J. C.; SIKORA, E. J.; DOMIER, L. L.; DAVIS, J. A.; STEFFEY, K. L. Compendium of soybean diseases and pests. In: HARTMAN et al. (Ed.). 5th. ed. The American Phytopathological Society, St. Louis, Mo. Paul, Minn. 201p., 2015).

The progress of target spot in the field is slower compared to Asian rust, but once the disease is established, it is difficult to control. The recommended management strategies for this disease are: rotation with non-host crops, seed treatment, chemical control at correct doses and intervals, and use of resistant cultivars. However, the lack of information on the reaction of soybean cultivars to this disease makes its management difficult, and chemical control is used as one of the most viable alternatives (MEYER, M.; GODOY, C.; VENANCIO, W.; TERAIVIOTO, A. Balanced management. Cultivar Magazine, v.165, p.03-0′7, 2013). In the case of chemical control, the association of multisite fungicides should always be recommended and the management should always begin in a preventive manner. The use of fungicide alone and in a curative manner can eliminate more sensitive populations of the fungus, increasing the frequency of the less sensitive (Teramoto, A.; Meyer, M. C.; Suassuna, N. D.; Cunha, M. G. In vitro sensitivity of Corynespora cassiicola isolated from soybean to fungicides and field chemical control of target spot. Summa Phytopathologica, v.43, n.4, p.281-289, 2017).

The genetic architecture for disease resistance has been established by several associative mapping studies, which point to a monogenic or polygenic character, depending on the type of interaction between pathogen and host. The same studies allowed the identification of DNA polymorphisms at the major effector loci associated with resistance responses. In this context, associative mapping studies are of great use for plant breeding programs by making it possible to map loci and gain knowledge about the position of a gene and its adjacent region. Furthermore, these studies allow the interpretation of possible resistance mechanisms and the prediction of the inheritance of the trait in controlled crosses, in addition to contributing to synteny or comparative mapping analysis and gene cloning (Xuehui Huang and Bin Han, Natural Variations and Genome-Wide Association Studies in Crop Plants, Annual Review of Plant Biology, 65: 531-551, 2014)

Linear mixed models have been developed and applied in associative mapping to reduce the number of false-positive associations caused by population structure and relationship (YU, J. M.; PRESSOIR, G.; BRIGGS, W. H.; VROH BI, I.; YAMASAKI, M.; DOEBLEY, J. F.; MCMULLEN, M. D.; GAUT, B. S.; NIELSEN, D. M.; HOLLAND, J. B.; KRESOVICH, S.; BUCKLER, E. S. A unified mixed-model method for association mapping that accounts for multiple levels of relatedness. Nature Genetics, v.38, p.203-208, 2006; ZHANG, Z.; ERSOZ, E.; LAI, C.-Q.; TODHUNTER, R. J.; TIWARI, H. K.; GORE, M. A.; BRADBURY, P. J.; YU, J.; ARNETT, D. K.; ORDOVAS, J. M.; BUCKLER, E. S. Mixed linear model approach adapted for genome-wide association studies. Nature Genetics, v.42, p.355-360, 2010.).

Molecular markers have been used in identifying polymorphisms associated with disease resistance. In breeding programs, the marker-assisted selection approach (SAM) has been widely used because it allows the identification of disease resistance or other characteristics already in the early stages and early stages of plant development.

Using SAM, unfavorable alleles can be eliminated or greatly reduced in the first few generations, which allows for the evaluation and selection of an optimal number of plants in the field. In another application, SAM can facilitate the introgression of favorable alleles from resistance sources into elite strains (Shi, Z., Liu, S., Noe, J. et al. SNP identification and marker assay development for high-throughput selection of soybean cyst nematode resistance. BMC Genomics 16, 314 (2015). https://doi.org/10.1186/s12864-015-1531-3).

Resistant cultivars are usually developed by transferring resistance alleles from germplasm, often unadapted, to elite cultivars. Due to the wide genetic variability of fungal species and their constant adaptations, the emergence of new isolates that challenge the genetic resistance already introduced in elite cultivars is common. Thus, it is essential to explore a broad genetic base in germplasm to ensure the longevity of resistance (ALZATE-MARIN, A L.; CERVIGNI, G. D. L.; MOREIRA, M. A; (2005) Marker assisted selection in the development of disease resistant plants, with emphasis on common bean and soybean. Brazilian Phytopathology. v.30, no.4, p.333-342).

In this context, broad genome association studies are of great use for plant breeding programs because they allow the mapping of loci that control qualitative or quantitative traits (QTLs—Quantitative Trait Loci), and for providing knowledge about the position of a gene and its adjacent region. Furthermore, such studies allow the interpretation of evolutionary mechanisms and the prediction of progeny from controlled crossings, as well as contributing to the analysis of synteny or genetic mapping and gene cloning.

A genetic map is a graphical representation of a genome (or a part of a genome, such as a single chromosome) where the distances between reference points on the chromosome are measured by the recombination frequencies between these points. A genetic reference point can be any one of a variety of known polymorphic markers, for example, but not limited to molecular markers, such as SSR-type markers (Simple Sequence Repeats) RFLP-type markers (Restriction Fragment Length Polymorphism) or SNP-type markers (Single nucleotide polymorphism). Also, sSR-type markers can be derived from genomic or expressed nucleic acids (for example, ESTs (Expressed sequence tags)).

Gene-associated markers or QTLs, once mapped and evaluated for influence on phenotypic variation, can be used for SAM, which makes the process of choosing a particular genotype fast and efficient, making it a tool of great contribution to plant breeding (Collins, P J, et al, Marker assisted breeding for disease resistance in Crop Plants. Biotechnologies of Crop Improvement, v3, 41-47, 2018).

Recently, marker-assisted selection has increased the efficiency of traditional soybean breeding programs. Furthermore, the availability of integrated linkage maps of the soybean genome containing increasing densities of public soybean markers has facilitated soybean genetic mapping and SAM applications (Cregan et al. (1999) “An Integrated Genetic Linkage Map of the Soybean Genome” Crop Sci. 39:1464-1490).

SNPs (Single nucleotide polymorphism) are markers that consist of a differentiated shared sequence based on a single nucleotide.

SNPs between homologous DNA fragments and small insertions and deletions (indels), known collectively as single nucleotide polymorphisms (SNPs) have been shown to be the most abundant source of DNA polymorphisms in humans (Kwok P.-Y., Deng Q., Zakeri H., Nickerson D. A., 1996 Increasing the information content of STS-based genome maps: identifying polymorphisms in mapped STSs. Genomics 31: 123-126; Y. L. Zhu, Q. J. Song, D. L. Hyten, C. P. Van Tassell, L. K. Matukumalli, D. R. Grimm, S. M. Hyatt, E. W. Fickus, N. D. Young and P. B. Cregan Genetics Mar. 1, 2003 vol. 163 no. 3 1123-1134).

SNPs are suitable for developing high-throughput and easy-to-automate genotyping methods because most SNPs are biallelic, thus simplifying genotyping approaches and analyses. (Lin C H, Yeakley J M, McDaniel T K, Shen R (2009) Medium- to high-throughput SNP genotyping using VeraCode microbeads. Methods Mol Biol 496: 129-142; Yoon M S, Song Q J, Choi I Y, Specht J E, Hyten D L, et al. (2007) BARCSoySNP23: a panel of 23 selected SNPs for soybean cultivar identification. Theor Appl Genet 114: 885-899). Based on SNP analysis and bioinformatics tools, linkage disequilibrium and haplotype analysis can be quantified. Furthermore, another point to be considered is that the use of molecular markers for assisted improvement, including SNPs, detects genetic information without interference from the environment, in transcribed and non-transcribed regions, bringing the advantage of the possibility of eliminating or reducing the need for time-consuming and laborious phytopathological analyses. The breeder can identify individuals carrying markers linked to the allele of interest, as disease resistance, resulting in time and resource savings (ALZATE-MARIN, A L.; CERVIGNI, G. D. L.; MOREIRA, M. A; (2005) Marker assisted selection in the development of disease resistant plants, with emphasis on common bean and soybean. Brazilian Phytopathology. v.30, no.4, p.333-342).

Currently, the main form of control of target spot is through the use of fungicides. However, fungicides from the carboxamide chemical group have been reducing their control efficiency probably due to the presence of resistant isolates of Corynespora cassiicola to methyl-benzimidazole-carbamate fungicides (MBC) (GODOY, C. V.; UTIAMADA, C. M.; MEYER, M. C.; CAMPOS, H. D.; PIMENTA, C. B.; JACCOUD-FILHO, D. S. Efficiency of fungicides for the control of target spot, Corynespora cassiicola, in the 2013/14 crop: summarized results of cooperative trials. Londrina: Embrapa Soja, 2014. 6p. (Embrapa Soja. Technical Circular 104).

Thus, there is a need to use complementary methods for effective disease management, such as genetic resistance in cultivars. Despite the economic importance of soybeans and the threat of target spot, so far, there are no scientific publications describing sources (genotypes) for disease resistance, much less studies of genetic inheritance, description of resistance genes/locus and neither studies on the location of possible resistance genes to Corynespora cassiicola.

The present invention identifies soybean genome SNPs associated with soybean resistance to the fungus Corynespora cassiicola and discloses a method for identifying and selecting plants resistant to this pathogen. In addition, it also reveals a method for introgression into plants of resistance alleles to the fungus Corynespora cassiicola in soybean.

The advantages of the invention will be evident in the description of the invention provided herein.

### SUMMARY OF THE INVENTION

In one aspect, the invention relates to a method for identifying, distinguishing and selecting plants of the genus Glycine, resistant or susceptible, to target spot caused by the fungus Corynespora cassiicola which comprises:
(a) Extraction of nucleic acid from a plant of the genus Glycine;
(b) Analysis of extracted nucleic acid for the presence of one or more alleles of the molecular markers associated with increased resistance or susceptibility to Corynespora cassiicola within a range of 37.69-37.85 Mpb of chromosome 17;
(c) Selection of the plants that possess the mentioned alleles of the markers.

In one embodiment of the method, one or more markers are located in the genomic region of the genes or in the ranges of the genes Glyma.17g224300 (SEQ ID NO: 1), Glyma.17g223800 (SEQ ID NO: 2), Glyma.17g223900 (SEQ ID NO: 3), Glyma.17g224000 (SEQ ID NO: 4), Glyma.17g224100 (SEQ ID NO: 5), Glyma.17g224200 (SEQ ID NO: 6), Glyma.17g224500 (SEQ ID NO: 8), Glyma.17g224600 (SEQ ID NO: 9), Glyma.17g224700 (SEQ ID NO: 10), Glyma.17g224800 (SEQ ID NO: 11), Glyma.17g224900 (SEQ ID NO: 12), Glyma.17g225000 (SEQ ID NO: 13), Glyma.17g225100 (SEQ ID NO: 14), Glyma.17g225200 (SEQ ID NO: 15), Glyma.17g225300 (SEQ ID NO: 16), Glyma.17g225400 (SEQ ID NO: 17), Glyma.17g225500 (SEQ ID NO: 18). In a preferred embodiment, markers are located in the genomic region of genes or in the ranges of genes selected from the group consisting of Glyma.17G224300 (SEQ ID NO: 1), Glyma.17G224400 (SEQ ID NO: 7) and Glyma.17G224500 (SEQ ID NO: 8) and even more preferentially, said marker is a SNP selected from the group consisting of ss715627273, ss715627288, ss715627282, ss715627290, ss715627293, ss715627289, ss715627296, ss715627297, ss715627265, ss715627264, ss715627310, ss715627276, ss715627274, ss715627280 and ss715627279, or combinations thereof, or any other molecular marker in a range up to 5 cM or 1 Mbp from said group, even more preferably said marker is a SNP selected from the group consisting of ss715627288, ss715627273 and ss715627282, or combinations thereof, or any other molecular marker within 5 cM or 1 Mbp of that group.

In one form of embodiment, the method comprises identifying the markers by any amplification methodologies, or by use of probes, or by any type of sequencing (e.g. tGBS or directed sequencing).

In another form of embodiment, the method the plant of the genus Glycine is Glycine max.

In another aspect, the invention relates to a method of introgressing into plants of the genus Glycine alleles of resistance to target spot caused by the fungus Corynespora cassiicola, comprising:

(a) Crossing parents of plants of the genus Glycine identified by the method as defined in any of claims 1 to 6 with other parents lacking said resistance;

(b) Select progenies possessing markers associated with increased resistance or reduced susceptibility to Corynespora cassiicola by the method as defined in claim 1; e

(c) Backcross in one or more cycles the selected progenies with the recurrent genitor to develop new progenies.

In a further aspect, the invention relates to a nucleic acid molecule capable of hybridizing with any of the SEQ ID NO: 19, 20, 21, 22, 23, 24, 25, 26, 27, 28, 29, 30, 31, 32, or 33, or subsequences thereof having at least 15 consecutive nucleotides, or sequences with at least 90% sequence identity. [0042] In a further aspect, the invention also relates to the use of a nucleic acid molecule as defined above in the methods of the invention.

In a further aspect, included in the invention is a detection kit comprising at least two nucleic acid molecules as defined above.

In a further aspect, the invention relates to a method for genotyping target Glycine plants resistant to target spot, comprising analyzing the presence in the DNA of the target plant for one or more markers associated with target spot resistance, selected from the group consisting of ss715627273, ss715627288, ss715627282, ss715627290, ss715627293, ss715627289, ss715627296, ss715627297, ss715627265, ss715627264, ss715627310, ss715627276, ss715627274, ss715627280 and ss715627279, or combinations thereof.

In a further aspect, the invention relates to a target spot resistant Glycine plant obtained by an introgression method as defined above.

### BRIEF DESCRIPTION OF THE FIGURES

FIG. 1 refers to the diagrammatic scale developed by Soares et al (2009) and adjustments to a 1-9 rating scale for assessing Corynespora cassiicola severity in soybean and cotton leaf tissue, with respective genotype responses.

FIG. 2 refers to associative mapping of SNPs associated with resistance to Corynespora cassiicola.

FIG. 3 refers to the block plot in high linkage disequilibrium under the region where the most significant SNPs were mapped.

FIG. 4 refers to the genes identified in the range corresponding to the block in linkage disequilibrium in which the most significant SNPs are found.

FIG. 5 refers to the allelic substitution effect for SNPs detected by three markers in the reaction (severity) to Corynespora cassiicola in a test progeny from a cross between a resistant and susceptible parent.

### DETAILED DESCRIPTION OF THE INVENTION

Unless defined differently, all technical and scientific terms used herein have the same meaning as understood by a person skilled in the subject matter to which the invention pertains. The terminology used in describing the invention is intended to describe particular embodiments only, and does not intend to limit the scope of the teachings. Unless otherwise stated, all numbers expressing quantities, percentages and proportions, and other numerical values used in the descriptive report and claims, should be understood as being modified in all cases by the term “about”. Thus, unless otherwise stated, the numerical parameters shown in the descriptive report and in the claims are approximations that may vary, depending on the properties to be obtained.

The practice of the present invention will employ, unless otherwise indicated, conventional methods of chemistry, biochemistry, and recombinant DNA techniques, within the skill of the art. Such techniques are explained fully in the literature. Take a look, e.g. Fundamental Virology, 2nd Edition, vols. I & II (B. N. Fields and D. M. Knipe, eds.); T. E. Creighton, Proteins: Structures and Molecular Properties (W.H. Freeman and Company, 1993); A. L. Lehninger, Biochemistry (Worth Publishers, Inc., current edition); Sambrook, et al, Molecular Cloning: A Laboratory Manual (2nd Edition, 1989) Methods In Enzymology (S. Colowick and N. Kaplan eds., Academic Press, Inc.).

The following terms are defined, and may be used within the scope of the present invention in order to facilitate general understanding.

Gene: the basic physical and functional unit of heredity, being composed of DNA and capable of being transcribed into RNA. Some genes act as instructions for polypeptides;

QTL: quantitative Trait Loci, which refers to a quantitative trait locus. It is a locus that correlates with the variation of a quantitative trait in the phenotype of a population of organisms;

Locus: refers to a position or location that a particular gene or any other genetic element or factor contributing to a trait occupies in a chromosome of a given species.

Allele: variant forms of a given gene, which occupy the same region on homologous chromosomes, affecting the same trait, but in a different way. The same gene can have several alleles;

Chromosome: is an organized package of DNA found in the nucleus of the cell that can contain several genes;

Genotype: refer to the alleles, or variant forms of a gene, that are understood by an organism;

Genetic map: It is a graphical representation of a genome or a part of a genome, such as a single chromosome. It is a description of the genetic linkage relationships between loci on one or more chromosomes in a given species. For each genetic map, the distances between loci are measured by the recombination frequencies between them. Recombination between loci can be detected using a variety of markers;

Linkage disequilibrium: is defined in the context of the invention as the relative frequency of gamete types in a population of many individuals in a single generation. If the frequency of an allele A is p, a is p′, B is q and b is q′, then the expected frequency (without linkage disequilibrium) of genotype AB is pq, Ab is pq′, aB is p′q and ab is p′q′. Any deviation from the expected frequency is called a linkage disequilibrium Two loci are said to be “genetically linked” when they are in linkage disequilibrium.

Genetic linkage: refers to a trait association in inheritance due to the location of genes in close proximity on the same chromosome, measured by the percentage of recombination between loci (centi-Morgan, cM). The distances between loci are usually measured by the recombination frequency between loci on the same chromosome. The further apart two loci are from each other, the more likely it is that recombination will occur between them. Conversely, if two loci are close together, a recombination is less likely to happen between them. As a rule, 1 centi-Morgan is equal to 1% recombination between loci. When a QTL can be indicated by multiple markers, the genetic distance between markers at the ends (flankers) is indicative of the size of the QTL. For purposes of this invention, “genetically linked to a marker” can be considered that the marker is not more than 10 cM apart, preferably 5 cM, more preferably 2 cM and even more preferably 1 cM of the genetic determinant that confers resistance.

Molecular markers: are DNA fragments that are associated with a specific region of the genome, which can be monitored. They refer, in other words, to indicators that are used in methods to visualize differences in nucleic acid sequences. Marker molecules can take the form of short DNA sequences, as a sequence involving a single nucleotide polymorphism, where a single base pair change occurs. They can also take the form of longer DNA sequences, such as microsatellites, with 10 to 60 base pairs.

Germplasm: refers to the totality of genotypes in a population. It can also refer to plant material, for example a group of plants that are repositories of several alleles.

Resistance: refers to the ability of a plant to restrict the growth and development of a specific pathogen and/or the resulting signal/symptom, when compared to susceptible plants under similar environmental conditions and pathogen pressure. Includes both partial resistance and full resistance to infection (for example, infection by a pathogen that causes target spotting). A resistant plant will show no or few symptoms of the disease. A susceptible plant can either be a non-resistant plant or have lower levels of resistance to infection compared to a resistant plant.

Introgression: refers to natural or artificial processes in which genomic regions of one species, variety or cultivar are transferred to the genome of another species, variety or cultivar by crossing over. The process can optionally be completed by backcrossing between an individual and its recurrent parent.

Crossover: refers to the fusion of gametes via pollination to produce an offspring, including both self-fecundation (when pollen and ovule are from the same plant) or cross-fertilization (when pollen and egg are from different plants).

Marker assisted selection (SAM): is a process by which phenotypes are selected on the basis of molecular genotypes. Marker assisted selection includes the use of molecular markers to identify plants or populations that possess the genotype of interest in breeding programs.

PCR (polymerase chain reaction): refers to a method of producing relatively large quantities of specific regions of DNA, allowing various analyses based on these regions.

PCR Initiators (“primers”): relatively small fragments of single-stranded DNA used in the PCR amplification of specific regions of DNA.

Probe: refers to molecules or atoms that are able to recognize and bind to a specific target molecule, allowing detection of the target molecule. In particular, for purposes of this invention, “probe” refers to a sequence of labeled DNA or RNA that can be used to detect and/or quantify a complementary sequence by molecular hybridization.

The following detailed description refers to genetic markers and related methods for identification of such markers, genotyping of plants of the genus Glycine, and methods for marker-assisted breeding of these plants.

### Nucleic Acid Molecules-Loci, Primers and Probes

The loci pertaining to the present invention comprise bounded genomic sequences comprising one or more molecular markers, including a polymorphism identified in Table 5, Table 7 or Table 8, as shown in the SEQ ID NOS: 19 a 33, or is adjacent to one or more of these polymorphisms.

In one aspect of the invention, isolated nucleic acid sequences are provided (oligonucleotides) that are capable of hybridizing to the polymorphic loci of the present invention. In certain embodiments, for example, that come from initiators, such molecules comprise at least 15 nucleotide bases. Molecules useful as primers can hybridize under high-stringency conditions to one or more strands of a DNA segment at a polymorphic locus of the invention. Primers for DNA amplification are provided in pairs, i.e., forward primers (or F)” or “reverse (or R)”. One primer will be complementary to one DNA strand at the locus and the other primer will be complementary to the other DNA strand at the locus, i.e. preferentially, sequences that are at least 90% included, more preferably 95%, or 100% identical to a sequence as described in SEQ ID Nos: 19 to 48, or to sub-sequences of at least 15 nucleotides. Furthermore, it is understood that such primers can hybridize to a sequence at the locus that is distant from the polymorphism, for example, at least 5, 10, 20, 50, 100, 200, 500 or even about 1,000,000 nucleotides away from the polymorphism. The design of an initiator of the invention will depend on factors well known in the art, for example, avoiding a repetitive sequence.

In addition to this, it should be remembered here that, although preferred functions may be mentioned in relation to some oligonucleotides, it is obvious that a given oligonucleotide may assume several functions, and may be used in different forms in accordance with the present invention. As the person skilled in the art knows, in some situations, a primer can be used as a probe and vice versa, as well as being applicable in hybridization procedures, detection etc. Thus, it is noted that products according to the present invention, especially, inter alia, oligonucleotides, are not limited to the uses shown here, but rather, the uses should be interpreted broadly, independent of the use indicated here. Furthermore, when an oligonucleotide is described as being useful as a probe that can bind to an amplicon, the subject matter expert also understands that the complementary sequence of this oligonucleotide is equally useful as a probe to bind to the same amplicon. The same is true for the sequences described as useful as primers. Additionally, It is also obvious that any initiator suitable for a multiplex protocol can also, within the meaning and scope of the present invention, be used in a singleplex protocol. The same applies to a suitable primer for a real-time PCR protocol, that can be used in a conventional PCR protocol, within the meaning of the present invention.

The person skilled in the art, in this regard, understands that the oligonucleotides of the present invention, i.e., the primers and probes, need not be completely complementary to a part of the target sequence. The primer can exhibit sufficient complementarity to hybridize with the target sequence and perform the intrinsic functions of a primer. The same applies to a probe, that is, a probe can exhibit sufficient complementarity to hybridize with the target sequence and perform the intrinsic functions of a probe. Therefore, a primer or a probe in one embodiment need not be completely complementary to the target sequence. In one embodiment, the primer or probe can hybridize or ring with a part of the target to form a double strand. The conditions for hybridization of a nucleic acid are described by Joseph Sambrook et al. Molecular Cloning, A Laboratory Manual, Cold Spring Harbor Laboratory Press, Cold Spring Harbor, N.Y. (2001) and Haymes et al. Nucleic Acid Hybridization, A Practical Approach, IRL Press, Washington, D.C. (1985).

In another aspect of the invention, is the kit comprising at least two primers as described above.

Another aspect of the nucleic acid molecules of the invention are the hybridization probes. In one embodiment, such probes are oligonucleotides comprising at least 15 nucleotide bases and a detectable marker. The purpose of such molecules is to hybridize, for example, under high-stringency conditions, to a DNA strand in a segment of nucleotide bases that includes or is adjacent to a polymorphism of interest. Such oligonucleotides are preferentially at least 90%, more preferentially 95% identical to the sequence of a segment of Glycine DNA at a polymorphic locus, or to a fragment of it comprising at least 15 nucleotide bases. But specifically, the polymorphic locus is selected from the group consisting of SEQ ID NO: 19-33.

The detectable marker can be a radioactive element or a dye. In preferred aspects, the hybridization probe still comprises a fluorescent marker and a quencher, for example, for use in hybridization assays such as Taqman® assays, available from AB Biosystems. In this case, the detectable marker and the quencher are located at opposite ends. For SNP detection assays, it is useful to provide such markers and quenchers in pairs, for example, where each molecule for detection of a polymorphism has a distinct fluorescent marker and quencher, different for each polymorphism.

More specifically, with respect to the TaqMan™ probe, an oligonucleotide, whose 5′ terminal region is modified with a fluorophore and the 3′ terminal region is modified with a quencher, is added to the PCR reaction. It is also understood that it is possible to bind the fluorophore in the 3′ terminal region and the quencher in the 5′ terminal region. The reaction products are detected by fluorescence generated after the 5′ exonuclease activity->3′ of DNA polymerase. The fluorophores, which refer to fluorescent compounds that emit light with the excitation by light having a shorter wavelength than the light that is emitted, can be, but are not limited to, FAM, TAMRA, VIC, JOE, TET, HEX, ROX, RED610, RED670, NED, Cy3, Cy5, and Texas Red. The quenchers can be, but are not limited to, 6-TAMRA, BHQ-1,2,3 and MGB-NFQ. The choice of the fluorophore-quencher pair can be made so that the excitation spectrum of the quencher has an overlap with the emission spectrum of the fluorophore. One example is the FAM-TAMRA pair, FAM-MGB, VIC-MGB and so on. An expert on the subject will know how to recognize other appropriate pairs.

It is not necessary that there be complete complementarity between the sequences, as long as the differences do not completely impair the ability of the molecules to form a double-stranded structure. Therefore, for a nucleic acid molecule to be able to serve as a primer or probe, it must be sufficiently complementary in sequence to allow the formation of a double-stranded structure under the hybridization conditions used.

In a preferred embodiment, a nucleic acid molecule will hybridize to a segment of Glycine DNA shown in SEQ ID NO: 1 to 33.

### Polymorphism Detection

SNPs are the result of a variation in sequence and new polymorphisms can be detected by sequencing genomic DNA or cDNA molecules.

In one aspect, polymorphisms in a genome can be determined by comparing the cDNA sequence of different strains. Although the detection of polymorphisms by cDNA sequence comparison is relatively convenient, the evaluation of the cDNA sequence does not allow information about the position of the introns in the corresponding genomic DNA. In addition, polymorphisms in the non-coding sequence cannot be identified from the cDNA. This can be a disadvantage, for example when using cDNA-derived polymorphisms as markers for genomic DNA genotyping. More efficient genotyping assays can be designed if the scope of polymorphisms includes those present in the single non-coding sequence.

Genomic DNA sequencing is more useful than cDNA for identifying and detecting polymorphisms. Polymorphisms in a genome can be determined by comparing the genomic DNA sequence of different strains. However, the genomic DNA of higher eukaryotes usually contains a large fraction of repetitive sequence and transposons. Genomic DNA can be sequenced more efficiently if the coding/unique fraction is enriched by subtracting or eliminating repetitive sequences.

There are several well-known strategies in the technique that can be employed to enrich the sample in coding sequences/unique sequences. Examples of these include the use of enzymes that are sensitive to cytosine methylation, the use of the McrBC endonuclease to cleave the repetitive sequence and the printing of microarrays of genomic libraries that are then hybridized with repetitive sequence probes.

A method for reducing repetitive DNA comprises constructing reduced representation libraries by separating the repetitive sequence of genomic DNA fragments from at least two varieties of a species, fractioning the separated genomic DNA fragments based on nucleotide sequence size, and comparing the sequence of fragments in a fraction to determine polymorphisms. More particularly, these methods for identifying polymorphisms in genomic DNA comprise digesting the total genomic DNA of at least two variants of a eukaryotic species with a methylation-sensitive endonuclease to provide a pool of digested DNA fragments. The average nucleotide length of the fragments is shorter for DNA regions characterized by a lower percentage of 5-methylated cytosine. Such fragments are separable, e.g. by gel electrophoresis, on the basis of nucleotide length. A fraction of DNA with shorter than average nucleotide length is separated from the digested DNA pool. DNA sequences in a fraction are compared to identify polymorphisms. Compared to the coding sequence, The repetitive sequence is most likely to comprise 5-methylated cytosine, e.g. in the -CG- and -CNG-sequence segments. In one mode of the method, genomic DNA from at least two different inbred varieties of a Glycine is digested with a methylation-sensitive endonuclease selected from the group consisting of enzymes such as Aci I, Apa I, Age I, Bsr FI, BssHII, Eag I, Eae I, Hha I, HinP II, Hpa II, Msp I, MspMII, Nar I, Not I, Pst I, Pvu I, Sac II, Sma I, Stu I and Xho I to provide a physically separated pool of digested DNA, for example by gel electrophoresis. Fractions of comparable size of DNA are obtained from the digested DNA of each of the aforementioned enzymes. DNA molecules from the comparable fractions are inserted into vectors or isolated to construct reduced representation libraries of genomic DNA clones that are sequenced and compared to identify polymorphisms.

Another method for enrichment of coding sequences/single sequence consists of constructing reduced representation libraries (using methylation-sensitive enzymes or not) by printing microarrays of the library on a nylon membrane, followed by hybridization with probes made from repetitive elements known to be present in the library. The repetitive sequence elements are identified and the library is reorganized by choosing only the negative clones. Such methods provide reduced representation genomic DNA segments of a plant that has genomic DNA comprising DNA regions with relatively higher levels of methylated cytosine and DNA regions with relatively lower levels of methylated cytosine.

In addition, microarrays can be used (DNA chip) of soy available in the technique, such as SoySNP50K (Song Q, Hyten DL, Jia G, Quigley CV, Fickus EW, Nelson RL, et al. (2013) Development and Evaluation of SoySNP50K, a High-Density Genotyping Array for Soybean. PLoS ONE 8(1): e54985). This panel has been widely exploited for soybean genetic studies, allowing the identification of associations between SNPs and disease resistance, among other traits.

### Determination of Polymorphisms in DNA Samples of Glycine

Polymorphisms in DNA sequences can be detected by a variety of methods well known in the art. DNA samples include, but are not limited to, the genotypes shown in Table 1.

For example, methods to detect SNPs and Indels include single base extension methods (SBE). Examples of SBE methods include, but are not limited to, those disclosed in U.S. Pat. Nos. 6,004,744; 6,013,431; 5,595,890; 5,762,876; and 5,945,283. SBE methods are based on extending a nucleotide primer that is immediately adjacent to a polymorphism to incorporate a detectable nucleotide residue after primer extension. In certain embodiments, the SBE method uses three synthetic oligonucleotides. Two of the oligonucleotides serve as PCR primers and are complementary to the sequence of the soybean genomic DNA site that flanks a region containing the polymorphism to be tested. After amplification of the soybean genome region containing the polymorphism, the PCR product is mixed with the third oligonucleotide (called the extension initiator), which is designed to hybridize to the amplified DNA immediately adjacent to the polymorphism in the presence of DNA polymerase and two differentially labeled dideoxynucleoside triphosphates. If polymorphism is present in the template, one of the labeled didesoxynucleosidetriphosphates can be added to the primer at a single base chain length. The allele present is then inferred by determining which of the two differential markers was added to the extension primer. Homozygous samples will result in the incorporation of only one of the two marked bases e, therefore, only one of the two markers will be detected. Heterozygous samples have both alleles present and therefore direct the incorporation of both markers (on different molecules of the extension primer) and, therefore, both markers will be detected.

In a preferred method for detecting polymorphisms, SNPs and Indels can be detected by methods disclosed in U.S. Pat. Nos. 5,210,015; 5,876,930; and 6,030,787 in which an oligonucleotide probe is used with a fluorescent dye at 5′ and a quencher at 3′ from the probe. When the probe is intact, the proximity of the fluorescent dye to the quencher results in suppression of the fluorescence of the fluorescent dye, e.g. by Forster-type energy transfer. During PCR, the forward and reverse primers hybridize to a specific sequence of the target DNA that flanks a polymorphism while the hybridization probe hybridizes to the polymorphism-containing sequence in the amplified PCR product. In the subsequent PCR cycle, DNA polymerase with 5→3′ exonuclease activity breaks the probe and separates the fluorescent dye from the quencher, resulting in increased fluorescence of the fluorescent dye.

A useful test is available from AB Biosystems as the Taqman® test, which employs four synthetic oligonucleotides in a single reaction that simultaneously amplifies soybean genomic DNA, discriminates the alleles present, and directly provides a signal for discrimination and detection. Two of the four oligonucleotides serve as PCR primers and generate a PCR product that encompasses the polymorphism to be detected. Two others are allele-specific fluorescence resonance energy transfer probes (FRET). In the trial, two FRET probes with different fluorescent reporter dyes are used, where a single dye is incorporated into an oligonucleotide that can ring with high specificity with only one of the two alleles. Useful reporter dyes include, among others, 6-carboxy-4,7,2 ‘,7’-tetrachlorofluorecein (TET)2′-chloro-7′-phenyl-1,4-dichloro-6-carboxyfluorescein (VIC) and 6-carboxyfluorescein phosphoramidite (FAM). A useful inhibitor is 6-carboxy-N, N, N′, N′-tetramethyl-rhodamine (TAMRA). Also, the 3′ end of each FRET probe is chemically blocked so that it cannot act as a PCR primer. A third fluorophore used as a passive reference is also present, for example rhodamine X (ROX) to help with subsequent normalization of the relevant fluorescence values (correcting volumetric errors in the reaction set-up). The amplification of the genomic DNA is started. During each PCR cycle, FRET probes bind in an allele-specific manner to the templates of DNA molecules. The ringed FRET probes (but not the non-ringed ones) are degraded by TAQ DNA polymerase as the enzyme meets the 5′ end of the ringed probe, thereby releasing the fluorophore from the vicinity of its quencher. After PCR, the fluorescence of each of the two fluorescents, as well as the passive reference, is determined fluorometrically. The normalized fluorescence intensity for each of the two dyes will be proportional to the amounts of each allele initially present in the sample e, therefore, the genotype of the sample can be inferred.

PCR primers are designed (a) to have a size of about 15 to 25 bases and sequences that hybridize at the polymorphic locus, (b) has a melting temperature in the range 57° C. to 60° C., corresponding to a ringing temperature of 52° C. to 55° C., (c) produces a product that includes the polymorphic site and typically has a size ranging from 75 to 250 base pairs. However, there are PCR techniques that allow amplification of larger fragments of 1000 or more base pairs. Primers are preferably located at the locus so that the polymorphic site is at least 1 base away from the 3′ end of each primer. However, it is understood that PCR primers can be up to 1000 base pairs or more away from the polymorphism and still provide amplification of a corresponding DNA fragment containing the polymorphism that can be used in soybean genotyping assays.

Directed sequencing techniques can be applied for polymorphism detection. The development of increasingly inexpensive and rapid sequencing technologies has led to the facilitation of large-scale detection of polymorphisms in various model and non-model plant species (Kumar S, Banks TW, Cloutier S. SNP Discovery through Next-Generation Sequencing and Its Applications. International journal of plant genomics vol. 2012 (2012): 831460). The development and improvement of freely available, open-source bioinformatics software has accelerated the discovery of SNPs. It is worth noting that the facilitation of whole genome sequencing has led to the discovery of several million SNPs in different organisms.

### Using Polymorphisms to Establish Marker Associations and Resistance to Target Spot

Polymorphisms at the loci of this invention can be used to identify associations of markers and target-spot resistance that are inferred from statistical analysis of genotypic and phenotypic data from members of a population

Various types of statistical analyses can be used to infer the association of markers and resistance to target spot from phenotype/genotype data, but a basic idea is to detect molecular markers, i.e., polymorphisms, for which alternative genotypes have significantly different average phenotypes. For example, if a given marker locus “A” has three alternative genotypes (AA, Aa and aa) and if these three classes of individuals have significantly different phenotypes, then we will infer that locus “A” is associated with the desired characteristic. The significance of differences in phenotype can be tested by various types of standard statistical tests, such as linear regression of genotypes of molecular markers in the phenotype or analysis of variance (ANOVA). The statistical software packages available on the market, commonly used to do this type of analysis include linear mixed models (MLM) developed by the MVP packages (YIN et al., 2018) GAPIT (TANG et al., 2016) and FarmCPU (LIU et al., 2016) with the Emma matrix algorithms (MVP) and VanRaden (GAPIT and FarmCPU). When many molecular markers are tested simultaneously, an adjustment, such as the Bonferroni correction, is made to the level of significance necessary to declare an association.

Often, the goal of an association study is not simply to detect associations of markers and desired traits, but to estimate the locations of genes that affect the trait directly in relation to the locations of the markers. In a simple approach to this goal, a comparison is made between marker locations of the magnitude of the difference between alternative genotypes or the level of significance of this difference. It is inferred that the trait genes are located closer to the marker(s) that have the largest associated genotypic difference. The genetic linkage of additional marker molecules can be established by a genetic mapping model, as, without limitation, the flanking marker model reported by Lander et al. (Lander et al. 1989 Genetics, 121: 185-199) and interval mapping, based on maximum likelihood methods, and implemented in the software package MAPMAKER/QTL (Lincoln and Lander, mapping Genes Controlling Quantitative Traits Using MAPMAKER/QTL, Whitehead Institute for Biomedical Research, Massachusetts, (1990).) Additional software includes Qgene, Version 2.23 (1996) Department of Plant Breeding and Biometrics, 266 Emerson Hall, Cornell University, Ithaca, N.Y.).

A maximum likelihood estimate is calculated (MV) for the presence of a marker, together with a MV that assumes no QTL effect, to avoid false positives. A log 10 of an odds ratio (“odds ratio” or LOD) is then calculated as: LOD=log 10 (MV for the presence of a QTL/MV without QTL bound). The LOD score essentially indicates how much more likely the data is to arise assuming the presence of a QTL versus in its absence. The LOD limit value to avoid a false positive with a given confidence, for example 95%, depends on the number of markers and the length of the genome.

For the development of the present invention, a set of genotypes was used (as per table 1) which were inoculated with isolates of Corynespora cassiicola that showed virulence considered high and intermediate (table 2). These genotypes were evaluated for resistance to target spot, resistant genotypes were selected as described in Table 4. Included within the scope and for the purposes of the present invention are all genotypes considered resistant and highly resistant, which can be used in breeding programs as sources of resistance to target spot. More preferentially are the genotypes considered highly resistant, selected from the group consisting of PI 71506, PI 153230, PI 567310B, PI 587802, PI 587860, PI 407999-1 and PI 548984.

### Construction of Genetic Maps

In another aspect of the invention, the polymorphism at the sites of the invention is mapped on the soybean genome as a physical map of the soybean genome comprising positions on the map of two or more polymorphisms, as indicated in Tables 5, 7 and 8.

More specifically, the present invention describes the identification of genetic markers (SNPs or combinations of two or more SNPs) that can be used to identify alleles associated with resistance or tolerance to target spot in plants. More specifically, markers are present in a 110-kpb interval on chromosome 17 of G. max, associated with target spot resistance.

### Marker-Assisted Improvement and Marker Assisted Selection

When a locus has been located in close proximity to molecular markers, these markers can be used to select improved aspects of the trait without the need for phenotypic analysis in each selection cycle. In marker assisted breeding and marker assisted selection, the associations between loci and markers are initially established through mapping analysis. In the same process, it is determined which alleles of the molecular markers are linked to favorable alleles of the locus/loci being studied. Subsequently, alleles of the markers associated with favorable locus/loci alleles are selected in the population. This procedure will improve the “value” of the trait to be selected, in this case resistance to the target spot, provided there is a sufficiently close link between markers and the locus involved in resistance. The degree of linkage required depends on the number of generations of selection because, in each generation, there is an opportunity to break the association by recombination.

There are a few ways to quantify the level of efficiency of molecular markers for selecting genotypes of interest. One of the main ways is in the use of accuracy calculations and type I and II error rates. Accuracy is a measure that shows how effective a marker is in detecting resistant and susceptible individuals. This calculation is used as a way to accurately indicate how close a genotypic result is to the phenotypic data for the trait under study. High accuracy values indicate high efficiency in the selection of individuals using molecular markers. Type I and II error rates, on the other hand, are measures that quantify possible flaws in the correlation of phenotypic and genotypic data. Type I errors, also called false-positive, are results in which the genotypic data indicate the presence of a resistance allele, while the phenotypic data suggest that the samples analyzed are susceptible to the trait. In contrast, type II, or false-negative errors, demonstrate the genotypic presence of susceptible alleles in samples with disease resistance phenotypes. Low Type I and II error values decrease the probability of eliminating resistant and susceptible materials, respectively, by using molecular markers (Maldonado dos Santos, J. V., Ferreira, E. G. C., Passianotto, A. L. d. L. et al (2019). Association mapping of a locus that confers southern stem canker resistance in soybean and SNP marker development. BMC Genomics 20, 798; Bruna Bley Brumer. Morphological, molecular and pathogenic characterization of Diaporthe aspalathi isolates and validation of SNPs markers associated with stem canker resistance in soybean. Master's Dissertation. Universidade Estadual de Londrina-UEL-PR-2016; Adriano Consoni Camolese. Phytophthora root rot in soybean: Identification of a recessive resistance gene and validation of SNPs for use in molecular marker assisted selection. Master's Dissertation. State University of Londrina-UEL-PR-2015).

Associations between specific marker alleles and favorable alleles can also be used to predict which types of progeny may segregate from a given cross. This prediction can allow the selection of appropriate parents for generation populations from which new combinations of favorable alleles are assembled to produce a new pure lineage. For example, if strain A has marker alleles previously associated with favorable alleles at locations 1, 20, and 31, while strain B has marker alleles associated with favorable effects at locations 15, 27, and 29, a new strain can be developed by crossing A×B and selecting progenies that have favorable alleles at all 6 loci.

Molecular markers are used to accelerate the introgression of genes or chromosomal segments into new genetic backgrounds (that is, in a diverse range of germplasm). Simple introgression involves crossing a donor line of a new trait to an elite line and, then select and backcross F1 plants repeatedly to the elite parent (recurrent) while selecting the maintenance of the gene of interest/chromosome segment. Over several generations of backcrossing, the genetic background of the original line is gradually replaced by the genetic background of the elite through recombination and segregation. This process can be accelerated by selecting the alleles of the recurrent parent through molecular markers. This approach is known as marker-assisted backcrossing.

Finally, it is possible to establish a “fingerprint” or fingerprint of a lineage, as the combination of alleles in a set of two or more marker loci. High density fingerprints can be used to establish and trace the identity of germplasm, which has utility in establishing a database of trait-marker associations to benefit a soybean breeding program, as well as protecting the intellectual property of the germplasm.

Thus, according to a first aspect of the invention, the present invention provides methods for identifying and selecting plants resistant to a fungal disease comprising the steps of:
(a) Extraction of nucleic acid from a plant;
(b) Analysis of extracted nucleic acid for the presence of one or more markers associated with increased fungal resistance within a chromosome interval;
(c) Selection of the plants that have these markers.

Preferably, the method is directed toward identification of plants of the genus Glycine, more specifically plants of the species Glycine max.

Preferentially, resistance to the fungus is resistance to Corynespora cassiicola, the etiologic agent of target spot.

Obtaining a nucleic acid sample from a plant can be accomplished by standard DNA isolation methods well known in the art, as described supra.

Analysis for the presence of markers can be done by PCR, probes, or sequencing. In one form of embodiment, the nucleic acid molecules (PCR primers and probes) comprise sequences from SEQ ID Nos: 19-48, or sub-sequences of these that are at least 15 nucleotides in length. Also included in the scope of the invention are sequences that are at least 90% identical to SEQ ID Nos: 19-48 or their sub-sequences.

With respect to fungal disease, the method of the present invention preferably relates to the fungus Corynespora cassiicola, which causes the disease called Target Spot, and resistance or tolerance to said disease is conferred by a locus or QTL.

Preferably, the marker is a SNP-type marker (Single nucleotide polymorphism).

A marker corresponds to an amplification product generated by the amplification of a nucleic acid from Glycine sp., for example by polymerase chain reaction (PCR) using two primers. In this context, “molecular marker” refers to an indicator that is used in methods to visualize differences in characteristics of nucleic acid sequences (polymorphisms). A molecular marker “linked to” or “associated with” a gene capable of providing resistance to target spot can therefore refer to SNPs.

Furthermore, the markers can also be detected by using probes or targeted sequencing (tGBS).

Detection of a molecular marker may, in some embodiments, comprise the use of one or more primer sets that can be used to produce one or more amplification products. In a first embodiment, such primer sets can hybridize to a part of the nucleotide sequences as shown in SEQ ID Nos: 19 a 33 (Table 10) or sub-sequences of these that are at least 15 nucleotides in length. Still, they are included in the scope of the invention, sequences that are at least 90% identical to SEQ ID Nos: 19-48 or its subsequences.

In another embodiment of the present invention, the markers are located in the genes or ranges of the Glyma.17g224300 genes (SEQ ID NO: 1), Glyma.17g223800 (SEQ ID NO: 2), Glyma.17g223900 (SEQ ID NO: 3), Glyma.17g224000 (SEQ ID NO: 4), Glyma.17g224100 (SEQ ID NO: 5), Glyma.17g224200 (SEQ ID NO: 6), Glyma.17g224400 (SEQ ID NO: 7), Glyma.17g224500 (SEQ ID NO: 8), Glyma.17g224600 (SEQ ID NO: 9), Glyma.17g224700 (SEQ ID NO: 10), Glyma.17g224800 (SEQ ID NO: 11), Glyma.17g224900 (SEQ ID NO: 12), Glyma.17g225000 (SEQ ID NO: 13), Glyma.17g225100 (SEQ ID NO: 14), Glyma.17g225200 (SEQ ID NO: 15), Glyma.17g225300 (SEQ ID NO: 16), Glyma.17g225400 (SEQ ID NO: 17), Glyma.17g225500 (SEQ ID NO: 18) present on chromosome 17 of Glycine max.

In a third embodiment of the present invention, markers are preferably located in the adjacent regions of the selected genes of the group consisting of Glyma.17g224300 (SEQ ID NO: 1), Glyma.17g224400 (SEQ ID NO: 7) and Glyma.17g224500 (SEQ ID NO: 8) present on chromosome 17 of Glycine max.

In a fourth embodiment of the present invention, the markers are SNPs selected from the group consisting of ss715627273, ss715627288, ss715627282, ss715627290, ss715627293, ss715627289, ss715627296, ss715627297, ss715627265, ss715627264, ss715627310, ss715627276, ss715627274, ss715627280 and ss715627279, or combinations thereof.

In a fifth embodiment of the present invention, the SNPs are preferably ss715627288, ss715627273 and ss715627282.

In a sixth embodiment of the present invention, the plant is preferably of the species Glycine max.

In a further aspect, the present invention relates to a method of introgressing into plants of the genus Glycine alleles of resistance to target spot caused by the fungus Corynespora cassiicola, comprising the steps of:
(a) Crossing parents of plants of the genus Glycine identified by the method as defined in the previous embodiments with other parents lacking this resistance;
(b) Select progenies possessing markers associated with increased resistance to Corynespora cassiicola using the method as defined in the previous achievements; e
(c) Backcross in one or more cycles the selected progenies with the recurrent genitor to develop new progenies.

In a further aspect, the present invention relates to a method for genotyping target Glycine plants resistant to target spot, comprising analyzing the presence in the DNA of the target plant for one or more markers associated with resistance to target spot, selected from the group consisting of ss715627273, ss715627288, ss715627282, ss715627290, ss715627293, ss715627289, ss715627296, ss715627297, ss715627265, ss715627264, ss715627310, ss715627276, ss715627274, ss715627280 and ss715627279, or combinations thereof. In a further aspect, the invention comprises commercial or customized kits comprising such nucleic acid molecules.

In a further aspect, the invention comprises a method for genotyping target Glycine plants resistant to target spot, comprising analyzing the presence in the DNA of the target plant for one or more markers associated with target spot resistance, selected from the group consisting of ss715627273, ss715627288, ss715627282, ss715627290, ss715627293, ss715627289, ss715627296, ss715627297, ss715627265, ss715627264, ss715627310, ss715627276, ss715627274, ss715627280 and ss715627279, or combinations thereof.

Preferably, the present invention relates to methods for producing a commercial variety resistant to Corynespora cassiicola from susceptible varieties, comprising performing the above introgression method using conventional breeding techniques. The present invention is further described by the examples below, which are intended only to exemplify one of the innumerable ways of carrying out the invention, however, without limiting its scope.

### EXAMPLES

### Example 1

### Soybean Genotypes Evaluated

A total of 520 soybean genotypes were evaluated in this study. These are Glycine max accessions from various centers of origin, with most originating from Asia (62.5%) and America (23.4%). The list of samples used in this study can be seen in Table 1.

TABLE 1

| PI Id | Material | Source | Maturation Group |
|---|---|---|---|
| PI 71506 | No. 94 | China | IV |
| PI 153230 | B-34 | Germany | ZZ |
| PI 567310 B | (Hei huang dou) | China | V |
| PI 587802 | Da li huang | China | VII |
| PI 587860 | Qi yue bai | China | V |
| PI 407999-1 | KAERI 544-5 | South Korea | V |
| PI 548984 | Tracy-M | United States | VI |
| PI 347550 A | Primorskaia 494 | Russia | I |
| PI 417115 | Kyushu 16 | Japan | VII |
| PI 87606 | Oiarukon | North Korea | IV |
| PI 319537 A | Tono No. 1 | China | Z |
| PI 603572 | Chun bai dou | China | V |
| PI 594762 | Tian yang qing dou | China | X |
| PI 534646 | Flyer | United States | IV |
| PI 576857 | LYON | United States | VI |
| PI 640911 | AxN-1-55 | United States | II |
| PI 424079 | 74079 | South Korea | IV |
| PI 424611 A | KAS 681-24 | South Korea | IV |
| PI 424611 B | KAS 681-24 | South Korea | IV |
| PI 424612 | KAS 681-25 | South Korea | IV |
| PI 84578 | S-1 | South Korea | III |
| PI 290116 A | Hodoninska Zluta | Hungary | Z |
| PI 248398 | Illinois 301 | United States | II |
| PI 360954 | Fiskeby IV | Sweden | ZZZZ |
| PI 258384 | A | Poland | Z |
| PI 153274 | U487 | Belgium | I |
| PI 209335 | No. 5 | Japan | IV |
| PI 89002 | 5947 | China | III |
| PI 407659 B | (Dun haj hun mao czi) | China | III |
| PI 189872 | Commercial Huilerie Nord | France | Z |
| PI 495017 C | (Beijing da qing don) | China | IV |
| PI 196148 | Akasaya-1 | Japan | III |
| PI 90763 | 7570 | China | IV |
| PI 238925 | Roudnicka Black | Czech Republic | Z |
| PI 360964 | Smena | Russia | ZZ |
| PI 417095 | Kuro sakigake | Japan | Z |
| PI 507352 | Toiku 152 | Japan | II |
| PI 200480 | Itate No. 14 | Japan | III |
| PI 297551 | Viola Manchu Mediasch | Hungary | I |
| PI 88788 | 5913 | China | III |
| PI 194624 | 291-1-2 | Sweden | ZZ |
| PI 361085 B | (L.117) | Romania | Z |
| PI 567597 C | (Xiao huang dou) | China | III |
| PI 547842 | L77-1863 | United States | III |
| PI 547876 | L85-3059 | United States | III |
| PI 595843 | Flint | United States | II |
| PI 548318 | Dunfield | China | III |
| PI 548524 | Weber | United States | I |
| PI 548663 | Dowling | United States | VIII |
| PI 543855 | Newton | United States | II |
| PI 546039 | OT89-01 | Canada | ZZ |
| PI 618613 | MN0902CN | United States | Z |
| PI 398447 | KAS 210-3 | South Korea | V |
| PI 506624 | Chouhin Hitashi 13 | Japan | VI |
| PI 548659 | Braxton | United States | VII |
| PI 416806 | Aso Aogari (Kyushu 27) | Japan | VIII |
| PI 408042 | KAERI 574-1 | South Korea | V |
| PI 170889 | 4/38S5 | South Africa | VI |
| PI 594754 | Ji wo dou | China | IX |
| PI 594527 | Chang ting wu chang qing dou | China | IX |
| PI 567132 C | MARIF 2799 | Indonesia | IX |
| PI 307882 C | No. 47 | India | X |
| PI 587687 E | (Xiao li dou No. 1) | China | VII |
| PI 424442 | KAS 544-24 | South Korea | VI |
| PI 229320 | Ginjiro | Japan | VI |
| PI 416937 | Houjaku Kuwazu | Japan | VI |
| PI 593999 A | — | South Korea | V |
| PI 407987 | KAERI 542-6 | South Korea | V |
| PI 567126 | MARIF 2793 | Indonesia | IX |
| PI 417011 | Kari Mame | Japan | VI |
| PI 567104 B | MARIF 2769 | Indonesia | IX |
| PI 567076 | MARIF 2674 | Indonesia | VII |
| PI 567397 | Lu huang dou | China | V |
| PI 230979 | No. 12 | Japan | VI |
| PI 559371 | Hood 75 | United States | VI |
| PI 587883 B | (Jiu yue lao shu dou) | China | VII |
| PI 587668 B | (Hui mei dou) | China | VI |
| PI 518722 | Nan nong 493-1 | China | VII |
| PI 587886 | Bai dou | China | VI |
| PI 476918 | Trung Quoc Xanh a | China | VI |
| PI 632648 | Cao bang 1 x U8354 | Vietnam | VI |
| PI 506764 | Hyuuga | Japan | VII |
| PI 561373 | Fen dou 34 | China | V |
| CD 201 | CD 201 | Brazil | VI |
| NA 5909 RG | NA 5909 RG | Brazil | VI |
| Tapir 82 | Tapir 82 | Brazil | VII |
| PI 518671 | Williams 82 | Brazil | III |
| PI 632667 | H 9 | Vietnam | IV |
| PI 543832 | Buckshot 723 | United States | VII |
| PI 594675 | Huang dou No. 1 | China | V |
| PI 68494 | 78 | China | III |
| PI 68621 | 116 | China | III |
| PI 84580 | S-3 | South Korea | II |
| PI 84957 | Yamki daizu | Japan | III |
| PI 85626 | Y-425 | South Korea | IV |
| PI 86102 | Konshurei No. 234 | Japan | II |
| PI 86972-1 | Pakute | South Korea | II |
| PI 87531 | 4274 | China | I |
| PI 87617 | Miyongaikon | North Korea | III |
| PI 88508 | Showa No. 1-4 | China | II |
| PI 153311 | C.N.S. 24 (De Charlien) | France | I |
| PI 153313 | Kleverhof | Germany | I |
| PI 157431 | Ic-san | South Korea | IV |
| PI 358313 | Kitami | Japan | II |
| PI 398735 | KAS 331-1 | South Korea | IV |
| PI 408132 | KAS 640-1 | South Korea | IV |
| PI 417524 | Zolta Swhn | Poland | ZZ |
| PI 424298 | KAS 300-10 | South Korea | IV |
| PI 499957 | — | China | III |
| PI 507354 | Tokei 421 | Japan | I |
| PI 507686 C | (Kisinjevskaja 19) | Moldova | I |
| PI 567651 | Shang cai er cao ping ding shi | China | IV |
| PI 594599 | Chang de chun hei dou | China | IV |
| PI 632661B | (H 3) | Vietnam | IV |
| PI 174862 | No. 10207 | India | VI |
| PI 269518 C | (Koolat) | Pakistan | VI |
| PI 323564 | H 67-15 | India | VIII |
| PI 374162 | M-9 | India | VIII |
| PI 378693 A | — | Japan | VIII |
| PI 408046 | KAERI 575-4 | South Korea | V |
| PI 423913 | Mizukuguri | Japan | VIII |
| PI 423966 | Kumaji 2 | Japan | VIII |
| PI 458122 | KAS 301-16 | South Korea | VI |
| PI 476905 A | Nguu mao hong | China | V |
| PI 567079 | MARIF 2677 | Indonesia | VIII |
| PI 567082 A | MARIF 2680 | Indonesia | VIII |
| PI 567346 | Niu mao huang dou | China | V |
| PI 587905 | Xiao huang dou | China | VII |
| PI 587996 B | (Ji wo dou) | China | VII |
| PI 594669 | Liu yue mang | China | V |
| PI 605779 C | Sample 42 | Vietnam | VIII |
| PI 605779 D | Sample 42 | Vietnam | VII |
| PI 615487 | Xanh tien dai | Vietnam | V |
| PI 628803 | BR-7 | Brazil | VI |
| PI 628835 | FT-17 (Bandeirantes) | Brazil | VII |
| PI 628838 | FT-Abyara | Brazil | VII |
| PI 628842 | IAC-1 | Brazil | VIII |
| PI 628845 | IAC-10 | Brazil | VII |
| PI 628932 | FT-2 | Brazil | VII |
| PI 628936 | FT-Star | Brazil | VII |
| PI 632654 | VG 4763 | Vietnam | V |
| PI 307889 F | — | India | IX |
| PI 307891 B | — | India | IX |
| PI 594760 B | (Gou jiao huang dou) | China | IX |
| PI 628946 | IAC-8 | Brazil | IX |
| PI 614088 | Loda | United States | II |
| PI 548591 | Logan | United States | III |
| PI 593258 | Macon | United States | III |
| PI 548520 | Preston | United States | II |
| PI 548415 | Sooty | China | IV |
| PI 548619 | Sparks | United States | IV |
| PI 548645 | Pharaoh | United States | IV |
| PI 548614 | Sherman | United States | III |
| PI 608438 | Titan | United States | I |
| PI 546052 | OT89-14 | Canada | ZZZZ |
| PI 547694 | L65-756 | United States | III |
| PI 547788 | L82-1449 | United States | II |
| PI 546044 | OT89-06 | Canada | ZZ |
| PI 547841 | L77-1727 | United States | III |
| PI 548237 | T260H | United States | VII |
| PI 548256 | T279 | United States | VII |
| PI 642055 | DT97-4290 | United States | IV |
| PI 548988 | Pickett | United States | VI |
| PI 200538 | Sugao Zairai | Japan | VIII |
| PI 567767 B | (Tong shan da bai pi) | China | IV |
| PI 424610 | KAS 681-23 | South Korea | IV |
| PI 445837 | Violet | Romania | I |
| PI 89059 | 6063 | China | II |
| PI 437847 B | (DV-1532) | China | I |
| PI 92660 | 7855 | China | II |
| PI 92600 | 7795 | China | III |
| PI 70519 | 8310 | China | III |
| PI 438205 | VIR4491 | China | I |
| PI 90576-1 | 6486 | China | III |
| PI 70528 | 8370 | China | III |
| PI 88289 | 235 | China | III |
| PI 378665 | — | Hungary | Z |
| PI 92683 | 7878 | China | II |
| PI 251586 | Zagrebacka Rana | Bosnia and Herzegovina | I |
| PI 361072 | Gaterslebener St. 22 | Germany | ZZ |
| PI 68722 | 103 | China | Z |
| PI 89012 | 5957 | China | II |
| PI 92623 | 7818 | China | III |
| PI 437666 | I-vo-phyn | China | I |
| PI 70229 | 8021 | China | IV |
| PI 290131 | Locale 11 | Hungary | Z |
| PI 88351 | Selection No. 3 | China | II |
| PI 154189 | No. 57 | Netherlands | Z |
| PI 91120-3 | 6575 | China | III |
| PI 189945 | C F | France | I |
| PI 398342 | KAS 200-11 | South Korea | IV |
| PI 404155 A | Primorskij 450 | Russia | ZZ |
| PI 360955 A | Fiskeby V | Sweden | ZZZZ |
| PI 89772 | 7193 | China | IV |
| PI 154197 | No. 701 | Netherlands | ZZ |
| PI 372424 | Sesiles Novoslachtenie | Czech Republic | Z |
| PI 291313 | — | China | Z |
| PI 297548 | Ta chin hu houan tsa | China | I |
| PI 467312 | Cha-mo-shi-dou | China | II |
| PI 243529 | Goyo | Japan | IV |
| PI 378674 A | Pavlikeni 519 | Bulgaria | Z |
| PI 91732-1 | Grade No. 2 | China | I |
| PI 103091 | Wu An | China | IV |
| PI 438335 | SAO 196-C | Algeria | III |
| PI 291320 A | — | China | I |
| PI 399119 | — | South Korea | IV |
| PI 54620-2 | No. 60 | China | III |
| PI 92728 | 7923 | China | III |
| PI 95769 | 64 | South Korea | IV |
| PI 603176 A | — | North Korea | IV |
| FC 29219 | — | AT | II |
| PI 398994 | KLS 724-1 | South Korea | IV |
| PI 567541B | (Gun li huang) | China | III |
| PI 151249 | Soybean Brun Hatif U486 | Belgium | ZZ |
| PI 417246 | Rankoshi | Japan | II |
| PI 407715 | Jin nung No. 2 | China | I |
| PI 297502 | Cina 496-079 | China | I |
| PI 204653 | Strengs Weihenstephaner Schwarze | Germany | I |
| PI 561331 | Jiao he xiao hei dou | China | I |
| PI 468915 | — | China | II |
| PI 398739 | KAS 331-7 | South Korea | IV |
| PI 56563 | — | unknown | IV |
| PI 153214 | B-17 | Belgium | I |
| PI 408052 A | KAS 575-10 | South Korea | III |
| PI 567543 C | (He nan chun) | China | III |
| PI 132214 | No. D. 47 | Netherlands | ZZ |
| PI 391589 A | Hei nung No. 11 | China | I |
| PI 194630 | 698-3-5 | Sweden | ZZ |
| PI 417170 | Mutsu mejiro | Japan | II |
| PI 567374 | Ba yue zha | China | IV |
| PI 200471 | Hanayome Ibaragi No. 1 | Japan | III |
| PI 398644 | KAS 390-23 | South Korea | IV |
| PI 153225 | B-29 | Belgium | ZZ |
| PI 243548 | Uma-daizu | Japan | IV |
| PI 407788 A | ORD 8113 | South Korea | IV |
| PI 379559 C | (Komagi dadacha) | Japan | III |
| PI 574477 | Fen dou 31 | China | IV |
| PI 407949 | KAS 502-2 | South Korea | IV |
| PI 358321 A | — | China | ZZ |
| PI 603175 | GL 2688/96 | North Korea | IV |
| PI 153263 | Roumanie | Belgium | I |
| PI 132206 | No. D. 7 | Netherlands | I |
| PI 189946 | Tubingen | France | I |
| PI 291326 | — | China | ZZ |
| FC 30685 | Cha Kura Kake | Japan | ZZ |
| PI 153221 | Cha Kura Kake | Belgium | ZZ |
| PI 253651 B | No. 2 | China | IV |
| PI 153271 | Wisconsin Black | Belgium | I |
| PI 360955 B | (Fiskeby V) | Sweden | ZZZZ |
| PI 153223 | Ras 20 | Netherlands | ZZ |
| PI 417529 | A38 | Germany | Z |
| PI 205085 | I-Higo-Wase | Japan | I |
| PI 152361 | Hybrid No. 398-97 | Sweden | Z |
| PI 194648 | 751-3 | Sweden | ZZ |
| PI 253666 A | No. 17 | China | IV |
| PI 567519 | Bai hua chi | China | III |
| PI 417218 | Oomedama | Japan | II |
| PI 567354 | You huang dou | China | IV |
| PI 209332 | No. 4 | Japan | IV |
| PI 79691-4 | — | China | III |
| PI 81764 | Moshito | China | IV |
| PI 404166 | Krasnoarmej skaj a | Russia | III |
| PI 90575 | 6485 | China | II |
| PI 229343 | Nonaka No. 1 | Japan | IV |
| PI 153285 | N-26 | unknown | I |
| PI 79593 | N265/100 | China | II |
| PI 458515 | Tie Zhugan | China | IV |
| PI 567324 | Huang dou | China | IV |
| PI 417517 | Novosadska White | Yugoslavia | I |
| PI 194632 | 699-2-4 | Sweden | ZZ |
| PI 196502 | 634-20-4-29 | Sweden | ZZZZ |
| PI 507531 | Waseshu (2) | Japan | II |
| PI 417015 | Kawanagare (Iwate) | Japan | III |
| PI 342619 A | — | Russia | Z |
| PI 361057 | Berkners Gescheckte | Germany | I |
| PI 404198 B | (Sun huan do) | China | IV |
| PI 416904 C | (Hakubi) | China | I |
| PI 92706 | 7901 | China | I |
| PI 399020 | KLS 805-1 | South Korea | IV |
| PI 437725 | Te-zu-gan | China | IV |
| PI 567387 | Huang huai dou | China | IV |
| PI 153319 | Tohang | France | Z |
| PI 189876 | Weka | France | Z |
| PI 424078 | 74077 | South Korea | III |
| PI 567305 | Hei dou zi | China | IV |
| PI 81765 | Moshito | China | I |
| PI 194639 | 741-1 | Sweden | ZZZZ |
| PI 438497 | Peking | United States | III |
| PI 424159 B | KAS 643-8 | South Korea | IV |
| PI 81770 | Selection No. 503 | China | II |
| PI 135590 | No. 68-A | China | II |
| PI 407832 B | — | South Korea | IV |
| PI 68666 | 23 | China | II |
| PI 417140 | Masshokutou roshiyashu | Japan | II |
| PI 81766 | Moshito | China | III |
| PI 594403 | 85-125-1 | China | IV |
| PI 567537 | Gu li hun | China | II |
| PI 437654 | Er-hej-jan | China | III |
| PI 81773 | Shirosaya | Japan | II |
| PI 567719 | Fu yang (43) | China | IV |
| PI 567611 | Ba yue zha | China | IV |
| PI 438471 | Fiskeby III | Sweden | ZZ |
| PI 398637 | KAS 390-18 | South Korea | III |
| PI 326580 | — | Germany | I |
| PI 408124 B | KAS 638-5 | South Korea | IV |
| PI 189859 | Light Brown | France | Z |
| PI 361089 | Mittelfruheschwarze I | Germany | I |
| PI 561345 | Yi tong lu da dou | China | I |
| PI 189950 | Cosse Lisse | France | Z |
| PI 542044 | Kunitz | United States | III |
| PI 591507 | L89-1541 | United States | III |
| PI 591512 | L93-3258 | United States | III |
| PI 548636 | Regal | United States | IV |
| PI 547862 | L83-570 | United States | III |
| PI 548555 | Douglas | United States | IV |
| PI 547832 | L74-01 | United States | III |
| PI 591510 | L92-7857 | United States | III |
| PI 547488 | L67-3207 | United States | IV |
| PI 560206 | Delsoy 4210 | United States | IV |
| PI 547864 | L83-4494 | United States | III |
| PI 518674 | Fayette | United States | III |
| PI 548542 | Cumberland | United States | III |
| PI 518673 | Lawrence | United States | IV |
| PI 548522 | BSR 301 | United States | III |
| PI 548565 | Gnome | United States | II |
| PI 548635 | Chamberlain | United States | III |
| PI 597386 | Dwight | United States | II |
| PI 547651 | L80-5882 | United States | II |
| PI 548634 | Zane | United States | III |
| PI 612736 | Yi No. 3 | China | I |
| PI 540555 | Hamilton | United States | IV |
| PI 591488 | L91-8060 | United States | IV |
| PI 518668 | TN 4-86 | United States | IV |
| PI 548558 | Harper | United States | III |
| PI 548566 | Nebsoy | United States | II |
| PI 548521 | BSR 201 | United States | II |
| PI 548569 | Hack | United States | II |
| PI 540556 | Jack | United States | II |
| PI 542710 | Chapman | United States | II |
| PI 548633 | Wye | United States | IV |
| PI 543794 | Delsoy 4900 | United States | IV |
| PI 548563 | Franklin | United States | IV |
| PI 548632 | Woodworth | United States | III |
| PI 599299 | Stride | United States | I |
| PI 546487 | Archer | United States | I |
| PI 578335 B | (Perla 25) | Argentina | V |
| PI 612763 | MN1801 | United States | I |
| PI 557011 | Leslie | United States | I |
| PI 371610 | — | Pakistan | V |
| PI 540554 | Bell | United States | I |
| PI 548391 | Mukden | China | II |
| PI 548622 | Union | United States | IV |
| PI 548602 | Oksoy | United States | IV |
| PI 548616 | Sloan | United States | II |
| PI 548652 | Bass | United States | III |
| PI 547533 | L71-920 | United States | II |
| PI 542768 | Sturdy | United States | II |
| PI 595754 | Nemaha | United States | III |
| PI 548597 | Mead | United States | III |
| PI 548536 | Coles | United States | I |
| PI 567785 | OAC Shire | Canada | I |
| PI 548525 | BSR 302 | United States | III |
| PI 548571 | Harlon | Canada | I |
| PI 548573 | Harosoy | Canada | II |
| PI 548527 | Calland | United States | III |
| PI 647961 | R01-581F | United States | V |
| PI 96089 | 384 | North Korea | VI |
| PI 596414 | Clifford | United States | V |
| PI 615582 | CAVINESS | United States | V |
| PI 371612 | — | Pakistan | V |
| PI 548537 | Marion | United States | II |
| PI 548658 | Lee 74 | United States | VI |
| PI 593653 | Crowley | United States | V |
| PI 628879 | Parana | Brazil | V |
| PI 572239 | Holladay | United States | V |
| PI 584506 | Carver | United States | VII |
| PI 632668 | H 10 | Vietnam | VI |
| PI 547687 | L62-973 | United States | II |
| PI 576440 | Calhoun | United States | IV |
| PI 407961-1 | KAERI 503-10 | South Korea | V |
| PI 628812 | MG/BR-46 (Conquista) | Brazil | VI |
| PI 407957 | KAERI 503-6 | South Korea | V |
| PI 547472 | L65-774 | United States | II |
| PI 417392 | Tora mame | Japan | V |
| PI 561702 | Harbar | Mexico | VI |
| PI 230977 | No. 10 | Japan | VII |
| PI 548479 | Otootan | Taiwan | VIII |
| PI 628910 | BR-23 | Brazil | V |
| PI 170891 | 6/41S31 | South Africa | VI |
| PI 381666 | Kakira 9 | Uganda | V |
| PI 330635 | 50 S 136 | South Africa | VII |
| PI 170890 | 5/40S35 | South Africa | VI |
| PI 578247 | D85-10412 | United States | VI |
| PI 566971 A | MARIF 2517 | Indonesia | VIII |
| PI 632663 B | (H 5) | Vietnam | V |
| PI 398481 | KAS 230-6 | South Korea | V |
| PI 548613 | Scott | United States | IV |
| PI 553039 | Davis | United States | VI |
| PI 598358 | TN 5-95 | United States | V |
| PI 635039 | S99-3181 | United States | V |
| PI 506947 | Kumaji 2 | Japan | VIII |
| PI 408045 | KAERI 575-3 | South Korea | V |
| PI 587829 | E huang No. 9 | China | VII |
| PI 499955 | — | China | VII |
| PI 511813 | Twiggs | United States | VI |
| PI 148260 | Potchefstroom | South Africa | VI |
| PI 594541 | Ming qiu No. 3 | China | VII |
| PI 567070 A | MARIF 2668 | Indonesia | VIII |
| PI 578332 B | (OFPEC Income 801) | Argentina | VII |
| PI 398423 | KAS 201-9 | South Korea | V |
| PI 459025 B | (Bing nan) | China | VIII |
| PI 594512 A | Bian zi jiang se dou | China | VII |
| PI 307882 E | No. 47 | India | IX |
| PI 398608 | KAS 390-8 | South Korea | V |
| PI 605839 B | (Sham si man) | Vietnam | V |
| PI 398438 | KAS 205-10 | South Korea | V |
| PI 567521 | Bai jia | China | V |
| PI 80468 | Tsurunoko Daizu | Japan | VI |
| PI 339863 A | Dongsan No. 6 | South Korea | V |
| PI 398316 | KAS 181-2 | South Korea | V |
| PI 398962 | KLS 625 | South Korea | V |
| PI 594887 | Yang yan dou | China | V |
| PI 417130 | Kyushu 47 | Japan | VIII |
| PI 408011 | KAERI 548-4 | South Korea | V |
| PI 398918 | KLS 304 | South Korea | V |
| PI 548483 | Pocahontas | unknown | VII |
| PI 374176 | U-4 | India | VIII |
| PI 408040-1 | KAERI 572-3 | South Korea | V |
| PI 588014 C | (Da bai mao) | China | VII |
| PI 602593 | MN1301 | United States | I |
| PI 339982 | No. 6 | South Korea | V |
| PI 203400 | White of the Rio Grande | France | VIII |
| PI 417499 | Aratiba | Brazil | IX |
| PI 175175 | No. 9434-A | India | VIII |
| PI 341261 | HLS 239 | Tanzania | IX |
| PI 428692 | — | India | IX |
| PI 588000 | Shi yue huang | China | X |
| PI 628824 | FT-5 (Formosa) | Brazil | VIII |
| PI 398219 | KAS 102-5-2 | South Korea | V |
| PI 594885 B | (Song zi dou) | China | VII |
| PI 157476 | Sun-cheon | South Korea | VI |
| PI 587627 B | (Hai men guan qing dou) | China | VII |
| PI 200503 | Miyashiro jun | Japan | V |
| PI 408340 | KAERI 590-4 | South Korea | VI |
| PI 379622 | P 156 | Taiwan | VI |
| PI 417206 | Oho Mame | Japan | VII |
| PI 324068 | Hernon 273 | Zimbabwe | VIII |
| PI 417208 | Oka Kaizu | Japan | VIII |
| PI 471938 | 197 | Nepal | V |
| PI 200546 | Wada ani | Japan | V |
| PI 417369 | Tamana | Japan | VIII |
| PI 567025 A | MARIF 2592 | Indonesia | VIII |
| PI 200492 | Komata | Japan | VII |
| PI 567095 A | MARIF 2693 | Indonesia | VIII |
| PI 189402 | 55-50 | Guatemala | VIII |
| PI 209333 | No. 3 | Japan | VI |
| PI 407962-2 | KAERI 504-1 | South Korea | V |
| PI 417063 | Kotane | Japan | VII |
| PI 215755 | Soya Otootan | Peru | VIII |
| PI 417136 | Manshuu Konpo Daizu | Japan | VIII |
| PI 459025 A | Bing nan | China | IX |
| PI 632666 | H 8 | Vietnam | V |
| PI 567020 A | MARIF 2587 | Indonesia | VIII |
| PI 417215 | Ooita Aki Daizu 2 | Japan | VIII |
| PI 507301 | Souta Daizu | Japan | VIII |
| PI 567054 C | MARIF 2647 | Indonesia | IX |
| PI 628825 | FT-6 (Venice) | Brazil | VIII |
| PI 374169 | I-7 | India | VIII |
| PI 567129 | MARIF 2796 | Indonesia | IX |
| PI 587916 A | Da qing dou | China | IX |
| PI 219789 | Shin No. 4 | Japan | V |
| PI 438426 | VIR 5530 | India | VI |
| PI 247679 | Otootan | Zaire | VIII |
| PI 561271 | Pei xian da quing dou | China | V |
| PI 567399 | Niu mao huang | China | V |
| PI 208437 | No. 9 | Nepal | VII |
| PI 398828 | KAS 360-14 | South Korea | V |
| PI 164885 | No. 15 Seed black | Guatemala | VIII |
| PI 407790-2 | ORD 8118 | South Korea | V |
| PI 407990 | KAERI 542-9 | South Korea | V |
| PI 567070 B | MARIF 2668 | Indonesia | VIII |
| PI 507006 | Kyuushuu 38 | Japan | VI |
| PI 417472 D | (Yatsufusa) | Japan | V |
| PI 567088 A | MARIF 2686 | Indonesia | VIII |
| PI 567053 | MARIF 2635 | Indonesia | IX |
| PI 628886 | RS-6 (Guassupi) | Brazil | VII |
| PI 374182 | D-4 | India | VIII |
| PI 374183 | D-5 | India | VIII |
| PI 374171 | I-9 | India | VIII |
| PI 408049 | KAERI 575-7 | South Korea | V |
| PI 567077 B | MARIF 2675 | Indonesia | IX |
| PI 567073 B | MARIF 2671 | Indonesia | VIII |
| PI 594538 B | (Min hou bai sha wan dou) | China | VIII |
| PI 594591 B | (Sui ning ba yue huang (jia)) | China | VI |
| PI 374186 | SM-2 | India | VIII |
| PI 417061 | Kosa Mame | Japan | VIII |
| PI 497966 | PLSO 55 | India | VI |
| PI 408003-2 | KAERI 544-9 | South Korea | VI |
| PI 548359 | Kingwa | China | IV |
| PI 567088 B | MARIF 2686 | Indonesia | VIII |
| PI 567136 A | MARIF 2803 | Indonesia | VIII |
| PI 200474 | Hikage Daizu | Japan | VIII |
| PI 200487 | Kinoshita | Japan | VIII |
| PI 567063 | MARIF 2661 | Indonesia | VII |
| PI 615510 B | (Hat to 2 vu te nau) | Vietnam | V |
| PI 208783 | Kaikon-Mame | Japan | VII |
| PI 229358 | Soden-daizu | Japan | VII |
| PI 416828 | Chiba nouken 3 | Japan | VIII |
| PI 567039 | MARIF 2618 | Indonesia | VII |
| PI 567091 | MARIF 2689 | Indonesia | VIII |
| PI 612611 | Browngilgun | North Korea | III |
| PI 547521 | L70-4190 | United States | IV |
| PI 374166 | I-4 | India | VIII |
| PI 506694 | Gioo | Japan | V |
| PI 548402 | Peking | China | IV |
| PI 219656 | Reg. No. 520 | Indonesia | VI |
| PI 567068 A | MARIF 2666 | Indonesia | VII |
| PI 632663 A | H 5 | Vietnam | V |
| PI 567270 C | (Local mixed) | China | V |
| PI 175177 | No. 9577-A | Nepal | VIII |
| PI 374158 | M-5 | India | VIII |
| PI 200451 | Amakusa Daizu | Japan | VIII |
| PI 393546 | — | Taiwan | VIII |
| PI 543793 | Delsoy 4500 | United States | IV |
| PI 588023 A | Gao shan huang dou | China | VII |
| PI 632935 B | (Vang ninh tap) | Vietnam | V |
| PI 205899 | Laheng | Thailand | VIII |
| PI 259542 | Preta da Estacao | Angola | IX |
| PI 307853 | No. 18 | India | IX |
| PI 548667 | Essex | United States | V |
| PI 471904 | Orba | Indonesia | IX |
| PI 407757 | 43130 | China | V |
| PI 471940 | 240 | Nepal | VI |
| PI 203403 | New Granada | Japan | VIII |
| PI 240665 | Black Manchurian | Philippines | VIII |
| PI 374157 | M-4 | India | VIII |
| PI 587880 A | Huang dou | China | VI |
| PI 603527 B | (Hei liao dou) | China | V |
| PI 567089 A | MARIF 2687 | Indonesia | VIII |
| PI 548557 | Elgin | United States | II |
| PI 605869 A | Sample 140 | Vietnam | V |
| PI 407978 | KAERI 541-3 | South Korea | V |
| PI 587867 | Jiu yue huang | China | VII |
| PI 587814 D | (Ba yue dou) | China | VII |
| PI 587560 A | Dan tu ba yue bai jia | China | VII |
| PI 587573 B | (Yi xing zhong zi dou yi) | China | VII |
| PI 212604 | No. 13100 | Afghanistan | VI |
| PI 628832 | FT-14 (Piracema) | Brazil | IX |
| PI 407930 | KAS 552-2 | South Korea | V |
| PI 340000 | Jongsun | South Korea | V |
| PI 326578 | K-5363 | China | VIII |
| PI 331793 | Dia-Phyng | Vietnam | VIII |
| PI 307597 | Bhatwans | India | IX |
| Pintado | BRSMT Pintado | Brazil | VIII |
| Conquista | Conquista | Brazil | VIII |
| BRSGO | BRSGO Chapadoes | Brazil | VIII |
| Chapadoes PI | I-3 | India | VIII |
| 374165 | | | |
| PI 578335 A | Pearl 25 | Argentina | V |
| PI 175198 | No. 10294 | India | VI |
| PI 578478 B | (Huai 823) | China | V |
| PI 240664 | Bilomi No. 3 | Philippines | X |
| PI 632748 | VS94-12 | United States | VI |
| PI 587950 | Sha xian wu dou | China | IX |
| PI 561356 | Jin yun dou | China | V |
| PI 238109 | Jugatsu Shiromame | Japan | X |
| PI 175176 | No. 9446-A | India | VIII |
| PI 603608 | Huang pi shan zi bai | China | VII |
| PI 548606 | Pomona | United States | IV |
| PI 274453 | — | Japan | X |
| PI 548646 | RCAT Alliance | Canada | II |
| PI 306704 A | 7H/101 | Kenya | IX |
| PI 587568 A | Li yang xiao zi da dou | China | VII |
| PI 262180 | Sankuo | Japan | VIII |
| PI 374168 | I-6 | India | VIII |
| PI 587709 A | Chong ming shi yue huang | China | VII |
| PI 547791 | L85-129 | United States | II |
| PI 307889 B | No. 54 | India | IX |
| PI 597388 | Accomac | United States | V |
| PI 417009 | Karasumame (Naihou) | Japan | VIII |
| PI 567121 A | MARIF 2788 | Indonesia | VIII |
| PI 594538 A | Min hou bai sha wan dou | China | IX |
| PI 542709 | Hayes | United States | III |
| PI 594698 | Huang dou 13 | China | V |
| PI 598124 | Maverick | United States | III |
| PI 603605 | Jing 225 | China | VII |
| PI 416873 B | (Fusanari daizu) | Japan | VIII |
| PI 175181 | No. 10002 | India | VII |
| PI 594834 B | (Wu yue bai dou) | China | VII |
| PI 594668 | Huang dou zi | China | V |
| PI 605887 C | — | Vietnam | VI |
| PI 506500 | Akasaya (Mejiro) | Japan | VI |
| PI 605832 A | Sample 97 | Vietnam | V |
| PI 587992 E | (Jiu yue huang) | China | VII |
| PI 548631 | Williams | United States | III |
| PI 239236 | OtootanNo. 6 | Thailand | IX |
| PI 200526 | Shira Nuhi | Japan | VIII |
| PI 591511 | L89-1581 | United States | III |
| PI 548977 | Epps | United States | V |
| PI 567056 A | MARIF 2649 | Indonesia | VIII |
| PI 587878 | Shang tian huang | China | VII |
| PI 417445 | Wase cha shouryuu | Japan | V |
| PI 586981 | KS4694 | United States | IV |
| PI 587984 A | Bai shui dou | China | V |
| PI 393565 B | — | Thailand | VIII |
| PI 222550 | 951-DCE-Sj-096 | Argentina | VIII |
| PI 547818 | L74-142 | United States | III |
| PI 587828 | Xiang yang qing dou | China | VII |
| PI 539864 | HP203 | United States | I |
| PI 240671 | Yellow Biloxi 37 | Philippines | VIII |
| PI 603154 | GL 2622/96 | North Korea | V |
| PI 468967 | 86 | Vietnam | V |
| PI 605792 C | Sample 56 | Vietnam | V |
| PI 567378 | Ba yue zha | China | VI |
| PI 222549 | 951-DCE-Sj-094 | Argentina | IX |
| PI 408330 | KAERI 646-4 | South Korea | V |
| PI 594548 | Heng feng gui zi dou | China | VII |
| PI 594667 | Jiang kou huang dou No. 4 | China | V |
| PI 548445 | CNS | China | VII |
| PI 567230 | WJK-PRC-23 | China | V |
| PI 408056 | KAERI 576-4 | South Korea | V |
| PI 594707 | Da hei dou | China | VII |
| PI 510670 | Morgan | United States | IV |
| PI 374178 | U-6 | India | VIII |
| PI 591432 | OT94-51 | Canada | Z |
| PI 572240 | Nile | United States | IV |
| PI 374160 | M-7 | India | VIII |
| PI 417120 | Kyushu 25 | Japan | VIII |
| PI 339869 | Ajukarikong | South Korea | V |
| PI 594480 C | (Lu dou) | China | VII |
| PI 81027 | Akasaya Daizu | Japan | AT |
| PI 157492 | Yuc-u No. 7 | Japan | AT |
| PI 567190 | Halang 4 thang | Vietnam | AT |
| PI 86876 | Daizu Pikuanda | Japan | IV |
| PI 88294-1 | 5683 | China | II |
| PI 567078 | MARIF 2676 | Indonesia | VII |
| PI 560207 | Delsoy 4710 | United States | IV |
| PI 515961 | Pennyrile | United States | IV |
| PI 635999 | DT2000 | Vietnam | VI |
| PI 424405 B | KAS 530-16 | South Korea | IV |
| PI 92595 | 7790 | China | II |
| PI 383277 | Jilin No. 5 | China | II |
| PI 578368 | 164-4-32 | China | III |
| PI 297542 | Pannonia 10 | Hungary | Z |
| PI 407706 A | Chou yao tao | China | I |
| PI 70520 | 8312 | China | I |
| PI 437660 | Gun 246 | China | Z |
| PI 88826 | Kurugara | Japan | III |
| PI 84664 | S-92 | South Korea | IV |
| PI 89070 | 6067 | China | II |
| PI 189967 | V 6 | France | I |
| PI 153234 | J-5A | Netherlands | ZZ |
| PI 257433 | C 15/58 | Germany | Z |
| PI 253655 | No. 6 | China | IV |
| PI 361071 C | (Gaterslebener St. 7) | Germany | I |
| PI 323556 | H 67-7 | India | IV |
| PI 398682 | KAS 320-3 | South Korea | IV |
| PI 153290 | Altonagaard A1 | Denmark | I |
| PI 603501 | Lu pi da dou | China | IV |
| PI 603497 | Hua dou | China | III |
| PI 295949 | Amurskaja 266 | Russia | Z |
| PI 295947 | Amurskaja 57 | Russia | Z |
| PI 361110 | Secca | Romania | ZZZZ |
| PI 398313 | KAS 180-5 | South Korea | IV |
| PI 189861 | Grignon 18 | France | Z |
| PI 547838 | L76-1988 | United States | III |
| PI 548541 | Crawford | United States | IV |
| PI 542043 | Linford | United States | III |
| PI 548549 | DeSoto | United States | IV |
| PI 548585 | Winchester | United States | III |
| PI 548538 | Columbus | United States | IV |
| PI 547589 | L63-3270 | United States | III |
| PI 595363 | Mustang | United States | IV |
| PI 612738 | 67803 | China | I |
| PI 599300 | Appears | United States | Z |
| PI 592524 | Granite | United States | I |
| PI 562373 | Lambert | United States | Z |
| PI 612764 | MN0901 | United States | Z |
| PI 629005 | MN0302 | United States | Z |
| PI 594822 | Xi huang dou | China | IX |
| PI 417261 | Saishuutou Tansei Zairai | Japan | VIII |
| PI 407983 | KAERI 542-3-1 | South Korea | V |
| PI 374154 | M-1 | India | VIII |
| PI 628847 | IAC-12 | Brazil | VII |
| PI 561359 | I give | China | VIII |
| PI 174867 | No. 10303 | India | VIII |
| PI 605879 | Dau lu | Vietnam | V |
| PI 632665 | H 7 | Vietnam | IV |
| PI 632639 D | (Hoang mao) | Vietnam | V |
| PI 605853 B | (From trui) | Vietnam | V |
| PI 434974 | Seminole | China | IX |
| PI 587871 | Bao mao dou | China | VII |
| PI 434980 A | Going 180 | Central African Republic | VIII |
| PI 208435 | No. 7 Mixed | Nepal | VIII |
| PI 605824 A | Sample 88 | Vietnam | V |
| PI 606389 | Doan ket | Vietnam | V |
| PI 548543 | Oakland | United States | III |
| PI 562374 | Parker | United States | I |
| PI 658519 | LD00-2817P | AT | AT |
| PI 381657 | 3H55 F4/9/2 | Uganda | VIII |

### Example 2

### Isolates of Corynespora cassiicola

Seventeen isolates of Corynespora cassiicola were selected from the Holder's mycoteca that showed virulence considered high and intermediate, obtained in studies conducted on the Holder's premises. The isolates are described in Table 2.

TABLE 2
Corynespora cassiicola isolates used in this work.

| Code Isolated¹ | Source | Culture | Virulência2 |
|---|---|---|---|
| TMG 080 | Sapezal, MT | Soy | +++ |
| TMG 082 | Porto dos Gaúcos, MT | Soy | +++ |
| TMG 083 | Nova Mutum, MT | Soy | +++ |
| TMG 109 | Sorriso, MT | Soy | +++ |
| TMG 116 | Guarai, TO | Soy | +++ |
| TMG 119 | Correntina, BA | Soy | +++ |
| FMT 050 | Sorriso, MT | Soy | +++ |
| TMG 069 | Montividiu, GO | Soy | ++ |
| TMG 106 | Matupá, MT | Soy | ++ |
| TMG 107 | Matupá, MT | Soy | ++ |
| TMG 110 | Confresa, MT | Soy | ++ |
| TMG115 | Correntina, BA | Soy | ++ |
| TMG 118 | Silvanópolis, TO | Soy | ++ |
| FMT 051 | Itiquira, MT | Soy | ++ |
| FMT 060 | Rondonopolis, MT | Cotton | ++ |
| 24 (Cory 6.1) | Rondonopolis, MT | Soy | ++ |
| 34 (Cory 9.1) | Rondonopolis, MT | Soy | ++ |

¹Isolates preserved at Castelani;
2Obtained by pathogenicity test in work conducted at TMG: +++ (represents high virulence), ++ (represents intermediate virulence).

Pure cultures of the fungi were obtained on BDA medium (potato-dextrose-agar) for 7 days. A repetition of each isolate was taken from the plate and mixed in a container, adding 100 mL of water, and proceeding with grinding in a blender for about 30 s. The solution obtained was filtered through a 20-mesh sieve. The residue that was retained on the sieve was discarded, and an aliquot was taken from the conidia suspension mix to count the spores. The final spore count of the suspension was 1750 conidia/mL.

### Example 3

### Phenotypic Evaluation

The materials selected for this study were planted in the greenhouse to evaluate disease resistance, with a total of four samples per genotype. Two months after planting, the genotypes were inoculated with the bulk of the 17 Corynespora cassiicola isolates. Initially, twenty liters of spore suspension were prepared and sprayed with the aid of a backpack pump over the leaf area of the plants. Two inoculations were carried out, with an interval of 5 days. The inoculations were performed in the late afternoon, with leaf wetting on the five days following inoculations.

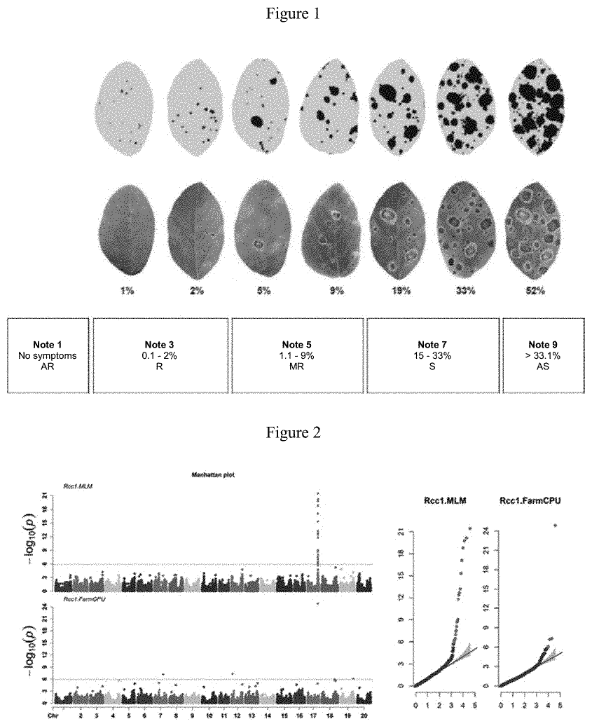

As a way to evaluate the disease response, two assessments were performed. First the average severity score was evaluated. For this, we used the diagrammatic scale developed by Soares and collaborators (2009) (SOARES, R. M.; GODOY, C. V.; OLIVEIRA, M. C. N. Diagrammatic scale for assessing the severity of target spot of soybean. Tropical Plant Pathology, v.34, p. 333-338, 2009) with some modifications (FIG. 1). In addition to this, lesion size was also observed and grades from 1-5 were assigned, visually, to the diameter of the lesions.

After the two evaluations, the genotypes with Highly Resistant/Immune reaction were selected (AR) or Resistance (R) the target spot and with lesion size ranging from 0 to 2 mm for a new planting. The purpose of this new evaluation was to confirm the resistance or whether there was any leakage during the test. To that end, ten seeds of each genotype were planted in 8 L pots containing soil:sand, in a 3:1 ratio. As susceptible standard we used the cultivar NA 5909 and some genotypes with Susceptible reaction (S) or Highly Susceptible (AS) of the first trial. Again a spore suspension was prepared with spore count/mL and proceeded with spraying/first inoculation, in the greenhouse at the V2 stage.

The second inoculation occurred 4 days after the first inoculation. Inoculations were performed in the late afternoon, and leaf wetting was maintained for five days after inoculations. The evaluation was performed 20 days after the last inoculation, by determining the average severity score and lesion size (Table 3).

TABLE 3
Scale of scores for the evaluation of the severity of target spot in soybean leaf tissue

| Note | Severity (%) | Reaction | Lesion size (mm) |
|---|---|---|---|
| 1 | 0% | AR | 0 |
| 2 | 1-10% | R | 1-2 |
| 3 | 11-20% | MR | 3-4 |
| 4 | 21-40% | S | 4-5 |
| 5 | >40% | AS | >5 |

A total of 83 genotypes showed resistance to the action of the pathogen. Of these, seven materials were highly resistant to target spot: PI 71506, PI 153230, PI 567310B, PI 587802, PI 587860, PI 407999-1 and PI 548984. These materials can be worked on in breeding programs as sources of resistance to the target spot. In contrast, 616 materials showed susceptibility to the disease, of which 67 were highly susceptible. The classification of the materials as to their resistance to target spot can be seen in Table 4.

TABLE 4

| Material | Phenotypic reaction |
|---|---|
| PI71506 | Highly Resistant |
| PI153230 | Highly Resistant |
| PI567310B | Highly Resistant |
| PI587802 | Highly Resistant |
| PI587860 | Highly Resistant |
| PI407999-1 | Highly Resistant |
| PI548984 | Highly Resistant |
| PI347550A | Resistant |
| PI417115 | Resistant |
| PI87606 | Resistant |
| PI319537A | Resistant |
| PI603572 | Resistant |
| PI594762 | Resistant |
| PI534646 | Resistant |
| PI576857 | Resistant |
| PI640911 | Resistant |
| PI424079 | Resistant |
| PI424611A | Resistant |
| PI424611B | Resistant |
| PI424612 | Resistant |
| PI84578 | Resistant |
| PI290116A | Resistant |
| PI248398 | Resistant |
| PI360954 | Resistant |
| PI258384 | Resistant |
| PI153274 | Resistant |
| PI209335 | Resistant |
| PI89002 | Resistant |
| PI407659B | Resistant |
| PI189872 | Resistant |
| PI495017C | Resistant |
| PI196148 | Resistant |
| PI90763 | Resistant |
| PI238925 | Resistant |
| PI360964 | Resistant |
| PI417095 | Resistant |
| PI507352 | Resistant |
| PI200480 | Resistant |
| PI297551 | Resistant |
| PI88788 | Resistant |
| PI194624 | Resistant |
| PI361085B | Resistant |
| PI567597C | Resistant |
| PI547842 | Resistant |
| PI547876 | Resistant |
| PI595843 | Resistant |
| PI548318 | Resistant |
| PI548524 | Resistant |
| PI548663 | Resistant |
| PI543855 | Resistant |
| PI546039 | Resistant |
| PI618613 | Resistant |
| PI398447 | Resistant |
| PI506624 | Resistant |
| PI548659 | Resistant |
| PI416806 | Resistant |
| PI408042 | Resistant |
| PI170889 | Resistant |
| PI594754 | Resistant |
| PI594527 | Resistant |
| PI567132C | Resistant |
| PI307882C | Resistant |
| PI587687E | Resistant |
| PI424442 | Resistant |
| PI229320 | Resistant |
| PI416937 | Resistant |
| PI593999A | Resistant |
| PI407987 | Resistant |
| PI567126 | Resistant |
| PI417011 | Resistant |
| PI567104B | Resistant |
| PI567076 | Resistant |
| PI567397 | Resistant |
| PI230979 | Resistant |
| PI559371 | Resistant |
| PI587883B | Resistant |
| PI587668B | Resistant |
| PI518722 | Resistant |
| PI587886 | Resistant |
| PI476918 | Resistant |
| PI632648 | Resistant |
| PI506764 | Resistant |
| PI561373 | Resistant |
| CD 201 | Susceptible |
| NA 5909 RG | Susceptible |
| Tapir 82 | Susceptible |
| Williams 82 | Susceptible |
| PI632667 | Susceptible |
| PI543832 | Susceptible |
| PI594675 | Susceptible |
| PI68494 | Susceptible |
| PI68621 | Susceptible |
| PI84580 | Susceptible |
| PI84957 | Susceptible |
| PI85626 | Susceptible |
| PI86102 | Susceptible |
| PI86972-1 | Susceptible |
| PI87531 | Susceptible |
| PI87617 | Susceptible |
| PI88508 | Susceptible |
| PI153311 | Susceptible |
| PI153313 | Susceptible |
| PI157431 | Susceptible |
| PI358313 | Susceptible |
| PI398735 | Susceptible |
| PI408132 | Susceptible |
| PI417524 | Susceptible |
| PI424298 | Susceptible |
| PI499957 | Susceptible |
| PI507354 | Susceptible |
| PI507686C | Susceptible |
| PI567651 | Susceptible |
| PI594599 | Susceptible |
| PI632661B | Susceptible |
| PI174862 | Susceptible |
| PI269518C | Susceptible |
| PI323564 | Susceptible |
| PI374162 | Susceptible |
| PI378693A | Susceptible |
| PI408046 | Susceptible |
| PI423913 | Susceptible |
| PI423966 | Susceptible |
| PI458122 | Susceptible |
| PI476905A | Susceptible |
| PI567079 | Susceptible |
| PI567082A | Susceptible |
| PI567346 | Susceptible |
| PI587905 | Susceptible |
| PI587996B | Susceptible |
| PI594669 | Susceptible |
| PI605779C | Susceptible |
| PI605779D | Susceptible |
| PI615487 | Susceptible |
| PI628803 | Susceptible |
| PI628835 | Susceptible |
| PI628838 | Susceptible |
| PI628842 | Susceptible |
| PI628845 | Susceptible |
| PI628932 | Susceptible |
| PI628936 | Susceptible |
| PI632654 | Susceptible |
| PI307889F | Susceptible |
| PI307891B | Susceptible |
| PI594760B | Susceptible |
| PI628946 | Susceptible |
| PI614088 | Susceptible |
| PI548591 | Susceptible |
| PI593258 | Susceptible |
| PI548520 | Susceptible |
| PI548415 | Susceptible |
| PI548619 | Susceptible |
| PI548645 | Susceptible |
| PI548614 | Susceptible |
| PI608438 | Susceptible |
| PI546052 | Susceptible |
| PI547694 | Susceptible |
| PI547788 | Susceptible |
| PI546044 | Susceptible |
| PI547841 | Susceptible |
| PI548237 | Susceptible |
| PI548256 | Susceptible |
| PI642055 | Susceptible |
| PI548988 | Susceptible |
| PI200538 | Susceptible |
| PI567767B | Susceptible |
| PI424610 | Susceptible |
| PI445837 | Susceptible |
| PI89059 | Susceptible |
| PI437847B | Susceptible |
| PI92660 | Susceptible |
| PI92600 | Susceptible |
| PI70519 | Susceptible |
| PI438205 | Susceptible |
| PI90576-1 | Susceptible |
| PI70528 | Susceptible |
| PI88289 | Susceptible |
| PI378665 | Susceptible |
| PI92683 | Susceptible |
| PI251586 | Susceptible |
| PI361072 | Susceptible |
| PK8722 | Susceptible |
| PI89012 | Susceptible |
| PI92623 | Susceptible |
| PI437666 | Susceptible |
| PI70229 | Susceptible |
| PI290131 | Susceptible |
| PI88351 | Susceptible |
| PI154189 | Susceptible |
| PI91120-3 | Susceptible |
| PI189945 | Susceptible |
| PI398342 | Susceptible |
| PI404155A | Susceptible |
| PI360955A | Susceptible |
| PI89772 | Susceptible |
| PI154197 | Susceptible |
| PI372424 | Susceptible |
| PI291313 | Susceptible |
| PI297548 | Susceptible |
| PI467312 | Susceptible |
| PI243529 | Susceptible |
| PI378674A | Susceptible |
| PI91732-1 | Susceptible |
| PI103091 | Susceptible |
| PI438335 | Susceptible |
| PI291320A | Susceptible |
| PI399119 | Susceptible |
| PI54620-2 | Susceptible |
| PI92728 | Susceptible |
| PI95769 | Susceptible |
| PI603176A | Susceptible |
| FC29219 | Susceptible |
| PI398994 | Susceptible |
| PI567541B | Susceptible |
| PI151249 | Susceptible |
| PI417246 | Susceptible |
| PI407715 | Susceptible |
| PI297502 | Susceptible |
| PI204653 | Susceptible |
| PI561331 | Susceptible |
| PI468915 | Susceptible |
| PI398739 | Susceptible |
| PI56563 | Susceptible |
| PI153214 | Susceptible |
| PI408052A | Susceptible |
| PI567543C | Susceptible |
| PI132214 | Susceptible |
| PI391589A | Susceptible |
| PI194630 | Susceptible |
| PI417170 | Susceptible |
| PI567374 | Susceptible |
| PI200471 | Susceptible |
| PI398644 | Susceptible |
| PI153225 | Susceptible |
| PI243548 | Susceptible |
| PI407788A | Susceptible |
| PI379559C | Susceptible |
| PI574477 | Susceptible |
| PI407949 | Susceptible |
| PI358321A | Susceptible |
| PI603175 | Susceptible |
| PI153263 | Susceptible |
| PI132206 | Susceptible |
| PI189946 | Susceptible |
| PI291326 | Susceptible |
| FC30685 | Susceptible |
| PI153221 | Susceptible |
| PI253651B | Susceptible |
| PI153271 | Susceptible |
| PI360955B | Susceptible |
| PI153223 | Susceptible |
| PI417529 | Susceptible |
| PI205085 | Susceptible |
| PI152361 | Susceptible |
| PI194648 | Susceptible |
| PI253666A | Susceptible |
| PI567519 | Susceptible |
| PI417218 | Susceptible |
| PI567354 | Susceptible |
| PI209332 | Susceptible |
| PI79691-4 | Susceptible |
| PI81764 | Susceptible |
| PI404166 | Susceptible |
| PI90575 | Susceptible |
| PI229343 | Susceptible |
| PI153285 | Susceptible |
| PI79593 | Susceptible |
| PI458515 | Susceptible |
| PI567324 | Susceptible |
| PI417517 | Susceptible |
| PI194632 | Susceptible |
| PI196502 | Susceptible |
| PI507531 | Susceptible |
| PI417015 | Susceptible |
| PI342619A | Susceptible |
| PI361057 | Susceptible |
| PI404198B | Susceptible |
| PI416904C | Susceptible |
| PI92706 | Susceptible |
| PI399020 | Susceptible |
| PI437725 | Susceptible |
| PI567387 | Susceptible |
| PI153319 | Susceptible |
| PI189876 | Susceptible |
| PI424078 | Susceptible |
| PI567305 | Susceptible |
| PI81765 | Susceptible |
| PI194639 | Susceptible |
| PI438497 | Susceptible |
| PI424159B | Susceptible |
| PI81770 | Susceptible |
| PI135590 | Susceptible |
| PI407832B | Susceptible |
| PI68666 | Susceptible |
| PI417140 | Susceptible |
| PI81766 | Susceptible |
| PI594403 | Susceptible |
| PI567537 | Susceptible |
| PI437654 | Susceptible |
| PI81773 | Susceptible |
| PI567719 | Susceptible |
| PI567611 | Susceptible |
| PI438471 | Susceptible |
| PI398637 | Susceptible |
| PI326580 | Susceptible |
| PI408124B | Susceptible |
| PI189859 | Susceptible |
| PI361089 | Susceptible |
| PI561345 | Susceptible |
| PI189950 | Susceptible |
| PI542044 | Susceptible |
| PI591507 | Susceptible |
| PI591512 | Susceptible |
| PI548636 | Susceptible |
| PI547862 | Susceptible |
| PI548555 | Susceptible |
| PI547832 | Susceptible |
| PI591510 | Susceptible |
| PI547488 | Susceptible |
| PI560206 | Susceptible |
| PI547864 | Susceptible |
| PI518674 | Susceptible |
| PI548542 | Susceptible |
| PI518673 | Susceptible |
| PI548522 | Susceptible |
| PI548565 | Susceptible |
| PI548635 | Susceptible |
| PI597386 | Susceptible |
| PI547651 | Susceptible |
| PI548634 | Susceptible |
| PI612736 | Susceptible |
| PI540555 | Susceptible |
| PI591488 | Susceptible |
| PI518668 | Susceptible |
| PI548558 | Susceptible |
| PI548566 | Susceptible |
| PI548521 | Susceptible |
| PI548569 | Susceptible |
| PI540556 | Susceptible |
| PI542710 | Susceptible |
| PI548633 | Susceptible |
| PI543794 | Susceptible |
| PI548563 | Susceptible |
| PI548632 | Susceptible |
| PI599299 | Susceptible |
| PI546487 | Susceptible |
| PI578335B | Susceptible |
| PI612763 | Susceptible |
| PI557011 | Susceptible |
| PI371610 | Susceptible |
| PI540554 | Susceptible |
| PI548391 | Susceptible |
| PI548622 | Susceptible |
| PI548602 | Susceptible |
| PI548616 | Susceptible |
| PI548652 | Susceptible |
| PI547533 | Susceptible |
| PI542768 | Susceptible |
| PI595754 | Susceptible |
| PI548597 | Susceptible |
| PI548536 | Susceptible |
| PI567785 | Susceptible |
| PI548525 | Susceptible |
| PI548571 | Susceptible |
| PI548573 | Susceptible |
| PI548527 | Susceptible |
| PI647961 | Susceptible |
| PI96089 | Susceptible |
| PI596414 | Susceptible |
| PI615582 | Susceptible |
| PI371612 | Susceptible |
| PI548537 | Susceptible |
| PI548658 | Susceptible |
| PI593653 | Susceptible |
| PI628879 | Susceptible |
| PI572239 | Susceptible |
| PI584506 | Susceptible |
| PI632668 | Susceptible |
| PI547687 | Susceptible |
| PI576440 | Susceptible |
| PI407961-1 | Susceptible |
| PI628812 | Susceptible |
| PI407957 | Susceptible |
| PI547472 | Susceptible |
| PI417392 | Susceptible |
| PI561702 | Susceptible |
| PI230977 | Susceptible |
| PI548479 | Susceptible |
| PI628910 | Susceptible |
| PI170891 | Susceptible |
| PI381666 | Susceptible |
| PI330635 | Susceptible |
| PI170890 | Susceptible |
| PI578247 | Susceptible |
| PI566971A | Susceptible |
| PI632663B | Susceptible |
| PI398481 | Susceptible |
| PI548613 | Susceptible |
| PI553039 | Susceptible |
| PI598358 | Susceptible |
| PI635039 | Susceptible |
| PI506947 | Susceptible |
| PI408045 | Susceptible |
| PI587829 | Susceptible |
| PI499955 | Susceptible |
| PI511813 | Susceptible |
| PI148260 | Susceptible |
| PI594541 | Susceptible |
| PI567070A | Susceptible |
| PI578332B | Susceptible |
| PI398423 | Susceptible |
| PI459025B | Susceptible |
| PI594512A | Susceptible |
| PI307882E | Susceptible |
| PI398608 | Susceptible |
| PI605839B | Susceptible |
| PI398438 | Susceptible |
| PI567521 | Susceptible |
| PI80468 | Susceptible |
| PI339863A | Susceptible |
| PI398316 | Susceptible |
| PI398962 | Susceptible |
| PI594887 | Susceptible |
| PI417130 | Susceptible |
| PI408011 | Susceptible |
| PI398918 | Susceptible |
| PI548483 | Susceptible |
| PI374176 | Susceptible |
| PI408040-1 | Susceptible |
| PI588014C | Susceptible |
| PI602593 | Susceptible |
| PI339982 | Susceptible |
| PI203400 | Susceptible |
| PI417499 | Susceptible |
| PI175175 | Susceptible |
| PI341261 | Susceptible |
| PI428692 | Susceptible |
| PI588000 | Susceptible |
| PI628824 | Susceptible |
| PI398219 | Susceptible |
| PI594885B | Susceptible |
| PI157476 | Susceptible |
| PI587627B | Susceptible |
| PI200503 | Susceptible |
| PI408340 | Susceptible |
| PI379622 | Susceptible |
| PI417206 | Susceptible |
| PI324068 | Susceptible |
| PI417208 | Susceptible |
| PI471938 | Susceptible |
| PI200546 | Susceptible |
| PI417369 | Susceptible |
| PI567025A | Susceptible |
| PI200492 | Susceptible |
| PI567095A | Susceptible |
| PI189402 | Susceptible |
| PI209333 | Susceptible |
| PI407962-2 | Susceptible |
| PI417063 | Susceptible |
| PI215755 | Susceptible |
| PI417136 | Susceptible |
| PI459025A | Susceptible |
| PI632666 | Susceptible |
| PI567020A | Susceptible |
| PI417215 | Susceptible |
| PI507301 | Susceptible |
| PI567054C | Susceptible |
| PI628825 | Susceptible |
| PI374169 | Susceptible |
| PI567129 | Susceptible |
| PI587916A | Susceptible |
| PI219789 | Susceptible |
| PI438426 | Susceptible |
| PI247679 | Susceptible |
| PI561271 | Susceptible |
| PI567399 | Susceptible |
| PI208437 | Susceptible |
| PI398828 | Susceptible |
| PI164885 | Susceptible |
| PI407790-2 | Susceptible |
| PI407990 | Susceptible |
| PI567070B | Susceptible |
| PI507006 | Susceptible |
| PI417472D | Susceptible |
| PI567088A | Susceptible |
| PI567053 | Susceptible |
| PI628886 | Susceptible |
| PI374182 | Susceptible |
| PI374183 | Susceptible |
| PI374171 | Susceptible |
| PI408049 | Susceptible |
| PI567077B | Susceptible |
| PI567073B | Susceptible |
| PI594538B | Susceptible |
| PI594591B | Susceptible |
| PI374186 | Susceptible |
| PI417061 | Susceptible |
| PI497966 | Susceptible |
| PI408003-2 | Susceptible |
| PI548359 | Susceptible |
| PI567088B | Susceptible |
| PI567136A | Susceptible |
| PI200474 | Susceptible |
| PI200487 | Susceptible |
| PI567063 | Susceptible |
| PI615510B | Susceptible |
| PI208783 | Susceptible |
| PI229358 | Susceptible |
| PI416828 | Susceptible |
| PI567039 | Susceptible |
| PI567091 | Susceptible |
| PI612611 | Susceptible |
| PI547521 | Susceptible |
| PI374166 | Susceptible |
| PI506694 | Susceptible |
| PI548402 | Susceptible |
| PI219656 | Susceptible |
| PI567068A | Susceptible |
| PI632663A | Susceptible |
| PI567270C | Susceptible |
| PI175177 | Susceptible |
| PI374158 | Susceptible |
| PI200451 | Susceptible |
| PI393546 | Susceptible |
| PI543793 | Susceptible |
| PI588023A | Susceptible |
| PI632935B | Susceptible |
| PI205899 | Susceptible |
| PI259542 | Susceptible |
| PI307853 | Susceptible |
| PI548667 | Susceptible |
| PI471904 | Susceptible |
| PI407757 | Susceptible |
| PI471940 | Susceptible |
| PI203403 | Susceptible |
| PI240665 | Susceptible |
| PI374157 | Susceptible |
| PI587880A | Susceptible |
| PI603527B | Susceptible |
| PI567089A | Susceptible |
| PI548557 | Susceptible |
| PI605869A | Susceptible |
| PI407978 | Susceptible |
| PI587867 | Susceptible |
| PI587814D | Susceptible |
| PI587560A | Susceptible |
| PI587573B | Susceptible |
| PI212604 | Susceptible |
| PI628832 | Susceptible |
| PI407930 | Susceptible |
| PI340000 | Susceptible |
| PI326578 | Susceptible |
| PI331793 | Susceptible |
| PI307597 | Susceptible |
| Pintado | Susceptible |
| Conquista | Susceptible |
| BRSGO Chapadoes | Susceptible |
| PI374165 | Susceptible |
| PI578335A | Susceptible |
| PI175198 | Susceptible |
| PI578478B | Susceptible |
| PI240664 | Susceptible |
| PI632748 | Susceptible |
| PI587950 | Susceptible |
| PI561356 | Susceptible |
| PI238109 | Susceptible |
| PI175176 | Susceptible |
| PI603608 | Susceptible |
| PI548606 | Susceptible |
| PI274453 | Susceptible |
| PI548646 | Susceptible |
| PI306704A | Susceptible |
| PI587568A | Susceptible |
| PI262180 | Susceptible |
| PI374168 | Susceptible |
| PI587709A | Susceptible |
| PI547791 | Susceptible |
| PI307889B | Susceptible |
| PI597388 | Susceptible |
| PI417009 | Susceptible |
| PI567121A | Susceptible |
| PI594538A | Susceptible |
| PI542709 | Susceptible |
| PI594698 | Susceptible |
| PI598124 | Susceptible |
| PI603605 | Susceptible |
| PI416873B | Susceptible |
| PI175181 | Susceptible |
| PI594834B | Susceptible |
| PI594668 | Susceptible |
| PI605887C | Susceptible |
| PI506500 | Susceptible |
| PI605832A | Susceptible |
| PI587992E | Susceptible |
| PI548631 | Susceptible |
| PI239236 | Susceptible |
| PI200526 | Susceptible |
| PI591511 | Susceptible |
| PI548977 | Susceptible |
| PI567056A | Susceptible |
| PI587878 | Susceptible |
| PI417445 | Susceptible |
| PI586981 | Susceptible |
| PI587984A | Susceptible |
| PI393565B | Susceptible |
| PI222550 | Susceptible |
| PI547818 | Susceptible |
| PI587828 | Susceptible |
| PI539864 | Susceptible |
| PI240671 | Susceptible |
| PI603154 | Susceptible |
| PI468967 | Susceptible |
| PI605792C | Susceptible |
| PI567378 | Susceptible |
| PI222549 | Susceptible |
| PI408330 | Susceptible |
| PI594548 | Susceptible |
| PI594667 | Susceptible |
| PI548445 | Susceptible |
| PI567230 | Susceptible |
| PI408056 | Susceptible |
| PI594707 | Susceptible |
| PI510670 | Susceptible |
| PI374178 | Susceptible |
| PI591432 | Susceptible |
| PI572240 | Susceptible |
| PI374160 | Susceptible |
| PI417120 | Susceptible |
| PI339869 | Susceptible |
| PI594480C | Susceptible |
| PI81027 | Susceptible |
| PI157492 | Susceptible |
| PI567190 | Susceptible |
| PI86876 | Highly Susceptible |
| PI88294-1 | Highly Susceptible |
| PI567078 | Highly Susceptible |
| PI560207 | Highly Susceptible |
| PI515961 | Highly Susceptible |
| PI635999 | Highly Susceptible |
| PI424405B | Highly Susceptible |
| PI2595 | Highly Susceptible |
| PI383277 | Highly Susceptible |
| PI578368 | Highly Susceptible |
| PI297542 | Highly Susceptible |
| PI407706A | Highly Susceptible |
| PI70520 | Highly Susceptible |
| PI437660 | Highly Susceptible |
| PI88826 | Highly Susceptible |
| PI84664 | Highly Susceptible |
| PI89070 | Highly Susceptible |
| PI189967 | Highly Susceptible |
| PI153234 | Highly Susceptible |
| PI257433 | Highly Susceptible |
| PI253655 | Highly Susceptible |
| PI361071C | Highly Susceptible |
| PI323556 | Highly Susceptible |
| PI398682 | Highly Susceptible |
| PI153290 | Highly Susceptible |
| PI603501 | Highly Susceptible |
| PI603497 | Highly Susceptible |
| PI295949 | Highly Susceptible |
| PI295947 | Highly Susceptible |
| PI361110 | Highly Susceptible |
| PI398313 | Highly Susceptible |
| PI189861 | Highly Susceptible |
| PI547838 | Highly Susceptible |
| PI548541 | Highly Susceptible |
| PI542043 | Highly Susceptible |
| PI548549 | Highly Susceptible |
| PI548585 | Highly Susceptible |
| PI548538 | Highly Susceptible |
| PI547589 | Highly Susceptible |
| PI595363 | Highly Susceptible |
| PI612738 | Highly Susceptible |
| PI599300 | Highly Susceptible |
| PI592524 | Highly Susceptible |
| PI562373 | Highly Susceptible |
| PI612764 | Highly Susceptible |
| PI629005 | Highly Susceptible |
| PI594822 | Highly Susceptible |
| PI417261 | Highly Susceptible |
| PI407983 | Highly Susceptible |
| PI374154 | Highly Susceptible |
| PI628847 | Highly Susceptible |
| PI561359 | Highly Susceptible |
| PI174867 | Highly Susceptible |
| PI605879 | Highly Susceptible |
| PI632665 | Highly Susceptible |
| PI632639D | Highly Susceptible |
| PI605853B | Highly Susceptible |
| PI434974 | Highly Susceptible |
| PI587871 | Highly Susceptible |
| PI434980A | Highly Susceptible |
| PI208435 | Highly Susceptible |
| PI605824A | Highly Susceptible |
| PI606389 | Highly Susceptible |
| PI548543 | Highly Susceptible |
| PI562374 | Highly Susceptible |
| PI658519 | Highly Susceptible |
| PI381657 | Highly Susceptible |

### Example 4

### Genotyping Panel

The panel chosen for this analysis was SoySNP50K (Song Q, Hyten D L, Jia G, Quigley C V, Fickus E W, Nelson R L, et al. (2013) Development and Evaluation of SoySNP50K, a High-Density Genotyping Array for Soybean. PLoS ONE 8(1): e54985. https://doi.org/10.1371/journal.pone.0054985). This panel has genotyping data for all the materials evaluated in this work. Beyond this, has a broad coverage of the soybean genome, with 42,080 SNPs distributed across the 20 soybean chromosomes.

### Example 5

### Associative Mapping of Corynespora cassiicola Resistance Loci

With the phenotypic and genotypic data from the samples used in this experiment, an associative analysis was developed in search of SNPs linked to target spot resistance. For this, linear mixed models were used (MLM) developed by the MVP packages (YIN et al., 2018) GAPIT (TANG et al., 2016) and FarmCPU (LIU et al., 2016) with the Emma matrix algorithms (MVP) and VanRaden (GAPIT and FarmCPU). In addition, a principal component analysis was performed with a value of 3. A cut-off line of 0.05 for the P value was chosen in order to determine the most significant SNPs in this analysis.

### Example 6

### Identification of SNP Linked to Resistance to Target Spot

Through associative analysis, it was possible to identify a region on chromosome 17 linked to resistance to target spot (FIG. 2). The range corresponds to 37.69-37.85 Mpb and a total of 15 SNPs significant to the bonferroni test elaborated by GAPIT were identified (Table 5).

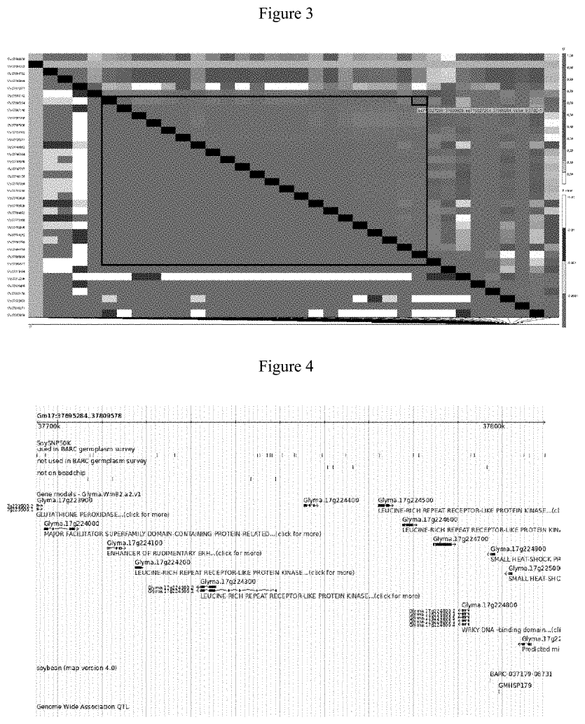

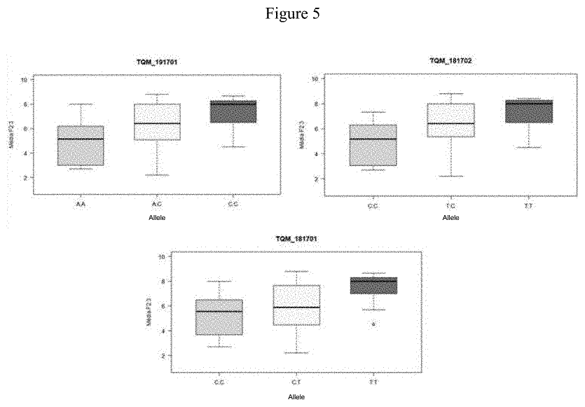

A large block in linkage disequilibrium with 110 kpb was observed, in which 14 of the 15 SNPs are found (FIG. 3). In further analysis in this region, 13 genes in this block were found to be in linkage disequilibrium (FIG. 4). Most of these genes are functionally described in the literature as auxiliaries in resistance mechanisms, but none of them have so far been associated with resistance to target spot.

TABLE 5
Most Significant SNPs Associated with Target Spot Resistance

| Marker | Chr. | Position | p-value | mAF | R2 | fdr_p-value | SEQ ID |
|---|---|---|---|---|---|---|---|
| ss715627273 | 17 | 37744962 | 6.35E−22 | 0.1799 | 0.1692 | 2.41E−17 | 49 |
| ss715627288 | 17 | 37772369 | 1.21E−21 | 0.1850 | 0.1672 | 2.41E−17 | 50 |
| ss715627282 | 17 | 37759500 | 3.14E−20 | 0.4118 | 0.1568 | 4.16E−16 | 51 |
| ss715627290 | 17 | 37781272 | 5.32E−19 | 0.4082 | 0.1479 | 5.30E−15 | 52 |
| ss715627293 | 17 | 37793768 | 6.26E−18 | 0.4133 | 0.1402 | 4.98E−14 | 53 |
| ss715627289 | 17 | 37780045 | 4.81E−16 | 0.1647 | 0.1268 | 3.19E−12 | 54 |
| ss715627296 | 17 | 37806029 | 5.28E−15 | 0.2717 | 0.1195 | 3.00E−11 | 55 |
| ss715627297 | 17 | 37809577 | 2.53E−14 | 0.1857 | 0.1148 | 1.26E−10 | 56 |
| ss715627265 | 17 | 37697148 | 1.26E−12 | 0.4075 | 0.1031 | 5.56E−09 | 57 |
| ss715627264 | 17 | 37695284 | 6.63E−12 | 0.4220 | 0.0981 | 2.64E−08 | 58 |
| ss715627310 | 17 | 37858354 | 1.14E−09 | 0.1900 | 0.0830 | 4.11E−06 | 59 |
| ss715627276 | 17 | 37747767 | 1.35E−09 | 0.4494 | 0.0825 | 4.49E−06 | 60 |
| ss715627274 | 17 | 37745344 | 1.89E−09 | 0.4465 | 0.0815 | 5.36E−06 | 61 |
| ss715627280 | 17 | 37753218 | 1.89E−09 | 0.4465 | 0.0815 | 5.36E−06 | 62 |
| ss715627279 | 17 | 37750369 | 3.43E−09 | 0.1105 | 0.0798 | 9.11E−06 | 63 |

The three most significant SNPs lie in a 27 kpb range within the identified block. In this range, three genes are present: Glyma.17G224300, Glyma.17G224400 and Glyma.17G224500. The SNP with the highest p-value is located at position 37,772,369 and is a nonsynonymous mutation under an exon of the Glyma.17G224500 gene, a protein kinase of the LRR type. The second SNP was identified at 1,868 bp downstream of the Glyma.17G224400 gene, an LTR-like gag polypeptide. Finally, the third SNP was identified at position 37,744,962 and is under an intron of the Glyma.17g224300 gene, a protein kinase of the LRR type. When using the haplotype of the three SNPs, a filtering with selection of the samples with higher resistance was observed (Table 8).

TABLE 6

| Chromo- | | | | |
|---|---|---|---|---|
| some | Home | End | Gene | Function |
| 17 | 37732104 | 37749171 | Glyma. 17g224300 | Receptor-like protein kinase with leucine- |
| | | | | rich repeats |
| 17 | 37680895 | 37686977 | Glyma. 17g223800 | Protein phosphatase 2c |
| 17 | 37691417 | 37695611 | Glyma. | Glutathione |
| | | | 17g223900 | peroxidase |
| 17 | 37696979 | 37703744 | Glyma. | Key enabler |
| | | | 17g224000 | containing |
| | | | | domain related to |
| | | | | the family protein |
| 17 | 37711176 | 37714382 | Glyma. 17g224100 | Rudimentary ERH enhancer |
| 17 | 37717367 | 37719220 | Glyma. 17g224200 | Receptor-like protein kinase with leucine- |
| | | | | rich repeats |
| 17 | 37755346 | 37757632 | Glyma. 17g224400 | AT |
| 17 | 37772129 | 37774478 | Glyma. 17g224500 | Receptor-like protein kinase with leucine- |
| | | | | rich repeats |
| 17 | 37777526 | 37779865 | Glyma. 17g224600 | Receptor-like protein kinase with leucine- |
| | | | | rich repeats |
| 17 | 37784485 | 37788635 | Glyma. 17g224700 | AT |
| 17 | 37790965 | 37792528 | Glyma. 17g224800 | DNA-binding domain WRKY |
| 17 | 37797406 | 37798357 | Glyma. 17g224900 | Family of small heat shock proteins |
| | | | | (HSP20) |
| 17 | 37801480 | 37802248 | Glyma. 17g225000 | Family of small heat shock proteins |
| | | | | (HSP20) |
| 17 | 37804429 | 37806454 | Glyma. | Predicted |
| | | | 17 g225100 | mitochondrial |
| | | | | carrier protein |
| 17 | 37810638 | 37816106 | Glyma. 17g225200 | PHD/F-box containing protein |
| 17 | 37839492 | 37840975 | Glyma. 17g225300 | AT |
| 17 | 37849485 | 37855509 | Glyma. 17g225400 | Protein Kinase Serine-Threonine |
| 17 | 37862147 | 37867711 | Glyma. 17g225500 | Spermine/spermidine synthase |

With the results obtained, it was detected that the SNP ss715627273 showed a genotype selection efficiency of 84.33%. When this SNP was compared together with other allelic variations, it was observed that there was not such a relevant increase in selection efficiency, nor in the decrease of error percentages (Table 8). This result demonstrates that the mark can be used alone to select resistant individuals.

TABLE 7
Individual selection efficiency results of the SNPs identified in this study.

| | | | Type 1 | Type |
|---|---|---|---|---|
| Marker | Position | Accuracy | Error | II error |
| ss715627273 | 37744962 | 84.33% | 61.16% | 5.99% |
| ss715627288 | 37772369 | 83.36% | 63.08% | 6.00% |
| ss715627282 | 37759500 | 83.79% | 62.20% | 5.96% |
| ss715627290 | 37781272 | 83.07% | 63.64% | 6.02% |
| ss715627293 | 37793768 | 83.26% | 63.57% | 6.03% |
| ss715627289 | 37780045 | 83.73% | 64.96% | 5.97% |
| ss715627296 | 37806029 | 75.76% | 72.87% | 6.14% |
| ss715627297 | 37809577 | 81.21% | 69.60% | 7.58% |
| ss715627265 | 37697148 | 81.61% | 67.16% | 6.76% |
| ss715627264 | 37695284 | 83.05% | 65.79% | 7.39% |
| ss715627310 | 37858354 | 79.16% | 73.28% | 8.57% |
| ss715627276 | 37747767 | 84.46% | 66.23% | 9.22% |
| ss715627274 | 37745344 | 84.22% | 67.09% | 9.22% |
| ss715627280 | 37753218 | 84.22% | 67.09% | 9.22% |
| ss715627279 | 37750369 | 84.64% | 66.22% | 9.25% |

TABLE 8
Joint analysis of the selection efficiency of the SNPs identified in this study.

| | | Type 1 | Type |
|---|---|---|---|
| Marker | Accuracy | Error | II error |
| ss715627273/ss715627293 | 84.55% | 61.06% | 6.66% |
| ss715627273/ss715627297 | 85.65% | 61.45% | 7.98% |
| ss715627273/ss715627264 | 85.65% | 61.45% | 7.98% |
| ss715627273/ss715627310 | 87.95% | 52.73% | 8.57% |
| ss715627273/ss715627293/ss715627297 | 85.80% | 60.98% | 7.97% |
| ss715627273/ss715627293/ss715627264 | 85.37% | 61.3% | 7.45% |
| ss715627273/ss715627293/ss715627310 | 87.66% | 54.7% | 8.85% |
| ss715627273/ss715627297/ss715627264 | 86.37% | 59.7% | 8.32% |
| ss715627273/ss715627297/ss715627310 | 87.80% | 54.76% | 9.47% |
| ss715627273/ss715627264/ss715627310 | 87.80% | 54.8% | 9.47% |
| ss715627293/ss715627297/ss715627264 | 86.51% | 58.7% | 8.04% |
| ss715627293/ss715627264/ss715627310 | 87.80% | 54.2% | 9.09% |
| ss715627297/ss715627264/ss715627310 | 88.38% | 50.00% | 9.53% |

A segregant population was developed by crossing BRSMG 68 (Winner) (resistant to C. cassiicola) and NA 5909 RG (susceptible to C. cassiicola). This population was advanced to the F3 generation, which a progeny test was performed on each individual inferring its F2:3. A total of 96 individuals were preliminarily evaluated phenotypically, in a greenhouse experiment, with four randomized blocks with 5 replicates per family. The same inoculation and evaluation methodology was used (scale of notes) described above (Soares et al., 2009).

The generated results were analyzed using an analysis of variance (ANOVA) and showed that there was a significant difference between the phenotyped families (Table 9). With the results obtained, an analysis of the inheritance of the trait was performed and the segregation hypotheses for the 3:1 trait were verified (a recessive gene), 13:3 (one dominant and one recessive gene) e 55:9 (two dominant and one recessive gene). To confirm the results, a larger number of families will be evaluated in future analyses.

TABLE 9
Analysis of variance (ANOVA) between individuals in the segregating
population. The data were transformed using the formula:

| FV | GL | QM | F |
|---|---|---|---|
| BLOCKS | 3 | 0.3356 | 2.50 n.m. |
| TREATMENTS | 95 | 0.6512 | 4.85** |
| Waste | 285 | 0.1341 | |
| Total | 383 | CV(%) | 12.571 |

FV: source of variation;
GL: degrees of freedom;
QM: root mean square;
F: F-test.

Finally, the three markers with the highest p-values were synthesized via Taqman technology and amplified in the 96 families of the segregating population. The results showed that all three markers had a high effect on disease resistance (FIG. 5). The presence of the susceptible allele in all three markers was associated with high disease severity values in the segregating population, this demonstrates its high efficiency in eliminating materials susceptible to the disease. In this way, the high applicability of the tool for discarding genotypes that do not possess the disease resistance gene is demonstrated.

TABLE 10
Sequence of the markers most associated with target spot resistance observed
in this study.

| Marker | Sequence |
|---|---|
| ss715627273 | GAAGTTAGATCTAGTTGGCCTCTCATTGGTGTTATGCCCGAAGAATTGCTGCA |
| | AGACTCA[T/C]AGAAGGTATCTGGGGTACGCTAAAAGGAAAGTGATACATCG |
| | CATGTGCCTCTACAATGA |
| ss715627288 | ATTGTTCTCTCAACTCAGACATCGGCAATGGAGTTGGACGAGCCACCTATGGCCA |
| | ACCTCTCTG[T/C]GCTTCAAAAACTCTTCTAACGGATGTCACAGATTTTTCTAC |
| | TCGCTTTTCGTTCACCA |
| ss715627282 | AATCCTCCCAAGAATTCATACAATGTGTAATGAATCAAACTAAAAGCCTAG |
| | AAATGAT[A/C]TACTCTCTCACAGAACAACTGCTTCAATTCGTCCACTGATGAC |
| | TCTTCATTTGCACTCTA |
| ss715627290 | AAGAGAGTTCTAACCAACATCCACGTCGTTCCTTCACCATTGAAAGAGAGCTG |
| | CAACAGA[A/C]AACATAGGTGACGGTTTCACCTTTTACATAGGCTACCAGATT |
| | CCTCCAAATGCAACTAAT |
| ss715627293 | TGAATATAATGTGTAAAATGCATTGAAATGAGACAAAATGAAACGAAGT |
| | GTAATGGA[A/G]GTAACTGATAAAGCAAAAGAGAAAGAAAAATATATATAT |
| | TTTTCATTATATTGTTATGA |
| ss715627289 | AAAAATAATTAAAAATTCGTGTTAATCAATTTTACAAAAATCAATGT |
| | TAAAAAA[T/C]TCGTGTTATTTATGAAATTGTCATCACATTTTTATTAATCTA |
| | TTTTATGAAATTAAAA |
| ss715627296 | GCGGGGATTGTTCCCATTAAGGAAGTGCCGAAACCTCGGTAGAAACCTCGGA |
| | AACCCTCG[T/C]AGCGAATGATGGCGCGTGACATGTTGCGGCACGAGATTTTG |
| | GCGGAGGAAACTTGCTGAC |
| ss715627297 | CATGGTTAACGTGTGATCGATGAACTCTTCTAGAATGTATTCGAAAGATGGGA |
| | ATTGAAA[T/C]TATAATTTTAATTAAGCCTTTTTTTAGAAGTTAATATAAAATGTA |
| | TATTTTAATATTTGAGT |
| ss715627265 | CCTTCCCCATGAAACAGAGCCAATGGGTGAGAACGATAACAAAACCAAAA |
| | AACCTCCT[C/T]TCCCTCCTAACAGAGCCAATCCAATGGATCCAAAGTCTCTCC |
| | TCACACAGCTCTCAACCCAACC |
| ss715627264 | GCCTCTGATTTATTTTGTCAGAAGGATCCAGAAGTTACTTGCTGCCTGAGT |
| | GTAATTC[A/G]GAACACAAACGAGAGCTGTATGTAAGAGCACGAACCGAGTG |
| | ATGTGTGCACAATAAGTTA |
| ss715627310 | TTATGGAAAAGAAGGTAAATGAGGGGGCCACTTGTCATTAAACTCTACTA |
| | CCCCCCCCCC[T/C]CCCCCCCAACTTGGGAGTTGATAAAAGGTAAAATTGTAAAT |
| | GACGATTCCAAACATAGCC |
| ss715627276 | AACTTATTTTTATAACTTTTGCGAGGAAACTCCAATTTAAATGAAATTTAA |
| | GGATAA[C/T]CGTATGTTTTAACACCCAAAGAAGAACTCATTTTTGGCATAAAAAAC |
| | TCAAGGAAAACATCTC |
| ss715627274 | CGTAACTATCACACTTATTTCACAATAGGGCCTAATCACTGCCACCAATCCTC |
| | CCAGTGT[A/G]TCTCTATCCATCATCATCCACGTCCTTAATGTTGGATCAAGTGGTC |
| | TCGGAATAATTAAGAAA |
| ss715627280 | CCTTCTCCTTACCAAATACCTTTTTTAAAGATAGCTAGCCTAGAACGTCTTA |
| | CGTCCT[A/G]GTGTTGAACCTTTCCTGGTCCCCGAATCTTGATCTATAAG |
| | AAGCATTAGATGCACT |
| ss715627279 | TCACACATTCTCTGTTGAAACACTACCAAGCAAGTCAGACCCGACATGGAGTGC |
| | GTGTAACG[T/C]TGGGGGATTATTTATTGAGAATGTTACCATTTTTAGAAAAG |
| | ATTTTTTTTTTTTATAGTAA |

1. A method of identifying, distinguishing and selecting plants of the genus Glycine, resistant or susceptible, to target spot caused by the fungus Corynespora cassiicola, the method comprising:
(a) Extraction of nucleic acid from a plant of the genus Glycine;
(b) Analysis of extracted nucleic acid for the presence of one or more alleles of the molecular markers associated with increased resistance or susceptibility to Corynespora cassiicola within a range of 37.69-37.85 Mpb of chromosome 17;
(c) Selection of the plants that possess the mentioned alleles of the markers.

2. The method according to claim 1, where one or more markers are located in the genomic region of the genes or in the ranges of the genes Glyma.17g224300 (SEQ ID NO: 1), Glyma.17g223800 (SEQ ID NO: 2), Glyma.17g223900 (SEQ ID NO: 3), Glyma.17g224000 (SEQ ID NO: 4), Glyma.17g224100 (SEQ ID NO: 5), Glyma.17g224200 (SEQ ID NO: 6), Glyma.17g224500 (SEQ ID NO: 8), Glyma.17g224600 (SEQ ID NO: 9), Glyma.17g224700 (SEQ ID NO: 10), Glyma.17g224800 (SEQ ID NO: 11), Glyma.17g224900 (SEQ ID NO: 12), Glyma.17g225000 (SEQ ID NO: 13), Glyma.17g225100 (SEQ ID NO: 14), Glyma.17g225200 (SEQ ID NO: 15), Glyma.17g225300 (SEQ ID NO: 16), Glyma.17g225400 (SEQ ID NO: 17), Glyma.17g225500 (SEQ ID NO: 18).

3. The method according to claim 2, where the markers are located in the genomic region of genes or in the ranges of genes selected from the group consisting of Glyma.17G224300 (SEQ ID NO: 1), Glyma.17G224400 (SEQ ID NO: 7) and Glyma.17G224500 (SEQ ID NO: 8).

4. The method according to claim 1, where said marker is a SNP selected from the group consisting of ss715627273 (SEQ ID NO: 19), ss715627288 (SEQ ID NO: 20), ss715627282 (SEQ ID NO: 21), ss715627290 (SEQ ID NO: 22), ss715627293 (SEQ ID NO: 23), ss715627289 (SEQ ID NO: 24, ss715627296 (SEQ ID NO: 25), ss715627297 (SEQ ID NO: 26), ss715627265 (SEQ ID NO: 27), ss715627264 (SEQ ID NO: 28), ss715627310 (SEQ ID NO: 29), ss715627276 (SEQ ID NO: 30), ss715627274 (SEQ ID NO: 31), ss715627280 (SEQ ID NO: 32) and ss715627279 (SEQ ID NO: 33), or combinations thereof, or any other molecular marker in a range up to 5 cM or 1 Mbp from said group.

5. The method according to claim 4, where said marker is a SNP selected from the group consisting of ss715627288 (SEQ ID NO: 20), ss715627273 (SEQ ID NO: 19) and ss715627282 (SEQ ID NO: 21) or combinations thereof or any other molecular marker in a range of up to 5 cM or 1 Mbp from said group.

6. The method according to claim 1, where the identification of the markers is by any amplification methodologies, or by use of probes, or by any type of sequencing (e.g. tGBS or directed sequencing).

7. The method according to claim 1, where the plant of the genus Glycine is Glycine max.

8. A method of introgression into plants of the genus Glycine of alleles of resistance to target spot caused by the fungus Corynespora cassiicola, the method comprising:
(a) Crossing parents of plants of the genus Glycine identified by the method as defined in claim 1 with other parents lacking said resistance;
(b) Select progenies possessing markers associated with increased resistance or reduced susceptibility to Corynespora cassiicola by the method as defined in claim 1; and,
(c) Backcross in one or more cycles the selected progenies with the recurrent genitor to develop new progenies.

9. A nucleic acid molecule, characterized by being able to hybridize with any of the SEQ ID NO: 19, 20, 21, 22, 23, 24, 25, 26, 27, 28, 29, 30, 31, 32, or 33, or subsequences thereof having at least 15 consecutive nucleotides, or sequences with at least 90% sequence identity.

10. A method of using a nucleic acid molecule characterized by being able to hybridize with any of the SEQ ID NO: 19, 20, 21, 22, 23, 24, 25, 26, 27, 28, 29, 30, 31, 32, or 33, or subsequences thereof having at least 15 consecutive nucleotides, or sequences with at least 90% sequence identity in the method as defined claim 1.

11. A detection kit, comprising at least two nucleic acid molecules as defined in claim 9.

12. A method for genotyping target Glycine plants resistant to target spot, the method comprising analyzing the presence in the DNA of the target plant for one or more markers associated with target spot resistance, selected from the group consisting of ss715627273 (SEQ ID NO: 19), ss715627288 (SEQ ID NO: 20), ss715627282 (SEQ ID NO: 21), ss715627290 (SEQ ID NO: 22), ss715627293 (SEQ ID NO: 23), ss715627289 (SEQ ID NO: 24), ss715627296 (SEQ ID NO: 25), ss715627297 (SEQ ID NO: 26), ss715627265 (SEQ ID NO: 27), ss715627264 (SEQ ID NO: 28), ss715627310 (SEQ ID NO: 29), ss715627276 (SEQ ID NO: 30), ss715627274 (SEQ ID NO: 31), ss715627280 (SEQ ID NO: 32) and ss715627279 (SEQ ID NO: 33), or combinations thereof.

13. A Glycine plant resistant to target spot, where it is obtained by a method as defined in claim 7.


SEQUENCE LISTING (33 sequences)

SEQ ID NO: 1 - DNA, 17068 bp, Glycine max
atgagacttgaggagaataaggttgatcaatgcatatacctcaaggtcagtgggagcaaatttatcttccttctgctatatgtggatgatatattattggcttttaataaccttggcatgttgcatgagacaaaggatatggcaacaagatcttttgacatgaaggatcttggtaaggactcttttgtgttgaggattgagattcacaaagatagatcatgccatacactcaagttatcttaaaaggcttacattgatagagccttagagggattcaatatgtagaattgtatacctggagatgttccaattgtcaaaggtgaaaaattcaacaaggaccaatgtcttaagaatgagattgagcaagtagagatgaacgacatacccttttgggagtgcttgtaacaccttaaaattttcttccaatttatttttctacacgcataaaattgcactgttaaggactagacaatgaaaattttgcactgtgtaattttattatatgcctgtgtgatttaatttattctttgtataagtttagtgttagttatattgatttttttgtgtaaataattatttagttagttggtagaaatttggaaattgactgaacaaaaattccatctatagcaaaattgcaccatgtattaagtggtagtccaatttatctttgtgtgaggttcaccctgggtacttgtattcctagggagattagcacaacaacataggcgattaagataagatttactaattttggtataaaaaacaaaaatacccatttttggcaaatcctatcccttccatttttggttgttttgcaaaaagcaaaactccccatttgtggcatgaagtggcacgttcaaaaaggtcagccaaggggtcattcaagtgtcaataagccttgtcaccttgaagcacatgccttatctccttgtccattgtggaaaaaaaattgaacccttcattccttcatcttcatgagttcaacattgagggaaggaggtccaacttttctttttcttcttcttcctagctcgttcctagtgttcttgagccattggtccatcaagcctaggtaaataagtttcattcttcatcctagacttttctcttcgttctcttgttctagcttctctaatgacttaaaattgtcattttgttttacttatgcagggaaacctttggaaagcttaacattcttcattctcccttcaaaatttcgtgagtttcatcaagaggtaagggggggtctcttgaactcttgaaccctatgcttgttgacaaacttccttgaacttgttttgaaatttttgagcttgctgccatgttctggatttgtgtgctgagatgttttccttgagttttttatgccaaaaatgagttctttgggtgttaaaacatacggttatccttaaatttcatttaaattggagtttcctcgcaaaagttataaataaaataagtttatggaccttttgtaaaatgaaactttgtcacagtagtacggactgtttttattaaagttttgacctcaaaaatgagtttttttaggtttgaaaatgtagggtagcatataagatttgcgaaaatccaattctcagagcaccgagagcaccaaaaataagctaggaatggaactttgtaaaaccaagcgacaagatgattttagacggtagatagcttaactttggaatctatgcctccatataaattaccttagaattattattttttttggaactagaaatttatttgctattatattgctgagataactacaagatcttatatctgatgcatgttctaaaattatgatgattctttgctgataaaattactgaaatttgtgcctatagtttattattaatattgaatgataagaccgacagagtgtgaactctgaaaattatatttgtggcatgaaaaattattttctattatatatatatgcatattgtcattttatctgcacacattgtggtttgggttcaaaggtgttcgaccctcaataaaattatttgttatgtatggttagatagctataattacagatgttgtgttgtctaattattctgtgacaagtggttcaaccgtataaggggtcacgattgattccctaagagtagtacgtaggcttgagccttgtctctttttcttgtttgtttgtattcgttcgttggatatagattaaggaactagtggttctattttcttctatgagaaggtgaggttacgttaagtgtagaagaaagcttcatgatgatgaatcaagttgactcaagatgttttgatgataacaagagatgatgacaaaaagcccaatagaatgatttcaagattgagtcaacaattcaagaatcaagagaagtttgatttcaagattcaagaaaagataaattcaagtttcaagagaagaaatcaagaagacttcacaagggaagtattgaaaagttttttcaaaaaacaaacatagcacaattttgcttttcaaaagaagttttcacaaaattttctaagttaccagagtttttactctctggtaatcgattaccagtttcctgtaatcgattaccagtggcaaagtttgttttcaaaagctttcaactgaatttacaacgttccaattaatttcaaaatagtgtaatcgattacaagatattggtaatcgattaccagtgtgtttgaacattgaaattcaaattcaattgtgaagagtcacatcctttcataaaatgctttgtgtaatcgattacaaggatttggtaattgattaccagtgacaagttttgaacaaaaatcaaaagatgtaactcttcaaatggttttcagtttttttctaaaggttataactcttctaatggttttcttgaccagacttgaagagtctataaaagcaagactttgactttcatttcaataactttttcgaattgacttttgaatatcttcttcttcttcttcttcctttgaaaaaagctttcaaaagttttctggttttccaaactttgaaaacaagagtgtgttattcatctttttcattctcttctctctttgccaaaaagaattcgccaaggactaaccgtctgaattctttatgtgtctctcttctcccttttccaaaagaacggaggactaaccgcttgaattcttttgtgtctcccttctcccttgtcaaagaattcaaaatgacacagtctgagaattcttttgattcttctctttcccttatacaaaagatttcaaaggactaaccgcctgagatatcttttgatccccttcacaaagttttgaaggactaaccgcctgagaactttgtcttaacacattggatggtacatcctttgtggtacaagtagagggtacatctacttgggttgttgtaactaagaacaagaaagggtacatctcttgtggatcagttcaagtggagggtgcatccacttggttgttcaaagagaacaagggagggtacatcccttgtggatctttgcttgtaaaggattttacaaggttgaaaagaaatctgaaggaccgcaggtcgcttggggactggatgtaggcagggttgttgccgaaccaatataaaactcttgtgtttgtcttcttcttccctacactctttaatttccgttgtgcattttaattatcgcttttacttttggttaagtttttatttctattctttattttcttaacaacatagtaaaagcctaagaagggtagatttttaattagtaaaggttcagtaataattaattcaacccccctttcttaattattccgagaccacttgatccaacattaaggacgtggatgatggatagagatacactgggaggattggtggcagtgattaggccctattgtgaaataagtgtgatagttacgcgctaaggatgcgacagtgttgtttgtttagataaccacacatggtagttacgtagattgtatgctttagttttgttttgctattgcctgttgtttgtgatgttttaggagcggaggtcattgagcatccatggaggatgttcacttggatcaatagctaccgctatagctaaacgatggaggaagaagtgcacctccacacacactttggttacatccagcaccactttttgctgttagatgagccccagataccactgatcattgtagaggcacatgcgatgtatcactttcctttttagcgtaccccagataccttctatgagtcttgcagcaattcttcgggcataacaccaatgagaggccaactagatctaacttctctcaggttacgaccatcgtggtctacgaggatgtgggagatttcagcgacaatgaggatctcatgccatctgagatcactttctaccgagatgtctgcggtgaagagatatttgagggaaaaatggtttcgtctgacgaggtacgggacttctcgagagacaagagtgacctttatgaggatatgacttcaccctaggtagcttgtgggtcttggatatcgactgctctgagtataggattttttccctttttgatgtatctttagagtggaaagatttctataatcagtgtcatgatattttattgtattaaacattttggtgattctttgtgatatttggaggggcaatggatattttattgtattaaacaattgtaatgattttagtacacttatgtttaaatcttaagggtcactgaggctcaataagtatgtttacgtatttcttccaatttatcatcatttaattttgtgcaatttatttctttataatatgtactttattataacattgtgtaaggacaaaagaaaaatgacaacaactttcaaaatgtttttttaataaagaatgcattttgacaatacattttattcatgatcattttaattatacgtacacgttttaatcaagcgcaatatatttaattatatgtgttccttcttttctcaaaagaaaagaaaatgtattgagatattttatcaacttagaaagaaaatctaatttaatgatgcgtatttcgcaaaatatattatttaaatgtttcaaagtaatatttcaaaaaaataatttaaggtgttatagtgctctgggctgcctcatgtatgctcatgatcaggtatgcactagactgaataatgctttcatagttggcgttcttggtacataccaatcgaacctatgaatgatcactggatagttgctaagaaggtgatgaggtacttgtagaagactaaggagtatatgcttgtgtataggagagttgacaacttggaggttgtgaggaatcttatgacaggacttcgcattgtggactttattgctagcccactgaaaattttttgtgataactatgttgctccattttatacaaagtgtaataagacttctagaggatctaagcacttggagttgaagtatctgactgttagagatcttgtgaaagatggttcgatagtggtagatcatgtggacacaaattcaatgttggttgatcccttgaccaagggattaaggcttgttgttttcaaatgacatgttgaaaacaagggcattgtgaatgtttttgatatttttggttagtgggtgggggctttagatttatgttatttcctttttttcatcttgttggatccttattttgaattttgaaaaatgatgtattgatgacattttacatttgatttgatgaataaatgattatatgaatttcattatgataacttttgtatgtgtatatcaaatgtaagattattaaagctcagtgtcgttaggaccattatgctggtggtcatcgacaccagaactgaagctgaggcataatgatattaaagctcgacgtagttaggatcactatgtcagtgatcgacaccaggattgaagctgaggtctgatgatttcttgatagaacatgtaatttctctggctttcaagcacttgagagaaattaaggactaacgaagaaaatgacagtatggctttcttaatcacattggcacatcattttttattacactctacattgatgactagtcgacgaagtcggtaatatttgtaaccatggaaagtgttgcatcaatgagagatgtcattatcgccataattcagtgttactggcttttgtagcatgaagtcttaattaagtgttgtatcttgagatcatttgttttgtggttacgatatgtcattagtttgagagtcattttcgaaataatttttaaaattaaaaatgcacaattaattttgcccaagtggaagaatcttaaaatttttccatatattcttgtgggctacaattaatagtaatattttgttttgccaaaagcaggctactattatgatccatttgatgatacttaaatccactaataatgtgattaattgtttacgagttttgtccacctaattctaattagtatgggaaaacagtgtgagcctaaccaccggacccataatgggaggtgcttagggttaataaatagcaaagctaggtcccttttgctgtcacattatgaattgctctataggatttgttccattgaaaactctacaataagaagagtctgatcgggcaaaggaagacttgacttttggatttctacaggtcacactatacatagacaattctcatcttcttgatcatgttttcaacaaacaagtgttatgtccatattttaaactcatgaattcgttcttgataaatttatttattttacatcaatctcattacattaagctgctgctgtaagactatttatactttatcggcttgatccaccgtgattgacaattacaaaatatcagtacatgtcacggtttctagttttttcataagaatatggctatacttctctatagtacatttacatctagattaatctaggactttcttttccttctctagaatttgcatacccgagtaataccagaaaaggatcgaaaatataaaggccataatgacaaaagatcatacagcagcctaagcaatcataacatatcccacaaatcttaaaaaaaggtaccaaatttcagaaagctaactaagtgggatgataagtctatttggaatagcagtgtgtagttgatccagaatccttttcaagatgttctttgtgttaattgttatggctccatttggccaatcacctttggtgaattattttctacatcggactagtatatacttagtggaataacttgtcaattgtcattcttggctcgttcatctttaatcaagggagcttatttttctaaatggatagtacgtagtggaaaaaggtatcaaatttccaaaagctagctaagtaggatgatacgtccaatggagtatatatagcagcgtgcagttgatccataatccatgtcatgatgttccttatcttaaggttccattttgccaatcatctttagtcaaggaagcttattttctacatctgaacgaatgtatgtatgtacgtagtgggaataacttgtcgatcaattgtcattcttggctcattcacttttgatcaagggagcttatttttccacattagaataatatgcagtggaaaaaggtaccaaatttcagaaagctaattaactaagtgggatgatatataagtccaagggagcttatttatttatatctgaatagtacattacatagtggaatcacttgtcattcttaataatagactattttttgtaggcaaataaatttattaattcaaaacaatgttgatggagcataagaaatgctccaaacaggcttgcatacagatgatacatcagctaagaagatacaatacatattcgtatctaacataaaggaaccatattctatggatactaacataatccctatcttcttcgttttatcagtaaatactagtttgttagaagcgtcagtgggggattcgatcccgcaacctcttccacctcaacttctcccttcacccttaagtatctccaatcctaactactaaaccaaccttatatctccacataattcctatcttaagcacttattatttgccatttacattattcacccgagcataagatagcaacttaattaaccctgcagccacatcatttgttatgctatcagtacgacgcaccttgttattgatagacctttaaccatgtgccaaaactgatatgctcgtactcctcccatccaccaatgttgttcaggcaaagcagaggagtactattggtccattcctaaaataagaaggtgaatcctttgatttttacttgtaaccaagaccatgttctgagtctaatgctgtccaatagtgcttctctgtccaaattcacattcctgaagactactacatcatggcggtgtctccacaagcaccgcaaaattgctattcgagtcactttctacgcttgagaaaccttccaaaacccaaggaaaagtcacaagactcatgctctactaatgcttgcagtactagcaaatctctctgtatactgacaagaatccccccctccagcaccacgtatattgagttcttgttgtgaagatttagtttcaattataaatgtgtgaataatcctatatcataccatcgaaaccaagtacttatgtgataaaacaagcaccgcagagaagttttctagtcttctcaatatgtgacatcaaacaaatattcagattactgaacatgtatatttacttttggattcatagtatattatttcatttttttatttactaatgcacaataattttattattttatattgacgcagtcactctttatatcatataggaatcaaaacttacactaaaaatatattaataccacactataatattagactatttacatgaaaacatatatgatacataaatcacatacagaactcgtccaaaattggctaaatatatgttctgatatattgcaaggccttgaaataggcatgctgttggtgatggatggtgattcaaaggaatcagattggccattggtaggctgaggaggacaacgagggcatgtcacatagaaagcaatggcaatggcgtttcttgcttcagggcattaataacctgttctgcctttggccttttcttgtgatctgggtggctacaccatattcctaccgtgagcaaacatgtcatttcgttcacatcgtaatcccctttcaatcccttatcggcagcattcaaaatattcccttccacataatgtttccatacccaatttgttaaaggcacatgattatgctccctatcctcataagtcctctttccacatgctatttccaaagccaaaaccccaaaaccgtacatgtcagattccttgctagcccttccttcctttacatattcaggagccaggtacccatatgtccccaccacctttgtcttctgagtcctcaatcttggatccaccaatttcgccatcccaaagtcactaaccttggtattgaaatcagtgtccaacaaaacattagccgacttaatatccctatgaagaacacattgttccgcgtcttcatgaagataacgaagtgctctcaccacgcctaatgctatcttgtatctcacaccccatgtcaaagttcttctgttgccaaagagatgattgtcaaggcttccattagtcatgtactcaaaaaccattaataacttcccttcctcgtggcaccaccccatgaattgaacaagatttttatgtatgagacgacttataatcttcacctcgttcgtaaatatttcttcagaattttcaacatcagaaaaaatccttttcaccgcaaccacgcgccctaaatcactcagaaaccctttgtaaacttctccataacctccttctccgagccttctatcatctgcgaacccgttggtggctgcaactaattcgttgtaaccaaaccttcttggcatagatgctttatccaaatcagaactatcttctcttcttgtcttcttgatgaagatcaacaaagaaacaccaatgatcaaaagcaccaaaaatatagaacaagtcactgcaacaactactagtttgacatggaacttgtatttaactatcatgtcagtgttttctttgtttacctctaaactggtggagttcagagttgacgcaaattcccaggaatgaataacattttgctcactagacgacccggttgcacctgaaaacccaacattgacccattctggtaaagtttcccctaggtcaatatggtacgaaagagaattagcattaggtgtaaaaccagagctagttccctcaaagaaccacgagacagaaaggagtttggcagaagcattgtgagttatcaacgcatgccccattttccctatgttgctctcaatatcaaacctgctataattgagggatgccacagagttattgttgatccctacgtgctgcactggtggatcatcatcgtttacataagtgtcgaattcgacagcaacgatgttgttttgggtgtcaccgtataggccgagagtgctaccatctgattctgatttttgcggaatctggaagagaaggggtgccacatagaaggcaaagccgtcaccatagttggtttgagtgggcacgccaatagtgaacgaaaagcgagtagaaaagtctgtgacatttccattagaagagtttttaaggtgcaaaggttggccatagatggctcgtccaactccattttcgatgagtggattgagcactatggttccgttctcaatcttggctacgcctgccgttccaatgaggtttgcgctttcagtgtcattgaagttaggtatgttaaaggagagtggttgaacaattggaaggataataaaaaggaagcagaaaagaagagaagttttgaagtagtactggaaatgttccgaggtagccaacatggttcgttacggctgagtctgctaggcctgagcctgaggatgttaatttggaattgtcttatagtatatttgtagggaagacatatggaacacggcaatttatatcacactacagggtagattatataaaaaaaagatgatttatagacccctaaccaaatgagagttcatgagtcataagaatttttttaattttaaaactgagaccttccacgaaaaagtttacagagatataaatttaaagctaattagttaggcaatgacttttataaaatgctagtcggcctaggatgttaaaattttttttatcatgattaacatacatacatacaagcccatttaattaagtacgaattggacatgaactcttgataggagttaacgacgatgcaagtgtaattaaagcatatcagtggtaattgctaagctttaggatcatcattgtatatagttgtagatgcatggacttgcgcttaaggagataatgatatcatttagatattaaaaactcacactagggatagattattatagtgtaagtgtgtataaaaggtctcacaccagatataaatgaatgttgataaacatgagtgataataatccgtaaatctaatttcttaatattttgggttagatgttgttaactaggttcacttgtgtgcttattaacaagtggcccatcaataatgaagatgcatgtccttaatgttttatcccccaactcttaacaaatagtatgtagaggtttcatgttgaatgtgatgtcaaatttacttgtatatttgactcgtgacccattaataaagatatctttagtttttacacctaattcctaataaagtggaagtcataatgcaacttgatgatgggctcaagcgagtattaagtcatggcataatcgatggaggatccatgtataggaattccttgtgatgatgaaaatctttatgacacttgtgtagataggccaaatagtgtgtatataattgatgtgagttaataatgatggaagtatagatgacaatagttaagcactaaagttatcgttgaatattcttgaatgataagacttccgcttgatgagggaagcataccataccaaaaaaagtctcacacttaaaggatagatcaaacacataaaaaagtcattttaacctaagaacataaaaatatataatatcacacgaataacacatcaaaagctaattaaaacaagcacaagttattaatttattccttgggtaatcatcaaaaaatcctttgacctatagtttagttaagtattatcagcttaaatgaatttttcgacctccaattatcacatttgtgaaattttggtctctaaattttaattatgtgacttttgtattctaaagtattagaattgtgcaattttagctcttcttcaatcttcttttaaaaatttaatgatttttgttaagatgacattgacgaatgatatagaagaggatgtggtattgataaatgaaattggcgtgaggacatgagctaatgtgacagatctcaatgtatcactaacacggcagagttatgggggacatgtaaagattgggggcgtgtgaaaataagaatgtaaatcatcaaaatgttaaagatatgattagaactccaaacattaaactctaagaacaataattaaccattagactaacaatttttttatatacttcttttttttttattgattgaaatttattgaaaattattaatacatgtcatttaatgattctcttttaatgatttgtaggactcattaaaatttaaaatttataacataataaaaactttcaactaaaatcaaatttttaaacttttagttaatatttatcagttgtgtttactaagtatacactaacaaagactaaaagatagtcaatgtaattagctagctgtactaaaatatctatgttctagatggaaaaaaatcttcttacaaataatttctatgttgaatgcttagcaaacattaaaatacaaatgatttatcaaataaaccttgccgtggtggagtggtggtccaattaattaccaatttttgtcaaaggagtttgttttactcgcttgaattgtagtggaataattgcaccccttcttggctttttctactgaccctatgcatagaatacttggctcaattagttgacttatcccattgtgaagcttcttcttaattacgttggccagacatcatttaatgatttaataacgtttgaagctaataaaagttgaaaaaagagaaagaaaaaatgtcctgcacacacgacatattcacgttactgtcatgaaatttaggtatctgccactgttattacgtcaattagtaatggtggccctgcatttagcactaccacaagaccaattcaaaataattacagatgtcagttagtgaaagtgagtccacaatgtaacatagtatttcttctgatctcaaagatatcaaatacaagaagaagaaaaaatatattatattaattaagaaaattaattaattacaatttgaattgaaaaatattttttaatttatttttttattagaatttgatataaaaaataaaaaagaattattgaatatagaagattaattaaataaaaaaatattttagaaataataacattaaataagataaaattaataaaaaatttcttatatttaaaataaaatattcttttatatttgaaaacaaaaatacacaccaccaagaccaattcaaaataaaatagtgccattgactaccaagaccaattcaaaataaaatagtgccattgactcagtacagttagtaaaacttcacttagttttttttatttaaacagcaagttccatcgagcaataactcagaacaactaaacttaattaatcactcctcgaaacaaatctacttttgctattccacttctctcatcacccctttcatctttgatatgaatttcttagctctatcaaacaaaggaagaaaaaaacagccacaactgagattactggagatggtgatcacagtttttctcctcgtgttagccattccttcacctcttaaaacagcagaatcactaaatttcaacataacaaacttcgcaaactccgagagtgcaaaaaacatgttatacgtgggtgacggcgctgtgaacaagaacgggtccatagagctcaacatagtggactatgatttccgcgttgggagagccttgtacggacaacctctgcgcctttgggactcatcttctggtgttgtaaccgacttctccacgcgcttcactttcactattgacagaggaaacaacaaaagtgcttcttatgctgacggtttcgcgttctacatagcccctcatggctaccagattcccccaaacgcagctggtggcacttttgcactcttcaacgtcacctccaatcccttcattccccgtaaccatgtgctcgcggttgagtttgacacatttaatggcacaattgaccctccctttcagcacgttggcatagacgataactctctcaaatccgtcgctaccgccaagtttgacatcgacaaaaacctcggaaagaaatgtaacgctttggtaaactataatgcttccaacaggaccctctttgtgtcttggtcttttaacggagccgcaacacccaattcgaagaattcatcagtttcttaccagattgatgacctcatggacattctacccgagtgggtggatgttgggttctccgcttcaactggcgatctcactgaacgaaacattattcattcgtgggagttcagttcaacgttgaattcttccactgtttcgaataacaattcctcggacaataatggagcgaaggaccggaacgggttgagctcggttgcggtggttgctgtggcggcgtgtgcgattgttttggtggcagcagccgcgaactttgcagcttgggtgataatcatgaagaagagaaggggtaagggtgattattatgataatgatgaaagtggacacacatcggctaagtttgatttggatagggaaactatacctaggagatttgattacaaagaactagttgtagctaccaatgggttcgccgatgatacaaggcttggacgaggaggttcgggacaagtttacaaaggggttctgagtcatttaggaagggtggttgcagtgaaaaggattttcactaactctgaaaattcagaaagagttttcatcaatgaggttaggatcataagccgtcttatacacagaaaccttgtgcagttcgtagggtggtgccacgagcaaggagagtttctgttggtttttgagttcatgcctaatggaagcctcgacagtcatctctttggggacaaaaaaactttgccctgggatgtcaggtatggttacataatatatataatcaattcaattatatgttgttttatgtatttttataatgaacaaaacttagttgtttcatacttgtcttagtagtctatgttaattaatctttaataatacagacaaggaccgatatagttagggaattgggggaggggtctttttaaaagttgaaaaataattttaaaggagttatttttaatctttatcctttattttctaatctaattggtcaaaatgagtaactcccacttttaacaatttgattaaatgacaatgaagagtgtagatagagagtaattattcctatatacacaatttagttgtagaagtaatagtaattaagtgtgtttatttcttttttactttttaagaacatatttaatttctatttttagtatttattgtaaaaataataacaacatttatttcttttgttaaataaatatactatttcatttttatagtctttattgtggagataattaattatgttattttttcactttttaaacaaatatattcaagaattatttatttctctttttaataatattttcaatttataatcacctattgttatttcaaagtttgtatgtaaccttgaaaaatactataaagagaaactaaatattgaatacaatttattccatggacacaaaacaaaatacgcgcgcgcacacattattagttaacatttatattaaaattgacccgtcctatagtggggtatgattccatcactgaatctgaataaaaacttgtattgcgttggttatatcacttatggtacttcaattatgaagagagaacaacactaataaatacctatgaatatatgcatctatattttgtgtttttattttttattcttgtttaaaggaagtataatcttgcattataatatatataaccttgcaatcagtaccctgtgtgtgttatgcaattaaaaccttttccactattgttctcaatttttcacacaggtacaaggtagcattaggtgtggctttggcaattcgttatcttcatgaggatgcagagcaaagtgttctacatagggatattaagtcggctaatgtgttattggacacagattttagcacaaagcttggtgattttgggatggcaaagttgttggatccaaggttgaggactcagaggacaggggtagtggggacttatgggtaccttgccccagaatatatcaacggaggtagggctagcaaggaatcagacatttatagtttcggggttgtggctcttgaaattgcatgtggaaggaggacttacaaagacggggagtttcttgtgcctctcgtgaactggatgtggaaattgtacgtggaagggaaagttctggatgctgttgatgagagattaaacaaggagtttgatgttgatgaaatgacaagcttgattgttgtagggttgtggtgtactaaccctaacaacaaggaaaggcccaccgctacacaagtaataaaagttcttcagcttgaagcgccattgcccacgcttccacttgatatgcatgatggtcctcctctttctttaaacacgtatacgcatgcacaacccccttatgattccttgcaatcagtacccttcactaacagccttcaaagtgttggacgctaagttgctaagtcaagcttcaaatttatctatttcgggcactatgaagttgtgcaatctatgtagaatttcattttttttttatgattttaatttgatgacctttgtgacaaaatgtttggtgacttaacagatatgtaaaatggttggaagagagtttccttttgaataaataataatgcttatttcacatcttacaattttcacatatatt

SEQ ID NO: 2 - DNA, 5818 bp, glycine max
tactcttcatctttctcactattcgtttgttcatacttctcagttatcaatagtttaaaaaaaaagaagaagaaaaagttaaaaaagttttcctcatgaattgttgcttctcatatagttttttcttttcccactttcaagttagaatacacagaattatgagctgtgaactcttattattcatttaatgttcatatgatcatctgggtctttcttgattatggagtctactaccttctgtcggtctgttgattaatcttgatgtttattatttattttctatacaattcttagcacaattcaagacataacgaatttccaaaaaacaaaattcttcttttggtttatattacgcatcatctttattacctataacttgaattctgtttgctctgcctcttcatattttattttgtgttgaaattctttgttcttccttgtatttgcatggttttagatgattctgtatttattgtcagttttttttaaacctgtcaccgagaaaagggaaagaaaaagtaagagtttatatgaccttttctatgattggagtcttcttcatagttccatgcgtgaaaatattccctcatcattatctttcttcttttttttagcatctttcttctgaagtactattgacatgtactagaattgcaaatgctacttatgcttcacccttttccccattgaagtgtctagcagtttacatttttttttactctttctactttccttttgaaaacaacatattcttaattttaatgtacacattagatttgtgtttgagcgtcatggtattacttttctttgtttatgcagcattaaaagttgcatgtcatatttgtaagagttgttacaaaatgatatccagattttcttttcaattatttatttttgtttagctttctttaattttttcatgcttgaagaaataaaattaatgcttatttgttttattcagataatgggaacgaatctcagcactcccaaaactgagaagtcttctgatgatggtgaaaatgagcatcttagatatggtttatcatctatgcaaggctggcgtgcaacaatggaagatgcagtgagtaatcactattaaagttgctagtttattaaattgataattttgtgtgtaataaatactcaattctgtctgcatagaaaaataatttatttagattttttttccagtttcattggaaggatagaaattttcacaccaggatttgtcttagttaaaccattaatatgctgctaagtttcttcattctcttttattttgctagcatgctgcacatcttgatctggatgcatccacttccttttttggtgtttatgatggtcatggaggtaagatttgaataattgtaactgtttggaagaatgaccgaatgccaggtttatagttttcttacagttgtcgtccatacatgttaaaatgattagctgtttcattattttttccccagttttacaactttgttacttttgcctctaaattaagaaaaatataattggtttttcttctatgcctctaaattgtgtttgttgaagttctatataacatatcatgctccacttttatcttttatcttttgcacttatcttttcaaacaaatagttgctggtttacctgctagaccatatctggcagaaattgtgcaactcatgtacctcaatgctcctctacttgttgcctcaagactttagtgatcccagccagggaaagaggagactaactacaagagaaattatgtgttattttgtaatcaacaatatgttatggatgcttaattaaccctaaaattttcaggtaaagtggttgcaaagttttgtgccaagtatcttcaccagcaggtgctcaagaatgaagcatacatagctggagatataggaacctctcttaaggaatcatttttcaggttagaggaaaatgagggggccctcatgtttgttactaaaagtggttcctctgtcatgtcttcagaatatagtgaccgaagctgtttatttgtttgcactatctaaattttaaacatatgattaatttattgcatttcattaaaattacagaattaaaaatcacacaaatgaagtatggcattaaaattgatattatagacaaaaaagtgttcactagttccttatccattcattgttttctgtcacttctcatccaaaacaccagaagaaaaaggaataaagaatgagtaatgcactagcattttggtgattattgcagaatggacgacatgatgcgtggtcaaaggggatggagggaattagcagttttgggtgataagatagacaagtttaatggtaagatagaagggttgatttggtctccacgaagcagacatagtaaggagcaagatgatacttgggcctttgaggaggtaggtatgacatttgaaggatcctttaacttttcaacatcatagctttatgggctaatacttgatttgttgatacctggcatatggctttttaagtaaagaaacaaggtttaaaattgtgattgtagttatggtttcgtcaaaatccttaatgttgtgagatgttacggacaaatgtggccacaattacggtcacaatgtcattgggagcccaaaaaccttgatgttgtggctaaaattgtggatactgacgattttgtaaaaccttgatgttgtggctaaaattgtggatcctgacgattttgtaaaaccttgtaaagtattgtgcccatggatttagagttgtgtttctccaaatgtgttagttttgtgtgtgtgaaactataattttctttacacctccatttttcatttttggtttctttttgttgccgttctgtaatacgtctacgtattataagcagggccctcattctaattttgctggaccaacttcaggaagcactgcatgtgttgcaattattagaaacaacaaactttttgtagcaaatgccggtgattcacgttgtgtagtatgtaggaagggtcaggtaacttcacgatgaaaattcttggattttcatcttcaaagtaacagatcttgtgtataattattaccatagttttctgactatggcttatatctacaggcgtatgatttgtcgatagatcataaacctgatcttgagattgagaaggaaagaatcgtaaaagctggtggttttattcatgcgggacgagttaatgggagtttaagccttgcaagagctataggtacttatctagtggcaagtgagctttaataaattataaatgttaatttaggctttcttccaattttaacctgaatgagtggcatggacctctgcacaatcagtcaagcatattgtgaacatgataaattgataattaagtgcaccaaaaataaagtcaagtagccaactttctatgattttagtttgtatgctttattttgtttctttacaaagcaagttgaggttgctatgcatgtgaatttgtttcagattggcaaattgagcattccatatatgggtatttgtttcttctggatgctaatttctttgtgaagctttcatatatgtttattttggatttttgggaatggtgtacaatggacatggtttgaatggtccccattcaatatgtttcaatgttctgcaggtgacatggaatttaagcagaatagattcctttctgctgaaaagcaaatggtgacagccaatccagacataaatactgtaagtttgttccttacaaatcagcttacttgacatgtgatacataaaatagaatttaatagatctaccgcagtttcccagttgcaatatggaagaaccaatagtgaaatgcttgcttaagagtcttggtatttaatactattgtaagttttcacctttaaactgatagttttggtttttacattttatgacaggttgaactttgtgatgaagatgaatttatagtgctggcttgtgatggcatatggtatgcttcttatgcttttataaccccatttcagtccttttcattaatttactgaaatgttgttaacacattggtctattggtccttgcactttctctattatttatttatttattttggggggatgtcatgcctagaaaattcttgagtggttgatttttttccttcttattggttcttggttcagggattgcttgtcaagccaacaattggtagattttgtacgtcaacaactgctcttggtgagtttttaagttataaaatcttcctaataagatggtgaagtattaaattaataatttgctgttattgcttgctgcccatagattatgcttttaggtgatatttaatacaggacttagtactgtcacgaataataattgttgtttgtaattataattagttggagagaattagtttctttttttttttttttcaaactggggatagagttatgttctgtgacataatgcaattttctacttgccattagtcattacctttttagtttttactcgatgcttttaggaattttttctagtataagtaaaatcagtaataggctgttaccttttttgtatatagtctgtgttatggcctaatgggaagctgatgcatcatttgtcacatgggcctaaggtttagaaagtctacacaaggtaaaatagagggagaacagggaagaataatgagctagcactgagagaaggaaaaaaattggggtggttactgattttgtgagttgctgttgtcagatccgattcctttaaaagtgaataactcacattagggggggtataaaatactaatcttagtcaacagacataggttgagtctagtggaagggactaatctcccacaacataatgaatcgattttcaaatgtttgttagaagtgtctacatttaatatctgaccatgttttgaatggaattgcttccaaaggagaataccggtagaaaaccaaaaaaagtaatcttagtcatcgatgaaaaaatatcatgaacacatgcataacaaatcataatgtgttgaaaaatgaactgttagatattttcatcaataacttaaattatttactttactctctgaagtgagttagtcaaatccgcagttatcatacagggacataggctgagtgtatgtgctggggtatcttttcaactctcagtagtgttttagttttcagctagcttttaagctatcaactagttttttagctacctatgccaatgccaaacagtggaatacacactctgtaggttaatgatccaagttattatttcaatgtggttttagctgatccataaagcagatgttttgaatatgataccattttatctcatcacaggaaactaaactttctgccgtttgcgaaagagtactagatcaatgtttggcaccaacaattactgttggtgatggatgtgataacatgaccatgattttggtgcagttcaaaaaactggcccaatcaagtgcaccggcataacataagagctatcatcttcaaatgaaataagctacatctgaactaagactagagagcagtgaaagttagtaaattttgagcagcaacactacacacatgtagacacatgagtctagatgatcattttcacttcaactagcatttgtttccaaatttctgtgtttattttcacacctagaatctgttttgcttgttcaataagaggctccaaatgttagtttttatagctattagcaacaatctgcaggcattttgtgatgtttagctacttgctatcaactgcaaggatattggccccaacttattagaacattgtgtatatatatagtatatctcatcaaataaccttgttatgtgatgttggtctcacaggtgaggctgaaac

SEQ ID NO: 3 - DNA, 4195 bp, glycine max
tccagttctacacacatctcgattctcccagacacaaattcactttcaacaattttattttttccatataaaaaaaggatcaaacttgagaagtctcttctgttcccaaccactcaaaatgtcctccatggcttcctccaccgctttcttcacacccctccatgatttcactcaagccagaacaaccccatcaccccctttgcctttcatcaaatcctcctttgcttcctccaagtcaaccttttttcaccctgccctttccctacaaacctcttccaactttcccaggctttttggaaagcctaagtttttctctgttcatgcaagggctgccacagaaaaaaccatttatgattttactgtcaaggtaccatgccccaatttcagtttccattacccttttgcttgaaaacaaatgggtcagccttgaaattcgaattggaggagtggggtgtctaaatttttttttttttcatgacaatttgttctatggatagtttttaccgaaaggggagcctttttaaattttagataagaaaagtttattattttattatttttatttgttatgtgctcagggtgtttattatattatatggtgcaggatattgacagaaaggatgtttctcttagcaagtttaaggggaaggttctattgattgtcaatgttgcttcacgatggtatgaattatgcacttttcaagggagatatgtagctcaatatgtgtcatggagccctattttcttctttgaatttcccaatttttaatgtaaaaaatgaaaatcaaataagttgtagcttaacatttgagtcaaactttctcagtttcttgtcagttatgataccattgaactaatttccttcctatgacagtggtttgacatcatcaaattactcggagctgtcacgcttgtatgaaaaatataagaatcaaggtatgctatgcataaagaatggttatttgtgtatcttcttaatatttctaagtatgaattggtcatgttttacttggtgtttgcacatgtatcttcttggttctgaatctgtctacttgtgcaaatcattctgtacttgttggatactgtataataaataagagagaagtgtgtccctcctatttgaattcaggcacaatttattgacaactttagattggatacctgtgtcagttgtttaaatgaagtttactaactggggtctgtttctcaaatgttcccctattctcctcaaagacaccattctattgatacggtcacttcaaatatcacatctttgtatgggcaactattgattatgattttacaatgtatatcgatttccctgtgtgatgcactgatgcattatacaaagtctgctgacaaactatcttgctgtttttccatgtttatcgaaatgtttatgatgtgaaatgtttcagaagctgctaactcatgtgttgcagtttattcctcatttgttgttattttggatttaattattaggtataagaaccatctaaggctgaagaagaaagcgaacatgtttttatgaattatttatttgacaacatgtttatgtagaattttgagtatattttttctccttattcatatgaagatcaaagtgaacttcttgcagttgaaggttagaacccagaaataaagacatttaaggattccatcctttggaatttaagatattagcttgtatgacattattgtattgattccccctctccattttcccaagggcttgtttgtgtcatgctacctctacatgtagctaaattaagggtgttctgtgtgcatgttagggaaggtggaaatttgaagagttatgtaggggagtgaaatatattgcatttcattccaagttggggtggaagaatttacaaaacgatagaatgcctattgtggctgctccattttcgttttgatctgtcattcttttatctctaccaagcacacctttagtttcttattaaaatgggtacaagacaaggttacattatggtggaacatgggtgaagctttggccttataatttctggtctctttatacttgaagaaccttggcggtttcgttattggaaaattcaaatacttccaacggacagattcaaaagtgacttaaattcaattctatattttgttacttttataaatatggttctaaggtttttagttttacattaatatgttttctcagcacacacttctagtgcccttattctgcttcattccaggtttggagattttagctttcccctgcaatcaatttggtatgcaggaacctggatcaaatgaagatattaagcaatttgcttgcactcgatataaagcagaatttccaatttttgataaggtttgataattttagttattagttcaaaacagttttaatctgagtaaacaaagtattgaagttctataaaacaggttgatgtcaatggaccatttacaactccagtgtaccagtttctgaaatcgagtgctggaggctttttgggtgatcttataaagtggaattttgagaaattcttggttgacaaaaatggtaaagttattgaaagatacccaccaacaacgtctccttttcaaattgaggtatgttaagcttcccccaatagtaaatccagtttttaattgttaccatgacagtgaaatattaactaatgaagtatgtcaatgtctttcctactcaagattgaagcctgaaggtgagtataatagagtatctgttaatattgagaaatttagcattgaaaattcagtttgaaacttttaatgctgacaacttagcactagaatttagtgtttgaaaacttgatattttaagtgcctgatgaaattagtttatgcttaatgtagtcagtaacctcttgcatgctgagcggtttaagcagttttcacttatatttattgtaagtaggtattgaacaaaaacagtaaattattttcagcatagttgagtttggaaatatgccctacatctgagatagtgttaaggcaacaattttcagtgggtcagttctaaattaagttacactcttccattaaattgtagcacaaaagaatcttatatacattgttgttagaattgcactatttgatattatatggtaagccattgatctgtttcatatcatgtgatgaatttgattagaactttaggagaaccacactacaatcattgtaaatcccacactagaatttgatatgtccaatatgggactcaaattccatagcttgccattggatcctaagagattcacatttgggtttgaatcatgtgatacatattacatacttggattggcataattctgtatcactgaaaatgctcgaagactttattttcttaaagcattgaggtgtgcatccattgaaggcgttaggagagggatgttaagcccaaaaattctagaagggtgacattgaccaaataaagccaacatgaattattaccttgagtcttaaagcattcataatttagaatcatatggttttttcctattacctctaccttattatgaaaaagaaaaataaaaaataaaaatccattttctaccacattcatatacattggattagtcatgactttttcagttttcacaaataattttagatgtctgtctgtctgtctgtcagctagctgtctttgtttagtaatccataattataggaaagttttgagagtgattaacagttaaaagaaaccatctgttacatgtatatacaagacaagttatatacttttatctgtatgtttgataattgatactatagctagctatcaattttgttttttattattaacttgttcttgcctgatttatattttgtcagaaggatatccagaagttacttgctgcctgagtgtaattcagaacacaaacgagagctgtatgtaagagcacgaaccgagtgatgtgtgcacaataagttaaaactgaagatgagcaataagtatagctatgcaatttactttggtgtctttactaaagaaattatcagtttagggaagaaaattatactgtattaccccaaaatgattgaatatagtatatgtttctctctttctttttttctttttgtggaagaattctctggccatttatattattattctctaagtaatatgctttcattgagaaagttcttaacttatgtagttaaatagatatatatgtcacatgttggagttgcactttaa

SEQ ID NO: 4 - DNA, 6766 bp, Glycine max
gaagatgttgaataagtttgacttcaacccatggccacattatatcttcttcttacacaatgttggaagtcaaatcaaatctcccatctcatcaaaacccagttccctcccttccccatgaaacagagccaatgggtgagaacgataacaaaaccaaaaaaaacctcctctccctcctaacagagccaatccaatggatccaaagtctctcctcacagctcaacccaaccttcgtaatcggcgtattcataatctacggcataggccaaggcttctccggctcgctcttcaaggtagtagtcacacactttttcttatatccaaaaatgttagttttgttatcttgaatccaagattttttctttctttttttctcttcacaattaagttaacttttttctttcgataaacgttaattattagggaatatttgaatcatgatcttggttacaggtagtctcagactactactggaaagacgtgcagaaaattcaaccctccaccgttcaactctacgtcggcttttacttcatcccgtgggttctcaaacctctctggggaatcctcaccgacgccttccccgtccgcggctaccgccgccgaccgtattttattatctccggcgtgattggcgccatctccgccgcggttgtcgcattcgccgggaacctcgccgccgttgccgcgctgatgtgcttcctaggcgtgtcggcgtcgcttgccatcgctgatgttacgattgacgcgtgcattgcgaggaacagcatcgagatgaggaagctcgcgccggacctgcagagcctctgtgggttctgctccggcgccggcgcgctcgtcggatacctcgccagcgggttcttcgtgcaccgtcttggaactcaggtacaagagtagatttcaagaatttttcagtgatttagtgctcaattgtcaagaattctacggttctatggtttctaaattgaatctagttatgttggaataggcttgatccaattgtcacgtgttattataattattaattactaaatgttgttgattgttttaattgttctaagaaatctaattaaaattcactcactagttgcttaactcttggcataattctctcaaacataattattttaaaaatcatatttaaaatgatttttttattaattaatttactaaagtaaaaaaatttaactttacaatacatgataaataaatttttacttcagaaggaatatttgaagtatttttaatcggttcatggtataaaaatctttaatttactttgtattaaaattattttttatgattttttttttaaatttagttcttatgtattataaatatttttactattaaataattaaaaatcatcatttatataatatttagataaaaaatatttattatatatgatgactgataatccaatgccaatatatatatatatatatatatatatatatattactggttgttgatagattatttattcattttcaatatgttgtagttgtagagacaaaaatgataattatatttttttaaacacttcttttattatttaaaatttattttgtaaaatttatttgcagtttactgtaggggataatttcagtgaatttcaagttgaaaagagagtattattaaagtgcaaaactatgatcaataaatttaagggtgttgttaactagaatgttaagggtattagttggataaaatatttattgtaaaaatgaaaatgtaaaataacttataaataacataattttatatatcttaataattatttgtttcttttagttttttgaagttttaaaaatattattgaatctaagatcttctttgatgatttcctctaaattcgttttgagtcttgcctgactttaaatgaatttggatcattgatatgtttattatttgtgtattttgtaggagtcgttgggtcttatggcgctttctccggctttgacaattgtgctgggttttgtgatttatgagaacagaacaagtgcgtcgcatattgaaaagcaggttagaaaactgtttgtccctttttttccccttttcttgactttatgctaaataggagtataatttttcttacgaaattgttatagttaatacatgttttgagctaaaaatatttcaatctcatcattaaaaaaataaaattcaatctacaattatcggtgttagttaactagtgtccatacccgacattgtctggtaatttttacatttaaaagtttattcatattttggttgtatatgtatacgtgtattagtatgagcacctaaccgggtatgaacaaaaaatacgctgtattatttatttatcatatgacaattttttttttttatataaacgcatcaaacaacttcaacatgttgaaaatatattacaattatattttgttaaatatttcattgtgcataatatttttggcagtgaataatcttttttgtctttatcatatattaacactttcaagtcaatcatacttttgactcttgatatatagcaacatataattatttatgggaaaaaataggtcacaccaacttgagataaactctgtcctgcccttgactcttgtttattgtcataacaaaacaaaccatcaatggaaattgttttcattgatatttgaatggagtcatatgatatgaagaaattaaattcaattgaggaatataaaccttattgtcaatgttgtttctagttataataacaactttaattatgtttacgcctaattcttctacaaccaatcctgttccattacacaatcctatagatatatctgcgtattaatattattgtatttcactttaaaatatgtattatcttttaatcagaaaattcagtaagaataggagtaaaaaattactaagcgatgtagatattataaaatcttacttctgagtcaatcaaaaccatctcagttggaaatgatttcttgggctctccaacaagatacatgctacacattggaataactcatgaatttccaagcttgtttacccgagcaaacatcacttaggcgtttaaacataacacacatcttctaatactttcaaattgattgagagtatttggaaactttcatttttgcttagaatgagaatgctatttatagtagtttcattgagtttctcaaccattcagccatattttaccaccaccggaagtagcgaaaattcaaaacttctaccaaactctatctaatagtgaaggttggaatatttgaaaatgtttccaaatcatcccacatcagtttttctactaatctccagaaatcacgcacaagaagattctgatatgacaagctttacatataagaataaaaagaatgaacatatgcactgctcacaaagaagactctaatgtgaaagttagacattggtttgacaatttagaactcctattttgtcgatgtactaagagtataataaatttagttttatttaatacagataagttgaactgctttgcttcggaagacaagactgtgcacatctcctttaacttttgcagcatattgaaaatgttcatataaggaaaataataaagaacaaaaacaatagaagattctgatatgaaattaccatttgcgattttctgtttcttaattggaaatataagatgacacaagaaggtgactaaaggaaatatgtctacaacacaaccttgagtagttctcatgcactggttccaattttctacattccaattataaataaaagtgaataattagaatttttataactctacgtttagttccaaaattatcaatcacaaattagatttttttttacataaaagttttaatgttacaaaataaaataaattcatataggatataatcactatacaatatgtatgtattatttaaattcttttaaaactatcttgcaaaagatataatagaacttatataaattaaatgggtttttttttctagcttggaagtgactaccactaacaagaggataccaccaaagaaaaatgtcacaatccaactaattttccgtttttccaaaatttaatgattggaaaaaattgcatattatcgaaaaaaagctatatatactacataaatgaaccacattacattaaatgaaacacaacttttccaccaccctaagttataagcctaaaattattaacctactttccactatagaagaattctaaatggaataacttttatctagttagtttgtattcatagtcactatttattcattagaaaactcaaacatcatgatctctgctttgttaatattgattttgtaattttgaaatattatgatgaaataacaccacaaagtcataaataaaaagtatatattatataattagttgtatccgtaggatgttctaatttcaaaagtaatctaaatataaaaaagaaacaataataaataaaaatagttaaaaggattaatatacaacaccaaaaattatgaaagatctaattctacaataaatttgttatttagtacaggagaaccattaaccgaggaaaagtggaaaaatctctgttaattgaatagtgaaatatcccattcaatcttttttccaataacattgttgtgttataaattagtcttaattagttcgttgttaattgttagaattgtttggttgttagaattcttataacttcgagatgtataaaggagactgatcgaaggaagaaaattcattagttcatcgttcagaaaagaaaatcaaggatcttcaaaggcagagatctacctttgattatgataaagaccattttatcatcttagtcgtgcgctcttttctcattaatcaacttctatttttagaactacacggtttagtgttctttttctttactagttaaactgtgatataagatacgtactccatttctttaaagtatattttaaaacatgcacattactcacgagtcagacgtcaatgttctcaacacatagaaaaaataaaggtcttgtcattggcgggtgtgttttgctgtcatgttccttttgtatccttccaaaacattttataattaacagtgttagtattcttttctagagttgatgactacgaaactaatttttaattttctggatttgaacttatttgttgtttaaagttgggcactttcgttgaacaatctaattatactactttatttgatttttttaaaaaaatgtatattatcatctttaggttaaaaaatatctcgtattcatttgtataaattcatcaaatcaaagactgattcttaacccgtatactagtggatccaggtgggcccctaggttcctgccccccttagattatagtattatactcttcaaatttaaccattctatgtatccatttcttcatctgtattttccttgcaacttgtgtgtactttttcttactttgatctattatttttatcgctttatctactcttttgcatgtccattttttttttgagtttgccaatcaattagcaaaaagcattaagatcgtaatactaataagaaataactaatggctaattttatatcaaagaaattttagaaatgattgacacatactactgattttcctatcttgaaagatttctatgttagctagatgatctaactaaacacccaagatatcgcaggctgtggagagtgtaggaatgaagattagaagcatgtaccaaacaatgttgtatcctcatgtgtggaagccttctctttacatgttccttgccctgactctgaatgtaaccactcacgaaggacatttttactggtatacagatccaaaagccggccctgcattctctcaggtacccctcatattttgtttgcaagtaactaagagtagatagtccatccactaaaaatgtaagagacttttatgccaccattttccaatcacaaacttttatatagattaagtttgttgactttcacatgaccatcttacgagtcatgcttactgtgaaaataaaacagaccgtgtataaaaattaaaccaattttctttgcaaatcataacttggtttccatatgcaggagtttgtaggggtgatctatgcaattggtgcagtggcttcattaataggggttctaatctaccacaaggctctaaaagactaccaatttagagacctagtattctatgcacaactcctatatggcatatctggtgtgttggacctgatcttcatcctccgttggaatttggtcattggaatccctgactatttctttgttgtccttgaagaaagtgctacaagaatcacaagcaaaatcaggtggatgcccatgatggtactcagcacccagttgtgccctttaggcattgagggaaccttttttgctctgttaatgtgcattgatagcattggtgcccttttctccaaatggggtggagggatgcttcttcgtgtcctccacatcactagaactgactttacaaacctttggctggctgttcttataagggacatgcttaggtttgctacacttgctttggtttttcttgtccctaagacagatcaatatgaggaactgcttccttctgaggtttcaggaaagaacacgagtgaaaaggtacatgaagaagagactttggagcttgtccccatccatggcaagactgaagtttagataaaatttgtcgcaattatacacacgtcaacttcactatgcaattacctatatgcgaaattgttagatgcatattcttaggtaataacgccgtgataagcactaacagatttacacgtttttttttttt

SEQ ID NO: 5 - DNA, 3207 bp, glycine max
gccaacatggtcatttggtgaaatgtcgaaattagcaggagcaagcgaggttgactgacttggcatattcatataatcatattctgaactgttttttcagtttcgctattccactctgctgtgtgcgtgtggtcggtcactgcaaacgcaaaaaccagaacattctcttcagcttaacccttctttttcgatggtaaggtttcccttcgattctcgctaacctccttttcaccgatcccaattccatttcccctttgcaaccaattagggctattcctctttttccgcgttcccaattcgttgcttacgtgtcttcctcgcgcgattcactaacccttgcttttgttttcgcaattttttgcaggctaatcggcacacgattattcttatgcagacctcgcagaacagagcaactaggacgttcatggattatgaatccataactcaggccatggatggtattaactatagttaatacttgtactatagtcgtagttctattttttcatttttttttttaattttggctcgtgctattgttttttcttgattcactgtagatagataaatagatgcttgtgttcttgcttttcttttttctgaatttgcatgcaaatttgatttcataaattgaaaaggacagagtgtaatttggcactggtattttgtttttgagttgtgctgttaaattaaaattctgtttttgtggttggaagttgtatttttttttttttttgtggtttaaatcggctaggattcttaccagtcactactactaagcatgtgaatctgttggtgttcactgtctccttgagcaggaatatggcactaatctaagtttgatgacttaatgtgcatgtgctgcttgaatagattgaacaacgaccaaattcttttgaggataattccagatgacttgttgctaaccttcttaggggtctataaagtattatgcttttcccataagattggctttatctgcttaccttaaagctttgcctatgtaacatttttttttgttttatttgtgacttttctctaatgcacctatgtgaatctcaccttagctactttggtttgggcgtgtttgacaagaccagtcgaaaccctgttgtaatgctgatgatcatagaccaagaacccaaggcaacagattgctgttccctgtcttcttaattttgtactcttaccaaacctttttacctgtgacagtaatttgaattcatcccattaaaactcttgcttgaatttgctctagagcccatcgtatgcaggttttgttcatggcacctggacaggcaccaaattctaaccccttagccccctcctatttcttgaaaatcctcattgcaagctagaaatcgaatgctgtttagcaatctatcccaatctgttgaaagctagaaactctattcagcatactgaaccatatctgggtagccttgatgattcattgcagcttactgaaccagcatttgggcaatgcaatcagctgaagagagcaatggatcatgttttcttgctattttgttggttcaagaatcaaataaagccatgaacaccatttatttttacttttcatttaatgtgaatttcattttcatagaaaaggtgtcacattgtttttgcttttaaaggagtgtccagaagttatcaaatgtagatagacacacaaaaaacagtggtatgcatctacggcaaacttagaaatatagcatgttagaaaggttacaaatagataactgatacttgctgttgatataatttctttttctttttgtggtacaactacaagctagtatgattttgttttttagtttttttttttatagatatttcatagggactcattctatttttattgattctttttatggatcaatttttagcattaaatatatgttttaatcttgtgcttgtacaggcatatgtgcactgtatgaaaggaaactgaaggagttgaacccgaccataagaaatctctcgtatgacattactgatctctacaacttcatagatggtcttgcagacatgagtgccctagtgtatgtccctctttctcactctcaaaaatcatgtgcatgcatagttcattaattcagtcagttttctttaaatctaattttcatttgaaaggcactctcactcttgcagtgccctagtgtcagctttgttgatatttctgttaatggtttaactgatgtagagtgttgacatcattttcctccatgagtcgagaaaattaagattatgggtggaaaattggtagcaataacgagtcttaacagagtatcttgtttattgctgctgttctcttattgatgaaaaaaacggccataaaatacattttgaatattttgtttataggtctgtatcatatgtaagtgttggagaaaatatgatttctaaaccttctccttctgttcctcttccttcccgtctgccctagttttgtgcctagagggtttataagaacttaatgaatcacggtaattgctgtatcgtatgtttgctctaccagttctgactctcgggaaaaaatatatgtaaagttgtgtaacagcttatgtcaaatgtctatgcatatagtggtttttatatatactcggcttgctctgcatgtggcttgctttggcatcgattaatatgactgtttgtttggtttctacctgcaggtatgatagttcaattcacgcttacatgccgcacgatcgacagtggattaagcaacgaactttccaacatctgaagaaattagcacactgattaatcccttaacttttggcaatttaatgtaaagaaaatcgagaatggccgagagcctgtctgttatgtgggctatatactccgttggctatgtaatacgaatattgcaaatgctggcaggtaattataatatcaacagctggtactgctttagtttcataaacaaatgattgtcgttggcatggttgtgtatcttctatgtctgcaagacttgtcttttttaaaaatcttaatcttgtagttgtaatattaaatagtgagttgtactgttgaattagttatccacacattatgattcacaatgtcgtatgaatgctagttttgagagttcttattccgcgagtaatttttcttgtagtggcaatggaaaaatatagttcagttttaggttgtgaggtgtggcagtaatattatttgaaatatctaaattttggttcttgc

SEQ ID NO: 6 - DNA, 1854 bp, Glycine max
atgttgcccttgaaaacttgtcttcttcttttgcccatctttatgatccttcttccaatagttcaaccactctccttcaacataactaactttagcaacaccgaaagcgcaagccccattgaatacgccggcgtagccaagactgagaacggaaccgttgtgctgaacccactcatcaatggcggagttggacgagccatctgtgtccaaccactacgcctgaaaaaatcttctaacgaagatgtcacagacttttcaactcgtttttcattctctatcaacgcgcctaataaaacaaactacgctgatggctttgccttctatgtggcaccacttgccttggcgtaccaaattccacccagttcaggtggtttacgtctcgggttatacgatgacagcaaaccccaaaatagcttcgttgctgttgaatttgacccttatgtaaacgagttcgacccaccagtgcagcatgtagggatcaacaataactctattgcatcccttgattataagaagtttgatattgagagaaacatagggaagatggggcatgcgttgataacctacaatgcttctgccaaactcctttctgtctcgtggttctttgatggaactagttctgacgctaattctctttcgcaccagattgacctaggggaaattattatgtcagactgggtcgctgtagggttttcgggatcaaccggcacgaccaaagaggaaaatgttatccattcatgggaattcagttccagtcttgatttaagttccaccgatccagaggtaaacaacgaaaatgatgatgacaacaagattaccaaatacaaggttcaggtcaaagtagtagttgttgttgcagtggtttgttctattattgttgttatcgtggttattagtgttacttggttgatcatcaagaagagaagaagcggtgatggttttggtttggatagagcagctattccaagaaggtttggttataaggaattagttgcagctaccaacgggtttgcagatgatagaaggctcggagaaggaggctatggacaagtctacaaagggtttctgagtgatttagggcgcgtggttgcggtgaaaaggatcttttctgatgttgaagattatgaggaaatattcacaaacgaggtgaagattataagtcgtcttatgcatagaaaccttgtgcagttcatggggtggtgccacgagcaaggggaggtattactggtttttgaatacatggttaatggaagccttgacactcatttatttggtagcagaagaactttggcatggggtgtgagatacaaggtggtgttaggtgtggcgagagcacttcgatatcttcatgaagacgcggtgcagtgtgttcttcatagagatattaagtcaggtaatgttttgttggacacagatttcaatgctaaggttagtgactttgggatggcgaaattggtggatccaagattgaggactcagaagacaaaggtggtggggacatatgggtacctggctcctgaatatgtaaaggaagggagggctagcaaggaatctgacatgtacggttttggagttttggctttggaaatagcatctggaataaggacttaccgggatggggagaataatcatgtgcctttgacgatttgggtgtggaaacattacgaggatggaaatgttttgaatgttgccgataagggattgaacggtgattatgatgtgaatgaaatgacttgcttgctcactgtgggattatggtgtaccctacaagaacacaagaaaaggccaaatgctgaacaggttattagtgttcttaagcaagaaaaaccattgccagtgctttcagcaatatga

SEQ ID NO: 7 - DNA, 2287 bp, glycine max
atggaagatgatgaagatatacaagccatgtttggacgcttccaaactattttaaatgaattacattgtcttaaaaaaacttttgataactattataacattgacaacattctaagcagtttatccaaaaagtagagtccataggtgacaaccttgagaaatgtgaaaaacctggaatccatgtccattgaagagttgattcgaaccttaaaggttcatgagcaggaacttcaacaagatgaaggattcaaaaagggaaaatccttggctctaacatcatagaaagctaaagcatcttcaacatcaaatgaatattcatctagacctgcatccagaagctcatccaaagttgtgaacattgatttatcttctgatggtgaatccaatgatgatcaattagctttcaactcaaggaacataaggaacatgtggaagaaaaggaatggaacaatctggaaaggatccaagaaaccacatagagaaagaaaggataaggagaaaagttccatcatatgctttgagtgcaagaaaccagggcatttcaagttagaatgtctagatcttgataaatcagcttacaagaaaaagtacttcaaatcaaaggacaagaaagtgttaatgaaaacatgggaagaactggacgacacattgttcgatgtagaaatagaagaggaagaagaaaccaacctttgtttgacaacaaacactgcatcaaaaggatcagattcaaaatcagatgaagaggtaaatataaacaacctttaaactctttaattatcatatcatgaacttctccccaactcatccattttatccaaagcttatcaaaatttgagaaaggaattcaaaaatctttcaaaagattagaaacaacttcaagggaaacctaaagaaaacatagataattcttcagaaatttccactcaattatctgatgactttgaaagcctagttagagacaaaaaatcttcaccttgagaataagaaaatttgtaaagaaagatctagtctatagcaagacctttagaagttgacaaataaactaaaaggcttacaaaatgagtatatcacactcaacaaacttcatgattacctaaatgaagaaaggtgtaatccattgaaagaatgttcacaagtccataagaattatgaaaacttggacgcaagtaaacatgggttgaatgtgaaggatataagaaaagcttgaaattctcaaatgataaacttgaacaatatgataaggctctccaaaagcaacctcaagatgtagttatgcttcatcaagagactgaaattctaaaagagactttatccaaatttgttggacgcattgagaaacttaacaaaatgttaagatacaacaaattcccaattgatgaatttggaaatggatataaaggaaagaaatatgttcatgatgaagaaaacatagtatgttatttttgtggaaaggttggacacatgacatccaagtgtagacatctaccaaagattagatcgtccaatgcttttgagaactaacaagaaaggacctgagaaaatttgggtacctaaagaaaaaataattcatgttgtagatttttacaaccatggacacaccaatcatggtacctgaacaatggttgctcacgtcacataataggagaaaaatgtatgttccaatgtctgaatgttccaatgtctgactcccatgcatggtggaacagtcactttcggaggaaaccataaggggaagatagcatgagtaggaaagaaaagtattgacccctatcctcccattgataatgtactttttgttaaagggcttaaacataatttgcttagcataagtcaattatgtgataatggacttgatgttttctttagcaaagaagggtgtgtcgtccaacacaaaaatgggacccaactcttcattgctaagaagaatggtaacatgtctattatccatcaaagaaaattactaggtatggcacaagaaacttgaacatgcaagcttgaggttaatatcgaaaaaccaaagcaagaaagtgttcgggttttaaatcatataaaataggcactgataccaaatttgagataaataaatttatatgagtttaaaacaccgacacaacacttacttgttgaagacatgatctagaagatgagaattgtctatgcgtagtgcgacctgcagaaacccaaaagtcaagtcttcctttacctgatcacactcttcttattctagagttttaaatggggaaaaccctatagagcaattgataacctaacgacaaagggggcctag

SEQ ID NO: 8 - DNA, 2350 bp, glycine max
atgttactgttagctaccacgtcgtgtattccctgcttgaaaacttttcttcttttgcacatcctcatccttccaataattgttcaaccactttccttcaacataaccaactttaacgatactgaaagcacaaacctgatatttggtggcgaatccaggattattattcagaacggaaccattgttctcaactcagacatcggcaatggagttggacgagccacctatggccaacctctgtgcttcaaaaactcttctaacggacatgtcacagatttttctactcgcttttcgttcaccattgatgtgtccaacaaaacctttttcggtgatggctttgccttctacgtggcacctcatccctaccagattccactcaattcaggtggcggccgcctcggcttatacgatgataacgcaccggcaccacacagtaacatcgttgctgtcgaatttgacacttatgtaaaccgttatgttgatccaaatatgaggcacgtggggatcaacaacaactctgcgatgtctcttgcttatgacaggtttgatattgagagcaacatagggaagatggggcatgcgttgataacttacaatgcttctgccaaactcctttctgtgtcgtggttctttgagggaactagttctggttttacacctaatgctaattctctttcgtaccgtattgacctaggggaagctctaccagagtgggtcactattgggttttcgggtgcaaccggctcgttaaaggaggaaaatgttattcattcctgggaattcagttcaaccatgaactctatgcattcagaggtaagcaatgaaaatgatgataggatcatggttaaatacaagtttcaagtcaaagcagtagttgttgcggtgatgactacttgtgttttttttgtgctcgtgattattggagtttattggttgatcatcaagaagagaagaagtgaggatggttatgatctggatagggaaactatacctagaagattttattacaaagaactagtagcagccaccaatggatttgctgatgatagaaagcttggacgaggaggctcgggacaggtttacaaagggattctgagttacttaggaagggatgttgctgtcaaaaggattttcactaacttggaaaattccgagagagttttcatcaacgaggttaggatcataagccgtcttatacatagaaacttggtgcagttcatagggtggtgccatgaggaaggagagtttgtgttggtttttgagtacatgcctaatggaagcctggacacaatctctttggggagaaaaaagctttggcgtgggatgttaggtaatgttagctattttggtttaataaattatttggtttacatttaaatttctgtacatgtacatgtacattttaactaattttaatccttatatatacatttcttaattcattttagttcccacaattttagatattaaggacgaaaacatattataaaaagtgtatgtatataaggactagctaaagcgtctaacttatagatgtagaaattaaaacataaagattataaggtatagcaatgaaatgagtaattaaacctagttatacttttatcttcttttatatgtttttttttccagcttaacatatttatctactcttattgaaacctttgcgatattttacgttctcaattctctttacaggtataaggtagctttaagcgtgactttagcacttcgttatcttcatgaggatgcagagcaatgtgttcttcatagggatattaagtcagcaaacgtgttgttggacacgaattttaacaccaaacttggtgattttgggatggcaaagttggtggatccaaggttgagaactcaaaggacaagggtggtggggacttatgggtaccttgctccagaatatatcaatggaggcagggctagtaaggaatcagacatttatagttttggtgttttggctcttgaaattgcatgtggaaggaggacttacaaagatggggagtttcatgtgcctcttgtgaaatgggtgtggcaacaatatgtgggagggaatgttctgaatgttgttgatgagagactaaacaaggagtttaatgtgaatgaaatgacaagcttgattattgtggggttgtggtgtactaacccaaatgacaaggaaaggcccaaggctgcacaagtaatcaaggttcttcagcttgaagcgccattgccagtgctaccacttgatatgcatgatcgtcctcctccttctctagctatgcacgcacaacaacccactaattattcctcacaatcactacctttcactaacagcattataagtgttggacgttaa

SEQ ID NO: 9 - DNA, 2340 bp, glycine max
tcaagccaagtctcatatcactcagccgtaacaaacaaaccatgttggctacctctgcatgtttccactacttagaaacttttcttcttttgtgcacccttatgatccttcccattgttaaaccacaaccaccactttccttcaacataaccaatttcaacgacagtactggaatcgccagcctcattggatgtgtaggcgtagccaggattggcaacggaactatagtactcaccaactcactcaacaacaacaacgacaacaacagtagttttgattttggacgagccatctatggccaacctatgcgcctcaaaaacacttctaacggacatgtcgtcgtaacggatttttctactcgcttttcattctcttcattctccatcgatgggtccactgaatccgatttcggtgaaggcttcgccttctacatggcacccattgcctaccatattccgctcggttcaggtggtagtcgtcttgggatatacggtgacaaagtacacgaccctactaacattgttgctgtcgaatttgacacctttcaaaacgttggttttgatccaccactaaaccagcacgtggggataaataacaactctgtggtttccctggcttacgcaaggtttattttttttttttaattcaaaaatattaattattagtttattagtttttgttaataagaggcatggaattcaccatattaccctccttcttcttcttcttcttcaccacttaatcaatcttataattcgtgtcaggttggatattgagggcaacatagggaatatggggcatgtcttgataacttacaatgcttctgccaaactccttgctgtctcatggttttttgagggaagaaatagttctagttctgcgcctgaagcttctctttcacaccagattgacctcggggaaattctaccagagtgggtcactgttgggttttcgggtgggaacgggaattccaaagggaaaaatgttattcattcctgggaattcagttcaaatatggatctaaaatccactcgtaatccagaggtaataaacaaaggaagtgacgacataactaaatgtaagtttcaagtcaaagtagtagttgttgcagtgacttgctctattattatttttgtggtcatggttattagtgcttcttggttcattatcaacaagagaagaaccggtgatggttttggtttggatcatagagcagctattcctagaaggtttagttataacgaattagttgcagccaccaacggttttgcagatgatggaaggctcggagaaggaggcactggacaagtttataaagggattctgggcgatttagggcgcgtggttgcagtgaaaaggatcttttctgatgttgaagattcagagagaatgtttacgaacgaggtgaatattataagtcgtcttatacatagaaaccttgtgcaattcttggggtggtgccacgagcaaggtgagctattactggtttttgagtacttgactaatggaagtcttgacactcatatctttggcaacagaagaactttgacatgggatgtgagatacaagatagcgttaggtgtggcgagagcacttcgatatcttcatgaagacgcggagcagtgtgttcttcatagggatataaagtcagctaacattttgttagacacggatttcaatactaaggttagtgactttgggatagcaaagttggtggatccaagattgaggactcagaagacaaaggtggtggggacgtatgggtaccttgctccagaatatttaaacgaaggaagggctagcaaggaatctgacatgtatggttttggggtagttgtgttggagatagcatgtggaaggaagacttaccaggatgcggagcataatcatgtgcctttggtgaattgggtgtggaaacattatgtggaagggaatattttgaatgttgctgataagggattgaacatggattttgatgtggatgaaatgacatgcttgctcactgtgggactatggtgtaccctccataaccacaagaaaagaccacatgcagaacaggttattagtgttcttaagcaagaagctacattgccaaagctttctacatgaaatgagagtgcaaatgacggagttacagtgacgtgagaggtcgcaaatgagagtgcaaaagagtttgagtgagagtgatgacaatgagaggtttcaaagttatcgggcttcagggttttggtggacaatatcaattttcttaaaaagatggatgttaaagaagtaagttaacatcgattttataaaaaaaaatagatgctgagtttttttatgttttcagtaacatagatt

SEQ ID NO: 10 - DNA, 4151 bp, glycine max
ccaactaaactaacgacaagtcaaatcgtacttctcctttcattaaactatattttcttcacgatttcctctaattacagacatagccccgcattcgacgaaacgcaacctcgttcattgcaactcctctgttaaacttgtgagtcatttcatttccaaacgctttagaggcccatcttccaatcatgtgaattttgtaatttgttttacaatataacaattggggttttgtcaaaattgttacttgctgtagtttgatctgttaacataggaactgaagttattatagtagctagatgatgatgatgatgatgaatacgatgatgctttttgctccgcctttttccctctgatgttgtttggctttacctttttggactaatgagtttatgattttactgttttttatttgttagagattagagatatgcagaagtgttcagaattgcttgattttactattgttcaattttaatgccgtcatgatgatgaaatctaagtttgagtgtggaaaccaagatgctattgttgatgatggagaaattagtttgaataatggtcagctgctactgcaagacattcaagaaataagcaaagctctttatgccccttctagaccttccttctcctcagttcataatcgatccaagtctgcgggaaaaacccatttgtcgaaacctcaagttgccctaacccccggatttctaaaggaagatctgtttcctagggacaagaaattgtcttcagcatggaattggaagaagcctttgaaggttttgacccacattggtggtcaaaagttcaaatgttgctttaaccttcatgtccactctattgagggccttcctttgagttttgatggcattaggttgtgtgtgcattggaaaaggaaaaacaacattctgcagacacgcccagcaagggttttccagggtgttgtggagttcaatgaaactttgagtcatggatgttctgtttatgctagtcgtgctgtgtctggtcactctgtcaagtacgagtctaagcgtttcttgatatatgcctctattgcaggggcacctgaacatgacatagggatccatcaggttgatctcacaaggcttttacctctgactttagcagagttagggggtgatagaagttcggggaaatggagcacaagttttaggttgacaggaaaagctgtaggagcgagcctaaatgttagtttcagctatcaagtaatgaagcatgagttaatggaatttggtggtgataatttgaatgttctcaaccttgttaatctgaagccaggcaggccaagcagcacaagcagtgttttggattttagtcctatcccttttcattctgatgatatgattttatcccgtgaaacattgatgaattccagctcaagtctttccaaatcaatcagtttcctatatcaaaaacttgatgaggggaacatccacaattcagcacaagcagactcggagcattttggaccacttaaatcccatgttgttacagaatcagagtcacctctagaatcaaatcaacatgaacctgatgacaatgaattttccattattgaacaagtagaaacattggaaggggattccttggaacttggtcagataggaaatcaaacagttgatttgtcaacagttgaaatcattgatgtggatgatattattaaggaagatgacatatttattgacaagaacacgagatttgattcaatggataacatatgcactagttgtgtaaatgacactatggcagatgacagcaaacataaacgcagtagttcatgtgtcagtataacttgcatcaaggatgctgatatacttcctgagacatcaaagtttcttgaccaagaatgctaccttaatgttaaatcaaattataaatcacatagaatggaaaaaaaatcaagtagcttggattttatcactgaatctattgcaaatgatttcttaaacatgctggccatggagagtggttcatttggctcaagttgcgatggtgatcccaagtctcccagagagaagcttttaagagagtttgaagaagaggctctagcttcaggtaacttcacctttgatttcattgcaaatgaagaggaattggggacaggtacagttgtagatagttatggggattgtactgtggactcagatttgtctttatttattcaagctgctgaagaggagcatgcaagggagaatcagttattgatgcagagaaggaaggccaaaattcttgaagacctggaaactgactcattgatgcaactttggggtttaaatgagaaggattttgaaaattctcagggaacttattctggtggttttggaagtccaattgaactacccaatgaagaatcctccgtattaccttcaattggacaggggcttggttcatttgttcaaactatgggtggaggctttctgaggtccatgagtccttccctgttcaggaatgctaaaaattgtgggaacctcatcacccaggcttctaatccagttgttttacctgcaaaaatgggtaatgacatactggagatactgcagcatgtggcttctgatggagttgaagaactgtgtcatcatatctataaattaatgcctttgcaggatatcactgggaaattcattgaacatattgtgcagaaagcgacaacagatgaaggggctcctgtaaggttagcaactgtgtttgtggcattacttgtgaaagactctttcatctaaagtatttgatttctttttttctgatttcctttcttaattctgtttcaggcaggggtcatggcagcatgatttgtttgaagaatttccatgtggttatttaacagatgaaggcacgagcttggacactgtgtctcctgaagctgtaggtcccatgactgttaataagattgaagcccgtttgatcgatgggttaagaatacagtctggcatgttgaatgaggaggcaccatcatatatccgtccccaacatgcaaagacacctgctgttggaggcagaagaaccaactggaaggggtttcccacatcagagagaattgctaaattgcaactagaagattgtggagaaagtggcaatgataatgatggattaatggggctgtcaataacatttgatcagtggttgagattggattctggcaccattgaaggagatcagaattcagagcaaattttgaagatccttgaagtacaccattctaaaatcagagagttagatgaattgaaaaatgccacggactggttaaaatcatatggcagaaagctcggacattatggactcttagggaaccatctaacagtggcttttatgatccagctcagagatcctctacgaaattatgagcctgttggtgttccaatgctggttctgactcaggtagaaagagtttgcattagtgcaacaccacaagactacagcgattttcttgatgaaaaggaaaagggaatggacaatgaggccctgctaaatgagacaagtagcaagagtatggaatatacgaatactgacaacgaagccactcaatttcgttttaaaatcaaagagattcatctctcaggagtcctcagcaaagccggaagcaagcaacgttggggtactgcaactcagcaacagtctgggatccggtggttacttgccagcggcatggccagtgctgccaaacattcatcttcaacatcaaaggcaattgtattatcatcccctctgtttaccaagaagttgctgaatgaggatgccctgtggagcatttcttgtgttaatagtatcatggggcctaatagtaaagagctgccagctgaaaatgtacacattcggaaccctgatatcatcttcccaagttgaactgtcagatcatttcttacatgcaatatagtcatttttttcttttttctttttttaaatttctccatgtagatagttgttatagtatgttaagttatacgagtcagcttgcccaaatttttaaattatatgttaaaatttctggtcttcttggaagcgacccttttttctttcaattacccttcctgatgtatactcaaatctaaagactaaacacagtttggctattttgtttggtttgatatgcaactgctaatgctgcttgcaattaataaaatgtgactgattacgtctaa

SEQ ID NO: 11 - DNA, 1564 bp, glycine max
caaaatgacagacaaaataccaaaaccaccaccaccagatacacctgacagtgatgatttcaccaaccagtggccttttgagctctctgagtacttgaaattcgatgataatcagtggatgcatgatggtcttgagtcatttgcttctgagaatgtctcaaaccaggttcatcaagtcagtaatgcaggtgagtttggaggaggaagcagccactttgaagggtcttctagcagtaagcatatacattagtagttcctcatgagatgaaagtgtgttttgttttgtagcttgagatgtttatatatccattttcaattactagttcaattgagtactatagtaattgatatgaacgttttcaactatgaatgaatgagtttaataatcatatatactgacacacaatgtaaattttttttatacggtcatccaattatatcaaattaattgttcctcatgagatgatgaaagtgtgttttattttgtagcttttgagatgtttatatggtcattttcaaatgacaaagtgcatgtttgggttaaagttaacaaacttaattttgagataatttttagtctgtaaactaagtttgcaaaagaaatcttgatcttgcttttatacaaacttggtttcaagtaaaatataagtgtgcaaatatacttttaaaaatgatcaaacatttgataaaattgagtttactttcaaaactgaatttggaacactccaaacaagaatccaaacatgctataatacaattgattactaaaataactgatatgaatattttcaactatttgtatattctcttttgcttatagttattggttgtgtgtaatattagaagatactagcagtggacgtgagaatagggaagttagagagagagttgcattcaaaataatgtcagagattgaagttcttgatgatggctacagatggaggaagtacggaaagaaaatggtgaaaaacagtcccaatccaaggtatgtgtgcctaatacatatttatataataaaatatggtggccttaattaaacatttttcgacaagagttcaaataaacgattaattagaaaagagagtaaagaaacacaaaacaaataattaattgcatacttaattagaactacttaatcaatgaacatatataatgcaatatattactacttaaattaaaaccacgatacttaagaaagatgcatgctatatatacaggagttttctattttatttttctttttatgaacaggaattactataggtgttcagtggatggatgcaacgtgaaaaagagagtggagagagacaaggatgacccaaggtatgtaataacaacctacgaaggcaaccacactcatccaagttcttcctaggtcgaatatgaagagaagttttggcagtgtatatattatgttttgttggccacatctattagtactttcttattaaatactgcaagtgcttttgccatgttttttttaagccatcatcaatgataataactaataataactgctcttgtagaatagatctttaaggaaatataggaaattaaagtatgc

SEQ ID NO: 12 - DNA, 952 bp, Glycine max
cacagcagacaattgctttctccagaaacttccatttccaaaacgcgcactttaagtagttatcttcagaaacttccatttctttctcgaattatctatgtatcccgtataaattgccacccacccaccctttttcgactacacagaaacattcgcaaaaacaaaatcccagtatcaaaattcttctctttttttcatatttcgcaaagacatggatttcagagtgatgggtttggagtctccactgttccacacgctgcaacacatgatggacatgtcagaggacggtgcaggagacaacaagacacacaatgctccaacatggtcatacgttcgagacgcgaaagcaatggctgcaacacctgcggatgtgaaggagtatccgaattcttacgtgttcgagatcgacatgccgggtttgaaatctggggacataaaggttcaagttgaagacgacaacctgcttctgatatgtggggaacgaaagagggacgaagagaaagaaggggcgaagtatttgagaatggagagaagggttgggaagttaatgcgcaagtttgtgctgcctgagaatgccaacactgatgcaatctctgctgtgtgccaagatggtgtgcttagtgtaaccgtgcagaaattgcctccacctgagcctaagaaacctaggactattcaggttaaggttgcttgaacaacttgtggaacgaagtttaataaatgattgttttccctttctggcagaagcagggcaagaagtaatgttgtttgaaggataagatggatcttatcttttagttgtttcaaggtttttctgttcttattgcaatggttctatggtgttttcagtttgtgatgttgtaatcagatgatgctattgctatatcagttgttcagcatttcttgatcttagttaattctctgttacgttctctgctttccaatttctatccca

SEQ ID NO: 13 - DNA, 769 bp, glycine max
tttagtctagcaagaaacattcccaaaacacagtaacaaaagtctttccttttcagcacttttttctttcatattacagaagccatggatttcagggtgatgagtttggagtctccactgtttcacacgctgcaacacatgatggacatgccagacgactctgcaggggacaacaagacatacaatgctccgacacggtcatatgtgcgagacgccaaggcaatggctgcaacacctgcggatgtgaaggagtattcgaattcttacatgttcgagatagacatgcccggtttgaaatctggggacataaaggttcaggttgaagacgacaacgtgcttctgattagtggggagagaaagaggaacgaagaaaaagaaggagcaaagtatctcagaatcgagagaagggttgggaagttaatgcgcaagtttgtgctacctgagaatgctaacacggacgctgtttcagcagtgtgccaagatgggggtgcttagtgttaccgtgcagaaattgcctcctcctgaacctaagaagcctaagactattgaggttaatgttgcttgaaccttaagaactaaattaaataaacgagcgtttcgcttttagacaaattcttgtgtgaagggtaaggggttgtctctgtaagaagcagagtttttacttttcagttgtttcaagattttttttttggtttgtttgtaatttcttattgaagtgatttgtggctttgtagtgttatgataatgaaataatatcagccaatgatct

SEQ ID NO: 14 - DNA, 2026 bp, glycine max
tttccttctcccacttctttctttattaaccatctctcaaacactcacacccaaccccatcgatcctttccttctcatgtccttttagctacactaaccttaaatttcttgcacttgtaaacctttgcatattaaaagctgcaacaaacgaatcaaaaaagtgtccaagtccaagatgagttcgattccaacaaccacctacgaagattcttcagcagttaacatttcagaattccaacctcaatctcacgcaccgaaggaaattgagtggcacatgctcgacaagtcaaagttcttcttcctgggagcagccttgttctcctctctctccgccgccctctaccccgccgtggtcttgaagacacgtcagcaagtttcctccgccaaaatctcgtgccgcaacatgtcacgcgccatcattcgctacgagggtttccgaggtttctaccgaggtttcggcacttccttaatgggaacaatccccgcacgtgcgctttacatgtctgcgcttgaagtcacgaagagcaacgtcggcaccgccaccgctcacttggggttttccgatgcgtcggccgctgccatcgctaatgccgccggaggcgtagcttccgccatggcggcacaattggtgtggaccccagttgatgtcgtgagccaaagactcatggttcaagaaagcaacaaaagtaatttaaatctaatccacgatctcaataattctgagtgtttttttgctcgttttttcagtaaagtttatttttttattaatcccccctttttttatttgtaatattcaaatataaaattttacttaaaaaatcttatatcaatttcactcaattcccattttgatcttttttccgtgtttcctaatagaatttttttaattacatttttttgcataaaagtcaaatttaaaattttacttaaacatctaataagaaaataatttttttatagttttttaatgtgtttgtgtaaattatttttatttaaaataaataatttttttcaagaaataaatcttcttggttacttaattttttttaagtttaaataaattcacccaatttcagttttactcaaaataaaaaatattttgatatttataagtgtcagtataaaaaataaaaatattaattcgtacctttataattatattgcgacatgtgaaattatgttatttaagatttttttttatcttctcttataatttttataaatataattttttttaaatttttaacaattactgttaggacgttggtctgtaagatcctagaaaaggtaaaattagcagcaaaataaattatatgcaatttttaaaaaaatcgctgaaatttcaatttaagtattaatgacaaaagactttgtcgttagattttgtcactattagtttaatttttttctttatagtgtgttacaggaatggttttgatgcatttaggaaaattctgggtgttgaagggccaagggggttttacagagggtttggggtttccatagtgacttatgctccctctaacgcagtttggtgggcttcgtattcaatggtaaacaggcttatttggggcgtgtttggtggttgtggtaattcaaattttggacgcgattcgaaggtgatggtgggtgtgcaagggttgagtgcagttatggctagtggggtttcgactattgtgactatgccacttgacacgatcaagacgaggttgcaagtgttggatgcagaagagattaatggacgaaggaggccattgactcttgtgcaagcggttcataacttggtgaaggaagggggaatcttggcttgttacagaggcttgggaccaaggtgggcttctatgtccatgtctgcggcaaccatgattactacctatgagttcttgaaacgcgtgtctgctaagaatctggatagattcattgtgtagtaactttaccagtaaaggtttttaggctaaagatccgttctgatcataaattgatatttgaaaaaaatttaaatgtacttcttgaatgtaaataaatt

SEQ ID NO: 15 - DNA, 5469 bp, glycine max
  misc_feature (1967)..(3001): n is a, c, g, or t
atggacgcaggttacaatcctcgtaccgtcgaagaggtctttagggatttcaagggtcgcagagctgctttgattaaagccctcaccactggttcttatcattttccatttcaaattttctttttcttttttcctttactttcaaaaaaaaattgtctctttttttgtttggcctccctgatgtttgtttgtttggttcttttctttgttctctttcttgcatgtctaattgaattggtttctgtgtggttcctccagatgttgaagagttctaccagcagtgcgatcccggtaacgtttcgttccgtagaaaaaaaaagattgtatttttattatttgttttggtctatgtttatgctgtgtgtgtgtgtttttttttttctcctgtctgaatattgaattgaactgatttctgaagaagtgcagagtggaaattgtattttgtttgttttgaatttgaatttagttgagtgttgaggccgtgggattttcgaacttaatttttctaaagtgttactttcacggttttgtaaatgactactggaagtttatatatatatatatatgtatatatgtatgtgtgtgtatgtatgtattatgtttttgttcaatgaggtcattttgtggatttctgtttctttttattattagttgttttaatgtaccgtggactgaaactcagaaactcagttgcaatttttttgtaaatgtaatttatattttgtttgtttcaattttaagaggaatttgaatttggttgagtattgaggctgtgggatttccaaaatttaattttcggaagtgtggaaaaacgctgtatacctaatttgcttgctttctcaattttgtaaatggctattacttggaagtttttgattttcatatctaatttgttttcaatggggtcatttgtggatttccatttctgcttctttttagttgttatagctttaatgtagcatcgactgaagtagtcatatgctaccacaaatgggaacaaactcaagtgcaaaaattttcagctggtagctttcaattttagcttgcatcattatgtttaattgtacatagccctgtaaactttgtttgaaatacggtgaattgggtttttgttaaatgggttcattttgcggatttttattaacttgccatccttcctttcataagaattacatttggtgggtcacaatataatgctagattttatttgcattttagattctttcaggatcattgtcctaatccttaaccctcctgagaagtatgaatatgacatgatgtggaatgagtatatgcacctcttttgtgtctaatcatttgtttaatgttaatggacttaagattcaccgtcagtaaagtgattattgggttcaagggatgtgcagataaccatttggttggtctttaatggaatgcgatcaagccatattatgcagatcatataggtacatctgggaattggaagtggtggtaattgaaacaaaatgatgagaaatggaagggaaggaagtttatgagattgtttagatgtttaaaattgtccaaattgggggagtattaaaaggttaatacaaattagttgaagactttggatgtgtggattatgtgagaccatcttatgttcctcctctcttgtgaaaaaaaaaattcaaatccaaaatttttacccaacatctggtatgtgatatgtaagttttcatgtgcaaagtgtgagctaaaacttaatcagtagcttagtgtttcttgattttatactccagtactctttagccctactgcataaacgtgagaaaatctgaatagaaggagaatgtgtacgttacttgaaatttattgaaactcgtgatttcagaagatagatgggttagtgagggatttattttgactggtataaggtcatttattgatatatgaagagtagcccatagggacattttcactcatgatactggatttagtcttttgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaaaggactggctgtctttggttgctgtgcacagtgatgcttggttgcttgctgtggctttttattttggtgctaggtttggatttgacaaggctgacaggtacctattttcctttctattcaatatgtatatgggtttaactgattattattttttctcttaaatgatgcatgcatttgatcttgggcgatattgttaaatggggatgctatggtggaattgtgtggtggattttttacccaccgccataggcaatttgtgaagggaggcctgttggttgtgccacaggctgcaatggaggaattttgtccttcggccatattccaccatccaccattgttgataacaccaaccttaggtagcttaggcttgggccagctgtttggatcattgggacgctgttttcacattatttctaagttttaaactcaaggatggtttggacctattcttgaaacatagttcttttgctgttttgtttgcatgccagtaaacagattattagggtaaagaaagaaatggttagacacagaagaggtgcatgcataagcaataaagataagagtagtaggaagtatctgactgtgtagtactgaatgtccttaattgcctcatgtaatgttgtctaccttgacagttgcaactggttttcagtatggtgttctttggctcatttgaggaaaaattaactgagttgaaataataggattgtgttgcattgttaatgggattttggttggtgtctttctttattactagtgtggaacccatgttatgaaagatgtttgttaatataaaatataattcttagtacatagtgtttatgagttctaatggcattgacaaaagaatattgcccagcaaagctgagaagagtcatcatattcattctaaaatgaagaatagacgaacaatgggactgttcaatccagttccaatttcgttagagttgaaaaccaaacatgtagtttataatgcctagtagagctggatataacttaagaattgaggctttggtgagagataaaactgtggatatgtctattcaaatatcaaataatatattaaatgttaacaatgtaattgttttctgtttccctataatttatttccttctagtagaaaaacattttctcattacctagatactcttctaatcttcagtttgcattcttttgtgacttactattgatggctttacctctatatgttgtaacttgtaattaatttgagaagttgtcatctttcagtaatatctatttctcatcttcatgcaggaagcgactatttaatatgataaatgatcttccgacaatatttgaggttgttacagggatggccaagaaacaaggaaaggaaaagtcatcagtttcaaatcatagcagtaccaaatctaagtcaaactctaaggtaagcaagtctgaaaatcaagctgctctcactagctgtagtaagtagtatcatatagcttttagcaatgaagctaattcctaaattttgtgcatgttcagatgcggattattttttagaaatatttttattttgtttatttttttctgaacgttcacttgaaaagctagaaaggtaaagcaatttcccctccttaaaaataggctaataatcacattttgttttatacttgatttaatgatttaaactttaggtgtgttagcaggggttcttaattcagggaaagggggggtgaattatttaagtgattgtcctttactaagtagtgtggtatgttgttttttagatgctatttgaacttaggagtggaaaagtacttactttgtagggagccaaaaaaagttttttactatttgtaatgtgatctggtttcttctgattggtatttgactatatatttttgttaatctcttttgggttgacaaatgcagcgcggttctgaatcaaagtacacaaaagcaatgcaatccaaggacgaggacgatgaaggtgtgggtgtggaagaggaagacgaagatgaacatggagagactttgtgtggggcatgtggagagagttatgctgccgacgaattctggatttgctgtgacatctgtgagaaatggttccatggaaagtgcgtgaagattacccctgcaagggccgagcatatcaagcagtacaaatgtccatcttgcagcaacaagagagctcgaccttgacaacatcatggctaggatggttggtgactataatcttggcaattttatgactactaatttgtttagtaggtcaagtttattagtcttgctgtgaatttcaggtatattgcttcagatgttattacattaggtatatgagctgctgtcgattcatctaccattaggatatttgtttcataagtggacttgaatgttgttttgttatgtgtttctttaaatttaatgtagctgtgtaactgtgttgtgtccatagcttagtgaagtttatattatctcccgtggtgtaattctgttc

SEQ ID NO: 16 - DNA, 1484 bp, glycine max
aaacactactacctcttccttaattttcttcaatcacaccaaatttaactaccttaagcttttcacaatttccatagtttccttgtttccaattattgcatgccaatcatgtacaatttagagaagccacagagagatgataaacagtttctaactccctctttctcttccactctcctcgaccaaatttatcgttccatcgacgagggtgaaagaaaaaacggcgaaacaaagttctacagacacactactatgagcagtagcaagagacacaacagaagcgattcgaaatccatggacgctggagacagaaaaattgtcggcggcgcgaaaaacaacaacagaaaacatctgcaccgcgacgaggatgtcatgttcttcagctccacttcgatttcctccacagattcctccagccttggattctcctcctccgacaccgaatccatctcacgcgcctcgtgtttcgcgccacaggtggggcgcggaggaggaggaagcgcttcattccgttccgagaaacagggaatgcgcatcttcgacagtttctgtcgaacctcctcccatagatcgtcggaacacgcgcattcccaattctgcatcacaagccgtcgtcatctgtccgtgtcagaagatcataaacaaatcagacacggacacagacaggtgacggcttgtgacgaagaagaagaggccttaatgatcaaatccaagtcgagggccttaaagatttacaacaaccttaaaaaagtgaaacaacctatttcccctggcggcagggtcacgagtttcctcaactcgctcttcgccaacacgaagaaaaccaccaccactactacttctacaagttcccgcacgtgcggcgaagtgaatactccttcttcttcttcttcatgttattattcttccacgtgttcctctgcttcttcgttttcacgttcttgcttgagcaagaccatgtcttccgaaagggacaggttgcgcaatggggtgaagagaacggtgcgtttctacccagtgagtgtgatcgtgggtgaagatagcagaccctgtgggcacaaacgcttgtgcgaagagaaagaagcttctagagagttcttaagagagtacaataaccgacacaaccccaagattaagagcaatgataaattggtcttgaaggacttgtctttaaggactactaacgttgatgttgatgataacgatgacgacgatgatgcatcgagttatgcgagctcggatctcttcgagcttgatcatttggctgtgtttggaagtgataggtatagtgaggagcttccggtgtatgaaactactcatgttagtactaaccgcgccattgccaatggcctcatagtataataattctgttcaatatacgtacagaaccatttttgagttttttcccccactttttttaagtggttttgttaattatgtatgattatcaatattgggtaaattaataaaatgattttgatcaa

SEQ ID NO: 17 - DNA, 6025 bp, glycine max
atgagtttcaagaacagtcctaaaattcagcacataggagcctctccaaacgtgtctcgccaatcatccacttctagttttagggacaaacaacagaagaatgtggagccaccaagccctttacctgttggatgctgcagaggcttcagcattgccaagactattgggtcccctgcctccacatcacaacaaaatatgcaacagttacaataccaaagacttgaagtaaaggtatgtttcatgcatttcaagtagtgcacaactgcacatacatgtattagtgccagattggtttcatgttgagaaattgattcttgagtttaggattaattttggatacatttttacatatttgattttactttggacaattcagaattcattctgaattcaaaattgattatgagatatcacaactttaagtaactttgcattggatccaaaattttatattaaattttatttttaacttgattttataataaaaatatttaaatataaattacatcacttcaaaatcaattttatccatgttcatctaaatacacattacacttataacctaaatttaagttttgcttcaaatgaatatatcaatgattcttcctgattcccttaaaaaaatattcttccggattcaagagattaatctgaaaagacaaaattaatacatgtgctagaagtctaaatttacatgcattacttataggtaatacatatagtctaaggcgaacgcgtccaatacgtgcgttctctttaaaccaacctaagtctgcacatctctgtaataaaataaaaaaaatgtctgcacatctctacttggttgagcatataaatttaagaaagagaaaaaatatggcagaatgaaaactgtttttaaccaaaattgtaaaacaacaaaatgtaaatggcatttggtttggttgttttttacttttattttcactgaaaataaaaaatagtgatgaaaatgtgtttgactggatttctgttttcattttcagtgaaaatattttttcaaacgaaccaaaaaatagaaacaataaaaacttgttttccatgtttttagttgaaatcaggaatttcatattgggtaaaatgaaaacatgatgacaagaaatgtaattttaagcaaatctaaaaatacattttcttttgaaaaccagtatttttatttcttgaaaatagaaaataagaagttaaaccaaacatattttcagaattctaatcttttaaaaataaaaacaagaaataaaaacagaaaataaaaatgcaaactaaacacaccctaaatttttcatctttcagttccatttcattgtcaatcaccaaagcagactttataagcagaaatcaagaaggacgcacaagttgctgcagtatatatactaatttcttttgagaaaactgaaattattaaactggtcgattaaagaagtctagccaaacttaaatttctataatgagaatcataacacttttatgtagttttaataatggctttgaaggaattaataattaagattgattagaaaattttcgtttttgttaggcatctctttgaaactatgtagcataactgatgatatattctgtttagaaaaacttatttttaagagaatttatctctaacgcaacatttttattgaattcattcatatctctcatattccaatatatttaattttcattttcaatattatataaaatgcaacatatgccaagaatcaagaactatatttactttgacttaaacaaaaataactaaatttacttttgtaatattggagtattaaaatcggccgcggggtacacggagtggagaatagtagggccggagaaggtgttggatgaagcctgcgaaggctattgggctgaaagaaactaggctaattaatgcttaatttttggagtgctaggtgcaccctgtactaatcacaaaaagacagaaatactccttacggatcaagttgatctgtaagttaatttttaagacttacagatcaagttgatccgtaagtcttttacggatcaagttgattcgtaagttaatttttaagacttacggatcaagttgatctgtaaggggagaaattagcagagacagttttgtcattttcaaaaaatactgggtgcacctaacaatattttaatttttgtggcattgcatcatgtaattatttaaagcaccccattatttgatttgatgcaccaaagtgtattacagattgaataatttgtaatacaacataatgcatttgagatgcaccaaagtaaataatggggtgcgttaaataattcccattgcatttggatccaaaaatggaccaggatttgcctagtggtttaatacaataataacttaaatactaatgacaataataaaaatatcatcagataattctcttaatgaaaatactaattagaattggaattctatctctgcaattagtataataaaagtttgcatttaaaaattatacagttttctaagctaaaacataaattccgattgaagaagaacaaaataacacccaagatattgtatttaagttttgaccattaaaagaaaaaagcgtctgaaatatgtgttcacgcctgttcataggtggcggatttggaaaagaaagtgcaaaaacaatcagaactacgagtaatgtacaagaaaagaatggaaaggactcaagattacttgaggaactgccttcagatagcacaagaaaatggaatattagatgtcattgtgaactgcaaaggtgaatttcaacaatctccagtctctatccacaccataagctccattaccagccctcaaattcaaattcccactccaccacaccatggccaccacaccaatcttgcgtccatcatagatcaagcaaaaatcaatggatggtatattgatccaaatgaggttagtaagtagaattccactcacatgcaaatcattcacttacaagttacaatgcgttgtcaacatttttttttcaattaagtattcaatatgtttccatctccttgctttgattctgtttttttttttgttttttccttttttcataaatcatcatgtttgctttttatttcttaagttttgattttacataatgtttgccccttctgaatgttagaacaaatataagctattaaattagtgctgacaaaatatgttagatcacctgagttggctcaaacttggtcgataaaatgtaatacttagagttttaaattttttgtctcaattaaacatgtttttttatattgccaaaatgcccaaaaaaattcaatttttaccaagcttgaatttttgctgttgtttcatgcatctctaagttacttactgccccttttttttaacttctggaatgtaaaattacattccaaaatgatgaccaatcccggtaaaaaaaaaactccaaaaaaggtacagaaggcaaaaagggtacagaaagcaagaactagacaattttttttttagcctactataatccacaatcagtattgggctgacctccattggattggaaactctgtaacactttattttcaacacattctattttatttggtgaaatttatgtgagtacttattaaatatgtgagttacattctccatttgttatatcccatgcccaaaccagaggaatctacattaattttaatttttttttaactaataaaggtggttttgaaagaaagtattaaaaactgtattaccaatactcttctcaaaacaattagatatttttttataaaaaaagagcaaaaaatgacatttcacaaacaaataaataataaactaaatgaagaattataaaaattgtgagaattgaattataatttctgaaaaattaggcaataaactagaatttgaaatatttaaaatgaataaaaacatattaaagtgttgaattaataagaaaataactgctatgttatgttatgttatgtctgaacttctccatgcatagattcaactggaagagaaaatcgggcagggcagcaccgcagaaattcacagaggaaaatggcgtggatttgaagtggcagtgaagtgcatatccgaagaatttttccgcactaaccaaaacggtgtcgcatacttctcccaggagctcgaaacgttgtcgcggcagcgccacaggttcgtgcttcagctcatgggcgcgtgcatcgaccctccccgacgcgcgtgggtggtcacagaacatctcagcaccacgttgaaggagtggctgcacgggcccgggaccaggctcagagagcgaatggtgccacttccactcttcaaagacagagtcattagggcccttgaaatagcccaagccatgcagtatctgcacgagcaaaagcccaagctcgttcaccgtgacttgaagcctagcaacatttttcttgatgatgccatgcatgttagagtcgcggattttggtcatgcgcggttcttgggtgatgaggaaatggctctcactggagaaacaggtaaacactgaatgtcatttcttttgctgcaaattaaaaataaataaaacgttatgaaaaataatattatattttactgtgatattttcgtgtttaagtctcattttaaaagttcagtatcttgtactttaactattttatcctccatctcatattgtagttttgaaaacccatttagaatgtacttagaaatataataggcaaaattgtaaaattagtctcccactttatcttcaatttcagatttggtttccaagttttttaattcacgaatttagtctccctattttgtaaaatcgtacaatgttggtcccctgggtcacaattggatgttgacgattagcaagttatgttgactgtcacatgtcatgttctgattggatgtcaattccatgaaaaatgaaatgcattgttgttttcattgaccaatcagaacatgacacgtggtcgtcatcatccaataagatcatgacacgtggccgtcaacattcctttgtaacagtcaacgtccaattgtggcctgggagaccaacattgcatgattttactaaataaggggatcaaattcgtgaattaaaaagttgggggaccaaatctgaaattagagataaagtgggggaccaattttataattttgccaatataatacaatgagatgtaagataaaataataaaaatgcaagaaacaaatatctaaaaactggcttgaacttatttcaagttttttctagctcaatccattgtaataactttcaatcaacatcggactcacttgtattaggttgtattccatcttagtatctctaatgctttttcaagtgtttgtgtttggtcataacctcattgtcattaatacatgacaatttgtgataataaagaaattagccataaagaccattttcacctactagctaggatgtttgaaaatcaagttgtacatttttaaattttagatgagtattaaatgacataaagttgaattgtgtaaactatattctttctccttttctcaaaacgtggcatgtaatgatattgattatgtttgtattatttttaggaacatacgtgtacatggcaccagaggtgatccgatgtgaaccttatgatgaaaagtgtgatgtgtacagttttggggttatgttgaacgaacttttaactggacattacccatatgttgagacagaatatggtcccaccaaggtatatatatataatcatcacactgatcttgagttgccaaattaattttgttaattttttatgtaaatgtttaattgaattgtttaatgctaatctcaattaatctgaaatgtcacttgatgttgattatcatgaaattaaatgtttgatgttataatattagattgccattgaagttgtggagggtaacctgcgacctaagcttccgtgtgatgatgttggccaaccgagagagcttattgaccttatatgcctttgctgggacaaaaatccatctactcgaccatcttttgctacaatcaccctttgcctcaaaagttatgccaagaaaaatctcctttaa

SEQ ID NO: 18 - DNA, 5565 bp, glycine max
tccataaccacattgcacttttcatccatttctgcgaaatcactgttttgttgtctatatttttttttttccctgacatgggtgaagctccagaggtgttctacattaatgggttctcaaagtttgaacatgaaattgaaacccaacaaaaccatcatgtgaatgatcagaaccatgattcttcttggtacgaggaaatcatcgatgaggatctaaaatggtcttttaaactcaacaggttggtcatcattgcttctgcaattcactactccaaatcaatgtatattggttcaaacaaaaaaatattatcattttcattttgctctgcctctgccttgcttgatagatataatttttatttttgtttcattactttaatgcatttcttttatttttattttttcaatttcaacagtgtgctccataaagccattagcgagtaccaggacatagctctcttagatacaaagcgttttggaaaggtttgcaatctctgcttcttcttctcataaggacagaacaaaaagaaataaataggatattctgaattgctttcttctattctagttgtttgtagtattgagaaaaaagagggagggaaaagttttaaataatattgagaaatgttatatattaagacattttgtagcacgtattctgttattagttagagtgaaatttaatatacacagatgaagtgaattccaggtcttatttgatgtttaaacttcaacaattttattaagttttaactactataaaaaaaaagtgtttgttactaacttgatgataattgttgatgttgcatacaggccttggtgcttgatggaaagatgcagagtgctgaaacggacgaatttatttatcatgaatgcttgattcatccacctcttttatgccatcctaagtaagagcatgatggttgaatgatgtttgagtttattaatattcatttctggagggcttacaatgttccttctttttttttaaagcaaagcttacaatgttacttggaatttaaaaacttcatcaattcttaattcttaaatatttaaaatgagttatacaattgtttgacttcttgaaaaccattaagattgcttatatatacaacactatcatgccaattactccaatattaaaattgcataataagtattttttaaaagtttaaaaaacccattctgttgaaaatgctataagttccaaatgataaatatgacataatgttttgagtccagcctttgcgaaacctgttttttaggtgaaatgataatactatattaattatggtttgtaaataaataatttatttattttctaaaagtattaaatattgcattaaatattatacccaagttcacacatagtttgatctctctttattttatatacagtaggtatgcgtaaacgggcgtgcgccagtgcttttataatgatgggattttggtaaaggataattttcgtggactacagaaaaggagattttttgtttttcccatgatcattatatttactaccctcctgattaataaaaaaattggtcaaattatttaattttttatagttaaaaaatgttgtaaatatgaaattgaagcaattcaaggcgatgtttgaataaatatttttggaagaagaaaataatacaaaaatgaaatgaacttctcacataaattaaaattaacttatataacttatatataagttaatttgtagaagttttcttattttattttctccaaaaaaattattttaacctatatatgagttaatttcaatttattaaaaaactttattttttcctataaatatttatagaaagttttatctaaagagcacctaaatgaatgaacatatagttttcttaatggtacatgtactcaagccgagtaacgaatatatatatatatatatatatatatatatattacatgatacatgattatgcttcttggtagactctattgggcttatgaacccttcactaatatgaagagacatatgttaatgattatggcagtcccaagactgtgtttataatggggggaggtgaaggttctgctgctagggaagcccttaagcacaggtcaatggtcagagtggtcatgtgcgatatagacaaggttggttactaccataccatattattttgcttcttagccgttcatcaatttcaaacgttaattatatccatattaatatgggcagagatcaatgtgtatttttttaatatataaaagatttaattatgaatttagtatctataatttttaaacttatctctgtagattttttagtttttgaagtttaaattttaattatcaaaagttcttggttgttagattttttttaacttcattaattaaaaaattttaacgataaattttttttgagaattaaaatataaaccctaaagactaaaaaattcacttatgaattatataaactaaaaggaataaatttaaaaactataaagactaaatgataattaaacttatataaaatatatttgtcttacctatactttttttttgtaaatcttatttttagtcttcatgtaattagtcattaaaattctataaaaaaacatatttttagtctcttctaatgtcatatgagagaattatgtgagaattgactaaacacattttttttatatataaagttcaaggattacaatataaaaatttagagattaaaaataaaattcaacgaaaatacagtaactaaacatattttatcttattttatatatatatatatatatatatatatatatatatatatgcactcaactataaaatattattgaattttgttggttttcacaaaaaaaaaatgtcggaagtgtataaattattatagtaacaagtgaatgtttgataagttgagatgtcaacgttacaacaagaatttacactatatcctattcttaagattgatgaagacactcaaataaagtcaatggaatttcaacttctaattttcgcaggaggtggtagatttctgccgaaaatacttgattgcaaataaggaggcgttttctcataagaagctcgaccttgtcattaatgatgccaagtaagtgtaacttgcttttcttagtaatgattttgacattttacctttcatttatatattattttttaccacaaaactatagtgttggatagaatcaattttgttagccttcaaaattttaagatgaaatatatatctatttatcactaaattattaaaggttttattttaagcttttaagattaatatctatcttcctcgacaaggttaaataaactatctctatagtccaatgatatgtctcattttttaagggctgaattggagaaaagaaaggagaagtttgatatcatcgttggagatttggcagacccacttgaagatgggccttgctatcaactctacaccaaatccttctacgagaagatcctgaagcccaagctcaatgataatggcatttttgtgacccaggtaggtagattatgaatatgagtaacaccctttcaaggactctgttgtttcctgcactttattttttattttttactttcaaattgatcaatctagaatataaaattaaattccgaaatgattctaaattttggtttctgaaatgatcaatctaaaagataaaaaatatttccaaaaaagtgtaggaaataactcataggtgcaggaagaaaccaccttttttaagtcatgattagagagttttttctactctcctactcaataaagtttaataaaaacatttcattttgtgcttaggctggaccagcaggtatcttcacacacaaggaggtcttcacttctatatataacacagtcaaacaggttttcaaatgtaagactatctaattcaagtcttttattatgggaaagaatttcagatatcattaacagaattaacagagcacaaggatattgtctttaagctgaaactaaagttttatttttttggtgcagatgtgattgcatacacgactcatgtaccatcatttgctgacacatggggttgggttatggtatgcgaaaaaaaagccttgtttctttttgtacttataattaaatactccactaaactcttgttcaagttattgtcattacatgtgtcaattttttttttccgaattccttaaaattttaatttactttgtgacttaaaaaatttcattcactgttattcttaatgttaattaatgttttttaacatgaatgaaacttttaaggccaacaattaaaaataaaatttaaataaagttagataactaaaatatctaatgtattaaaataattatatatttttttaactttccgatttataaatagactctaactaatatgaaagattgagaagtaaaaaaaaatcaactaaaaattatttctatcaaagaaattgaataatattcaaacaatttaattttaattttaatatattaatcacttttatatataatatgagagactaaaatgaagtactcttaaatattattcacaagaagttttatgctttcaattcaggcttcggaccaaccactatcgatttgcgctgatgaaatggacagaagaatcgaagcaagaatagatggggaacttctctatctaaatggtgcttggttccattcatctacaactatgaacaaaactgtttaccaatcgtaagtacttgcaactcaaattcaaacccaaaaatcttaaataaaatttaaggccctgataaaccaagttaacattctcttgtcagaacattattcaatgaacaatttagatgctgatattaacatgttcatcactttattggattttgacttccttttttcttaatcattgttgcaggctgcaaaatgaaactcacgtgtacacagaagaaaatgctagatttattcctggacatggcatggcttttcatctctaagatttcaagccattgatggagagaaaccgttccaacctcaagttcattaaattgttaggcttattacatttaagaaattattcgctttgactctcatgagtcttcacaattttgtttttcataaatgccttaataggttaaggaaataaaatatttataaactatctctagtctctcagactcattaataatgcgagacttttcttttttatgtattggaagagtagccctccttctagtgtcaaaattccatcaatctgaaccgagagctattgttccaatacattaaaaatcctacatttcttagcaaacaattccaatgatagtttataaatatttttctccctcaagatatgaagacatcttttaagatcaaaggacaatacttcaaatgtggcgagtttaacaatataatagacttgaaatcggaaacaaaaactcttgcattctgaccttcacctcaaattattggatcgtacagtccaaatttaacttcaaagttaatcatgagagagcacattcaattcttgtaatctatgcatattatcatgcatgttttcatttgtaaaatggtgttagagtatgtggggatttgctgtatagaatacaaagatattggaaggactagt

SEQ ID NO: 19 - DNA, 121 bp, Glycine max
  misc_feature (61)..(61): n is c or t
gaagttagatctagttggcctctcattggtgttatgcccgaagaattgctgcaagactcanagaaggtatctggggtacgctaaaaaggaaagtgatacatcgcatgtgcctctacaatga

SEQ ID NO: 20 - DNA, 121 bp, Glycine max
  misc_feature (61)..(61): n is c or t
attgttctcaactcagacatcggcaatggagttggacgagccacctatggccaacctctgngcttcaaaaactcttctaacggacatgtcacagatttttctactcgcttttcgttcacca

SEQ ID NO: 21 - DNA, 121 bp, Glycine max
  misc_feature (61)..(61): n is a or c
aatcctcccaagaattcatacaatgtgtaatgaatatcaaactaaaagcctagaaatgatntactctctcacagaacaactgcttcaattcgtccactgatgactcttcatttgcactcta

SEQ ID NO: 22 - DNA, 121 bp, Glycine max
  misc_feature (61)..(61): n is a or c
aagagttctaaccaacatatccacgtcgttcaccttcaccattgaaagagctgcaacaganaacataggtgacggtttcaccttttacataggctaccagattcctccaaatgcaactaat

SEQ ID NO: 23 - DNA, 121 bp, Glycine max
  misc_feature (61)..(61): n is a or g
tgaatataataatgtgtaaaatgcattgaaatgagacaaaatgaaacgaagtgtaatggangtaactgataaagcaaaagagaaagaaaaaaaatatatatttttcattatattgttatga

SEQ ID NO: 24 - DNA, 121 bp, Glycine max
  misc_feature (61)..(61): n is c or t
aaaaataatattaaaaaattcgtgttaatatcaattttacaaaaaatcaatgttaaaaaantcgtgttatttatgaaattgtcatcacatttttattaatatctattttatgaaattaaaa

SEQ ID NO: 25 - DNA, 121 bp, Glycine max
  misc_feature (61)..(61): n is c or t
gcggggattgttcccattaaggaagtgccgaaacctcggtagaaacctcggaaaccctcgnagcgaatgatggcgcgtgacatgttgcggcacgagattttggcggaggaaacttgctgac

SEQ ID NO: 26 - DNA, 121 bp, Glycine max
  misc_feature (61)..(61): n is c or t
catggttaacgtgtgatcgatgaactcttctagaatgtattcgaaagatgggaattgaaantataattttaattaagcctttttagaagttaatataaaatgtatattttaatatttgagt

SEQ ID NO: 27 - DNA, 121 bp, Glycine max
  misc_feature (61)..(61): n is c or t
ccttccccatgaaacagagccaatgggtgagaacgataacaaaaccaaaaaaaacctcctntccctcctaacagagccaatccaatggatccaaagtctctcctcacagctcaacccaacc

SEQ ID NO: 28 - DNA, 121 bp, Glycine max
  misc_feature (61)..(61): n is a or g
gcctgatttatattttgtcagaaggatatccagaagttacttgctgcctgagtgtaattcngaacacaaacgagagctgtatgtaagagcacgaaccgagtgatgtgtgcacaataagtta

SEQ ID NO: 29 - DNA, 121 bp, Glycine max
  misc_feature (61)..(61): n is c or t
ttatggaaaagaaggtaaatgagagggggccacttgtcattaaactctactaccccccccncccccccaacttgggagttgataaaaggtaaaattgtaaatgacgattccaaacatagcc

SEQ ID NO: 30 - DNA, 121 bp, Glycine max
  misc_feature (61)..(61): n is c or t
aacttattttatttataacttttgcgaggaaactccaatttaaatgaaatttaaggataancgtatgttttaacacccaaagaactcatttttggcataaaaaactcaaggaaaacatctc

SEQ ID NO: 31 - DNA, 121 bp, Glycine max
  misc_feature (61)..(61): n is a or g
cgtaactatcacacttatttcacaatagggcctaatcactgccaccaatcctcccagtgtntctctatccatcatccacgtccttaatgttggatcaagtggtctcggaataattaagaaa

SEQ ID NO: 32 - DNA, 121 bp, Glycine max
  misc_feature (61)..(61): n is a or g
ccttctccttacaccaaatacctttcttaaagatagctagcctagaacgtcttacgtcctngtgttgaacctttcctggtttccccgaatcttgatatctataagaagcattagatgcact

SEQ ID NO: 33 - DNA, 121 bp, Glycine max
  misc_feature (61)..(61): n is c or t
tcacattctgttgaaacactaccaagcaagtcagagacccgacatggagtgcgtgtaacgntgggggattatttatttgagaatgttaccatttttagaaaagattttagttttatagtaa